SEQUENCE LISTING (150 sequences)

SEQ ID NO: 1 - DNA, 618 bp, Homo sapiens
  CDS (1)...(618)
  misc_feature (0)...(0): IL-28A
atgactggggactgcacgccagtgctggtgctgatggccgcagtgctgaccgtgactggagcagttcctgtcgccaggctccacggggctctcccggatgcaaggggctgccacatagcccagttcaagtccctgtctccacaggagctgcaggcctttaagagggccaaagatgccttagaagagtcgcttctgctgaaggactgcaggtgccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtgtgagtcgtcagggcctgggcacccagatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctga

SEQ ID NO: 2 - protein, 205 aa, Homo sapiens
MTGDCTPVLVLMAAVLTVTGAVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACVSRQGLGTQIQPQPTAGPRTRGRLHHWLYRLQEAPKKESPGCLEASVTFNLFRLLTRDLNCVASGDLCV

SEQ ID NO: 3 - DNA, 603 bp, Homo sapiens
  CDS (1)...(603)
  misc_feature (0)...(0): IL-29
atggctgcagcttggaccgtggtgctggtgactttggtgctaggcttggccgtggcaggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggacctgtgtctgagaacgtcaacccaccctgagtccacctga

SEQ ID NO: 4 - protein, 200 aa, Homo sapiens
MAAAWTVVLVTLVLGLAVAGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 5 - DNA, 615 bp, Homo sapiens
  CDS (1)...(615)
  misc_feature (0)...(0): IL-28B
atgaccggggactgcatgccagtgctggtgctgatggccgcagtgctgaccgtgactggagcagttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtgtgagtcgtcagggcccgggcacccagatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtc

SEQ ID NO: 6 - protein, 205 aa, Homo sapiens
MTGDCMPVLVLMAAVLTVTGAVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACVSRQGPGTQIQPQPTAGPRTRGRLHHWLHRLQEAPKKESPGCLEASVTFNLFRLLTRDLNCVASGDLCV

SEQ ID NO: 7 - DNA, 633 bp, Mus musculus
  CDS (22)...(630)
tcacagaccccggagagcaacatgaagccagaaacagctgggggccacatgctcctcctgctgttgcctctgctgctggccgcagtgctgacaagaacccaagctgaccctgtccccagggccaccaggctcccagtggaagcaaaggattgccacattgctcagttcaagtctctgtccccaaaagagctgcaggccttcaaaaaggccaaggatgccatcgagaagaggctgcttgagaaggacctgaggtgcagttcccacctcttccccagggcctgggacctgaagcagctgcaggtccaagagcgccccaaggccttgcaggctgaggtggccctgaccctgaaggtctgggagaacatgactgactcagccctggccaccatcctgggccagcctcttcatacactgagccacattcactcccagctgcagacctgtacacagcttcaggccacagcagagcccaggtccccgagccgccgcctctcccgctggctgcacaggctccaggaggcccagagcaaggagacccctggctgcctggaggcctctgtcacctccaacctgtttcgcctgctcacccgggacctcaagtgtgtggccaatggagaccagtgtgtctgacct

SEQ ID NO: 8 - protein, 202 aa, Mus musculus
MKPETAGGHMLLLLLPLLLAAVLTRTQADPVPRATRLPVEAKDCHIAQFKSLSPKELQAFKKAKDAIEKRLLEKDLRCSSHLFPRAWDLKQLQVQERPKALQAEVALTLKVWENMTDSALATILGQPLHTLSHIHSQLQTCTQLQATAEPRSPSRRLSRWLHRLQEAQSKETPGCLEASVTSNLFRLLTRDLKCVANGDQCV

SEQ ID NO: 9 - DNA, 632 bp, Mus musculus
  CDS (22)...(630)
tcacagaccccggagagcaacatgaagccagaaacagctgggggccacatgctcctcctgctgttgcctctgctgctggccgcagtgctgacaagaacccaagctgaccctgtccccagggccaccaggctcccagtggaagcaaaggattgccacattgctcagttcaagtctctgtccccaaaagagctgcaggccttcaaaaaggccaagggtgccatcgagaagaggctgcttgagaaggacatgaggtgcagttcccacctcatctccagggcctgggacctgaagcagctgcaggtccaagagcgccccaaggccttgcaggctgaggtggccctgaccctgaaggtctgggagaacataaatgactcagccctgaccaccatcctgggccagcctcttcatacactgagccacattcactcccagctgcagacctgtacacagcttcaggccacagcagagcccaagcccccgagtcgccgcctctcccgctggctgcacaggctccaggaggcccagagcaaggagactcctggctgcctggaggactctgtcacctccaacctgtttcaactgctcctccgggacctcaagtgtgtggccagtggagaccagtgtgtctgacc

SEQ ID NO: 10 - protein, 202 aa, Mus musculus
MKPETAGGHMLLLLLPLLLAAVLTRTQADPVPRATRLPVEAKDCHIAQFKSLSPKELQAFKKAKGAIEKRLLEKDMRCSSHLISRAWDLKQLQVQERPKALQAEVALTLKVWENINDSALTTILGQPLHTLSHIHSQLQTCTQLQATAEPKPPSRRLSRWLHRLQEAQSKETPGCLEDSVTSNLFQLLLRDLKCVASGDQCV

SEQ ID NO: 11 - DNA, 1563 bp, Homo sapiens
  CDS (1)...(1563)
  misc_feature (0)...(0): IL-28RA
atggcggggcccgagcgctggggccccctgctcctgtgcctgctgcaggccgctccagggaggccccgtctggcccctccccagaatgtgacgctgctctcccagaacttcagcgtgtacctgacatggctcccagggcttggcaacccccaggatgtgacctattttgtggcctatcagagctctcccacccgtagacggtggcgcgaagtggaagagtgtgcgggaaccaaggagctgctatgttctatgatgtgcctgaagaaacaggacctgtacaacaagttcaagggacgcgtgcggacggtttctcccagctccaagtccccctgggtggagtccgaatacctggattacctttttgaagtggagccggccccacctgtcctggtgctcacccagacggaggagatcctgagtgccaatgccacgtaccagctgcccccctgcatgcccccactggatctgaagtatgaggtggcattctggaaggagggggccggaaacaagaccctatttccagtcactccccatggccagccagtccagatcactctccagccagctgccagcgaacaccactgcctcagtgccagaaccatctacacgttcagtgtcccgaaatacagcaagttctctaagcccacctgcttcttgctggaggtcccagaagccaactgggctttcctggtgctgccatcgcttctgatactgctgttagtaattgccgcagggggtgtgatctggaagaccctcatggggaacccctggtttcagcgggcaaagatgccacgggccctggacttttctggacacacacaccctgtggcaacctttcagcccagcagaccagagtccgtgaatgacttgttcctctgtccccaaaaggaactgaccagaggggtcaggccgacgcctcgagtcagggccccagccacccaacagacaagatggaagaaggaccttgcagaggacgaagaggaggaggatgaggaggacacagaagatggcgtcagcttccagccctacattgaaccaccttctttcctggggcaagagcaccaggctccagggcactcggaggctggtggggtggactcagggaggcccagggctcctctggtcccaagcgaaggctcctctgcttgggattcttcagacagaagctgggccagcactgtggactcctcctgggacagggctgggtcctctggctatttggctgagaaggggccaggccaagggccgggtggggatgggcaccaagaatctctcccaccacctgaattctccaaggactcgggtttcctggaagagctcccagaagataacctctcctcctgggccacctggggcaccttaccaccggagccgaatctggtccctgggggacccccagtttctcttcagacactgaccttctgctgggaaagcagccctgaggaggaagaggaggcgagggaatcagaaattgaggacagcgatgcgggcagctggggggctgagagcacccagaggaccgaggacaggggccggacattggggcattacatggccaggtga

SEQ ID NO: 12 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 13 - DNA, 1476 bp, Homo sapiens
  CDS (1)...(1476)
  misc_feature (0)...(0): IL-28RA splice variant
atggcggggcccgagcgctggggccccctgctcctgtgcctgctgcaggccgctccagggaggccccgtctggcccctccccagaatgtgacgctgctctcccagaacttcagcgtgtacctgacatggctcccagggcttggcaacccccaggatgtgacctattttgtggcctatcagagctctcccacccgtagacggtggcgcgaagtggaagagtgtgcgggaaccaaggagctgctatgttctatgatgtgcctgaagaaacaggacctgtacaacaagttcaagggacgcgtgcggacggtttctcccagctccaagtccccctgggtggagtccgaatacctggattacctttttgaagtggagccggccccacctgtcctggtgctcacccagacggaggagatcctgagtgccaatgccacgtaccagctgcccccctgcatgcccccactgtttctgaagtatgaggtggcattttggggggggggggccggaaccaagaccctatttccagtcactccccatggccagccagtccagatcactctccagccagctgccagcgaacaccactgcctcagtgccagaaccatctacacgttcagtgtcccgaaatacagcaagttctctaagcccacctgcttcttgctggaggtcccagaagccaactgggctttcctggtgctgccatcgcttctgatactgctgttagtaattgccgcagggggtgtgatctggaagaccctcatggggaacccctggtttcagcgggcaaagatgccacgggccctggaactgaccagaggggtcaggccgacgcctcgagtcagggccccagccacccaacagacaagatggaagaaggaccttgcagaggacgaagaggaggaggatgaggaggacacagaagatggcgtcagcttccagccctacattgaaccaccttctttcctggggcaagagcaccaggctccagggcactcggaggctggtggggtggactcagggaggcccagggctcctctggtcccaagcgaaggctcctctgcttgggattcttcagacagaagctgggccagcactgtggactcctcctgggacagggctgggtcctctggctatttggctgagaaggggccaggccaagggccgggtggggatgggcaccaagaatctctcccaccacctgaattctccaaggactcgggtttcctggaagagctcccagaagataacctctcctcctgggccacctggggcaccttaccaccggagccgaatctggtccctgggggacccccagtttctcttcagacactgaccttctgctgggaaagcagccctgaggaggaagaggaggcgagggaatcagaaattgaggacagcgatgcgggcagctggggggctgagagcacccagaggaccgaggacaggggccggacattggggcattacatggccaggtga

SEQ ID NO: 14 - protein, 491 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLFLKYEVAFWGGGAGTKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 15 - DNA, 674 bp, Homo sapiens
  CDS (1)...(636)
  misc_feature (0)...(0): IL-28RA soluble variant
atggcggggcccgagcgctggggccccctgctcctgtgcctgctgcaggccgctccagggaggccccgtctggcccctccccagaatgtgacgctgctctcccagaacttcagcgtgtacctgacatggctcccagggcttggcaacccccaggatgtgacctattttgtggcctatcagagctctcccacccgtagacggtggcgcgaagtggaagagtgtgcgggaaccaaggagctgctatgttctatgatgtgcctgaagaaacaggacctgtacaacaagttcaagggacgcgtgcggacggtttctcccagctccaagtccccctgggtggagtccgaatacctggattacctttttgaagtggagccggccccacctgtcctggtgctcacccagacggaggagatcctgagtgccaatgccacgtaccagctgcccccctgcatgcccccactggatctgaagtatgaggtggcattctggaaggagggggccggaaacaaggtgggaagctcctttcctgcccccaggctaggcccgctcctccaccccttcttactcaggttcttctcaccctcccagcctgctcctgcacccctcctccaggaagtcttccctgtacactcctgacttctggcagtcagccctaataaaatctgatcaaagta

SEQ ID NO: 16 - protein, 211 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKVGSSFPAPRLGPLLHPFLLRFFSPSQPAPAPLLQEVFPVHS

SEQ ID NO: 17 - DNA, 734 bp, Homo sapiens
  sig_peptide (53)...(127)
  mat_peptide (128)...(655)
  CDS (53)...(655)
tgggtgacagcctcagagtgtttcttctgctgacaaagaccagagatcaggaatgaaactagacatgactggggactgcacgccagtgctggtgctgatggccgcagtgctgaccgtgactggagcagttcctgtcgccaggctccacggggctctcccggatgcaaggggctgccacatagcccagttcaagtccctgtctccacaggagctgcaggcctttaagagggccaaagatgccttagaagagtcgcttctgctgaaggactgcaggtgccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctgaccctcccaccagtcatgcaacctgagattttatttataaattagccacttgtcttaatttattgccacccagtcgctat

SEQ ID NO: 18 - protein, 200 aa, Homo sapiens
  SIGNAL (1)...(25)
MKLDMTGDCTPVLVLMAAVLTVTGAVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 19 - DNA, 856 bp, Homo sapiens
  sig_peptide (98)...(154)
  mat_peptide (155)...(700)
  CDS (98)...(700)
aattaccttttcactttacacacatcatcttggattgcccattttgcgtggctaaaaagcagagccatgccgctggggaagcagttgcgatttagccatggctgcagcttggaccgtggtgctggtgactttggtgctaggcttggccgtggcaggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgtgtctgagaacgtcaacccaccctgagtccacctgacaccccacaccttatttatgcgctgagccctactccttccttaatttatttcctctcaccctttatttatgaagctgcagccctgactgagacatagggctgagtttattgttttacttttatacattatgcacaaataaacaacaaggaattgga

SEQ ID NO: 20 - protein, 200 aa, Homo sapiens
  SIGNAL (1)...(19)
MAAAWTVVLVTLVLGLAVAGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 21 - DNA, 734 bp, Homo sapiens
  sig_peptide (53)...(127)
  mat_peptide (128)...(655)
  CDS (53)...(655)
tgggtgacagcctcagagtgtttcttctgctgacaaagaccagagatcaggaatgaaactagacatgaccggggactgcatgccagtgctggtgctgatggccgcagtgctgaccgtgactggagcagttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctgacccttccgccagtcatgcaacctgagattttatttataaattagccacttggcttaatttattgccacccagtcgctat

SEQ ID NO: 22 - protein, 200 aa, Homo sapiens
  SIGNAL (1)...(25)
MKLDMTGDCMPVLVLMAAVLTVTGAVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 23 - DNA, 528 bp, Artificial Sequence
  IL-28A mutant C48S
gttcctgtcgccaggctccacggggctctcccggatgcaaggggctgccacatagcccagttcaagtccctgtctccacaggagctgcaggcctttaagagggccaaagatgccttagaagagtcgcttctgctgaaggactccaggtgccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctga

SEQ ID NO: 24 - protein, 175 aa, Artificial Sequence
  IL-28A mutant C48S
VPVARLHGALPDARGCHIAQFKSLSPQELQAFKRAKDALEESLLLKDSRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 25 - DNA, 531 bp, Artificial Sequence
  met IL-28A mutant C49S
atggttcctgtcgccaggctccacggggctctcccggatgcaaggggctgccacatagcccagttcaagtccctgtctccacaggagctgcaggcctttaagagggccaaagatgccttagaagagtcgcttctgctgaaggactccaggtgccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctga

SEQ ID NO: 26 - protein, 176 aa, Artificial Sequence
  met IL-28A mutant C49S
MVPVARLHGALPDARGCHIAQFKSLSPQELQAFKRAKDALEESLLLKDSRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 27 - DNA, 528 bp, Artificial Sequence
  IL-28A mutant C50S
gttcctgtcgccaggctccacggggctctcccggatgcaaggggctgccacatagcccagttcaagtccctgtctccacaggagctgcaggcctttaagagggccaaagatgccttagaagagtcgcttctgctgaaggactgcaggtcccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctga

SEQ ID NO: 28 - protein, 175 aa, Artificial Sequence
  IL-28A mutant C50S
VPVARLHGALPDARGCHIAQFKSLSPQELQAFKRAKDALEESLLLKDCRSHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 29 - DNA, 531 bp, Artificial Sequence
  met IL-28A mutant C51S
atggttcctgtcgccaggctccacggggctctcccggatgcaaggggctgccacatagcccagttcaagtccctgtctccacaggagctgcaggcctttaagagggccaaagatgccttagaagagtcgcttctgctgaaggactgcaggtcccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctga

SEQ ID NO: 30 - protein, 176 aa, Artificial Sequence
  met IL-28A mutant C51S
MVPVARLHGALPDARGCHIAQFKSLSPQELQAFKRAKDALEESLLLKDCRSHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 31 - DNA, 546 bp, Artificial Sequence
  IL-29 mutant C171S
ggtccggttccgacctctaaaccaaccaccactggtaaaggttgccacatcggtcgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtaacctgtctctgcgtacctctacccatccggaatctacctaa

SEQ ID NO: 32 - protein, 181 aa, Artificial Sequence
  IL-29 mutant C171S
GPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLSLRTSTHPEST

SEQ ID NO: 33 - DNA, 549 bp, Artificial Sequence
  met IL-29 mutant C172S
atgggtccggttccgacctctaaaccaaccaccactggtaaaggttgccacatcggtcgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtaacctgtctctgcgtacctctacccatccggaatctacctaa

SEQ ID NO: 34 - protein, 182 aa, Artificial Sequence
  met IL-29 mutant C172S
MGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLSLRTSTHPEST

SEQ ID NO: 35 - DNA, 531 bp, Artificial Sequence
  met IL-28A
atggttcctgtcgccaggctccacggggctctcccggatgcaaggggctgccacatagcccagttcaagtccctgtctccacaggagctgcaggcctttaagagggccaaagatgccttagaagagtcgcttctgctgaaggactgcaggtgccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctga

SEQ ID NO: 36 - protein, 176 aa, Artificial Sequence
  met IL-28A
MVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 37 - DNA, 621 bp, Artificial Sequence
  met IL-29
atgggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgtgtctgagaacgtcaacccaccctgagtccacctgacaccccacaccttatttatgcgctgagccctactccttccttaatttatttcctctcaccctttatttatga

SEQ ID NO: 38 - protein, 182 aa, Artificial Sequence
  met IL-29
MGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 39 - DNA, 531 bp, Artificial Sequence
  met IL-28B
atggttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 40 - protein, 176 aa, Artificial Sequence
  met IL-28B
MVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 41 - DNA, 546 bp, Artificial Sequence
  IL-29 Cys15 mutant, Asn169
ggccctgtccccacttccaagcccaccacaactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaayctgtgtctgagaacgtcaacccaccctgagtccacctga

SEQ ID NO: 42 - protein, 181 aa, Artificial Sequence
  IL-29 Cys15 mutant, Asn169
GPVPTSKPTTTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 43 - DNA, 549 bp, Artificial Sequence
  Met IL-29 Cys16 mutant, Asn170
atgggccctgtccccacttccaagcccaccacaactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaayctgtgtctgagaacgtcaacccaccctgagtccacctga

SEQ ID NO: 44 - protein, 182 aa, Artificial Sequence
  Met IL-29 Cys16 mutant, Asn170
MGPVPTSKPTTTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 45 - DNA, 546 bp, Artificial Sequence
  IL-29 Cys15 mutant, Asp169
ggccctgtccccacttccaagcccaccacaactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgtgtctgagaacgtcaacccaccctgagtccacctga

SEQ ID NO: 46 - protein, 181 aa, Artificial Sequence
  IL-29 Cys15 mutant, Asp169
GPVPTSKPTTTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 47 - DNA, 549 bp, Artificial Sequence
  Met IL-29 Cys16 mutant, Asp170
atgggccctgtccccacttccaagcccaccacaactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgtgtctgagaacgtcaacccaccctgagtccacctga

SEQ ID NO: 48 - protein, 182 aa, Artificial Sequence
  Met IL-29 Cys16 mutant, Asp170
MGPVPTSKPTTTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 49 - DNA, 546 bp, Artificial Sequence
  IL-29 Asp169 Cys171 mutant
ggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgdnnctgagaacgtcaacccaccctgagtccacctga

SEQ ID NO: 50 - protein, 181 aa, Artificial Sequence
  IL-29 Asp169 Cys171 mutant
GPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLXLRTSTHPEST

SEQ ID NO: 51 - DNA, 549 bp, Artificial Sequence
  Met IL-29 Asp170 Cys172 mutant
atgggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgdnnctgagaacgtcaacccaccctgagtccacctga

SEQ ID NO: 52 - protein, 182 aa, Artificial Sequence
  Met IL-29 Asp170 Cys172 mutant
MGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLXLRTSTHPEST

SEQ ID NO: 53 - DNA, 546 bp, Artificial Sequence
  IL-29 Pro10 Asn169 Cys171 mutant
ggccctgtccccacttccaagcccaccccnactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 54 - protein, 181 aa, Artificial Sequence
  IL-29 Pro10 Asn169 Cys171 mutant
GPVPTSKPTPTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 55 - DNA, 549 bp, Artificial Sequence
  Met IL-29 Pro11 Asn170 Cys172 mutant
atgggccctgtccccacttccaagcccaccccnactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 56 - protein, 182 aa, Artificial Sequence
  Met IL-29 Pro11 Asn170 Cys172 mutant
MGPVPTSKPTPTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 57 - DNA, 546 bp, Artificial Sequence
  IL-29 Pro10 Cys15 mutant Asn169
ggccctgtccccacttccaagcccaccccnactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaayctgtgtctgagaacgtcaacccaccctgagtccacctga

SEQ ID NO: 58 - protein, 181 aa, Artificial Sequence
  IL-29 Pro10 Cys15 mutant Asn169
GPVPTSKPTPTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 59 - DNA, 549 bp, Artificial Sequence
  Met IL-29 Pro11 Cys16 mutant Asn170
atgggccctgtccccacttccaagcccaccccnactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaayctgtgtctgagaacgtcaacccaccctgagtccacctga

SEQ ID NO: 60 - protein, 182 aa, Artificial Sequence
  Met IL-29 Pro11 Cys16 mutant Asn170
MGPVPTSKPTPTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 61 - DNA, 546 bp, Artificial Sequence
  IL-29 Pro10 Asp169 Cys171 mutant
ggccctgtccccacttccaagcccaccccnactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgdnnctgagaacgtcaacccaccctgagtccacctga

SEQ ID NO: 62 - protein, 181 aa, Artificial Sequence
  IL-29 Pro10 Asp169 Cys171 mutant
GPVPTSKPTPTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLXLRTSTHPEST

SEQ ID NO: 63 - DNA, 549 bp, Artificial Sequence
  Met IL-29 Pro11 Asp170 Cys172 mutant
atgggccctgtccccacttccaagcccaccccnactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgdnnctgagaacgtcaacccaccctgagtccacctga

SEQ ID NO: 64 - protein, 182 aa, Artificial Sequence
  Met IL-29 Pro11 Asp170 Cys172 mutant
MGPVPTSKPTPTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLXLRTSTHPEST

SEQ ID NO: 65 - DNA, 546 bp, Artificial Sequence
  IL-29 Pro10 Cys15 mutant Asp169
ggccctgtccccacttccaagcccaccccnactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgtgtctgagaacgtcaacccaccctgagtccacctga

SEQ ID NO: 66 - protein, 181 aa, Artificial Sequence
  IL-29 Pro10 Cys15 mutant Asp169
GPVPTSKPTPTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 67 - DNA, 549 bp, Artificial Sequence
  Met IL-29 Pro11 Cys16 mutant Asp170
atgggccctgtccccacttccaagcccaccccnactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgtgtctgagaacgtcaacccaccctgagtccacctga

SEQ ID NO: 68 - protein, 182 aa, Artificial Sequence
  Met IL-29 Pro11 Cys16 mutant Asp170
MGPVPTSKPTPTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 69 - DNA, 546 bp, Artificial Sequence
  IL-29 Asp18 Asn169 Cys171 mutant
ggccctgtccccacttccaagcccaccacaactgggaagggctgccacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 70 - protein, 181 aa, Artificial Sequence
  IL-29 Asp18 Asn169 Cys171 mutant
GPVPTSKPTTTGKGCHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 71 - DNA, 549 bp, Artificial Sequence
  Met IL-29 Asp19 Asn170 Cys172 mutant
atgggccctgtccccacttccaagcccaccacaactgggaagggctgccacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 72 - protein, 182 aa, Artificial Sequence
  Met IL-29 Asp19 Asn170 Cys172 mutant
MGPVPTSKPTTTGKGCHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 73 - DNA, 546 bp, Artificial Sequence
  IL-29 Cys15 mutant Asp18 Asn169
ggccctgtccccacttccaagcccaccacaactgggaagggcdnncacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaayctgtgtctgagaacgtcaacccaccctgagtccacctga

SEQ ID NO: 74 - protein, 181 aa, Artificial Sequence
  IL-29 Cys15 mutant Asp18 Asn169
GPVPTSKPTTTGKGXHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 75 - DNA, 549 bp, Artificial Sequence
  Met IL-29 Cys16 mutant Asp19 Asn170
atgggccctgtccccacttccaagcccaccacaactgggaagggcdnncacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaayctgtgtctgagaacgtcaacccaccctgagtccacctga

SEQ ID NO: 76 - protein, 182 aa, Artificial Sequence
  Met IL-29 Cys16 mutant Asp19 Asn170
MGPVPTSKPTTTGKGXHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 77 - DNA, 546 bp, Artificial Sequence
  IL-29 Asp18 Asp169 Cys171 mutant
ggccctgtccccacttccaagcccaccacaactgggaagggctgccacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgdnnctgagaacgtcaacccaccctgagtccacctga

SEQ ID NO: 78 - protein, 181 aa, Artificial Sequence
  IL-29 Asp18 Asp169 Cys171 mutant
GPVPTSKPTTTGKGCHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLXLRTSTHPEST

SEQ ID NO: 79 - DNA, 549 bp, Artificial Sequence
  Met IL-29 Asp19 Asp170 Cys172 mutant
atgggccctgtccccacttccaagcccaccacaactgggaagggctgccacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgdnnctgagaacgtcaacccaccctgagtccacctga

SEQ ID NO: 80 - protein, 182 aa, Artificial Sequence
  Met IL-29 Asp19 Asp170 Cys172 mutant
MGPVPTSKPTTTGKGCHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLXLRTSTHPEST

SEQ ID NO: 81 - DNA, 546 bp, Artificial Sequence
  IL-29 Cys15 mutant Asp18 Asp169
ggccctgtccccacttccaagcccaccacaactgggaagggcdnncacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgtgtctgagaacgtcaacccaccctgagtccacctga

SEQ ID NO: 82 - protein, 181 aa, Artificial Sequence
  IL-29 Cys15 mutant Asp18 Asp169
GPVPTSKPTTTGKGXHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 83 - DNA, 549 bp, Artificial Sequence
  Met IL-29 Cys16 mutant Asp19 Asp170
atgggccctgtccccacttccaagcccaccacaactgggaagggcdnncacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgtgtctgagaacgtcaacccaccctgagtccacctga

SEQ ID NO: 84 - protein, 182 aa, Artificial Sequence
  Met IL-29 Cys16 mutant Asp19 Asp170
MGPVPTSKPTTTGKGXHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 85 - DNA, 528 bp, Artificial Sequence
  IL-28B Cys48 mutant
gttcctgtcgccaggctccgcggggctctcccggatgcaaggggctgccacatagcccagttcaagtccctgtctccacaggagctgcaggcctttaagagggccaaagatgccttagaagagtcgcttctgctgaaggacdnnaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 86 - protein, 175 aa, Artificial Sequence
  IL-28B Cys48 mutant
VPVARLRGALPDARGCHIAQFKSLSPQELQAFKRAKDALEESLLLKDXKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 87 - DNA, 531 bp, Artificial Sequence
  Met IL-28B Cys49 mutant
atggttcctgtcgccaggctccgcggggctctcccggatgcaaggggctgccacatagcccagttcaagtccctgtctccacaggagctgcaggcctttaagagggccaaagatgccttagaagagtcgcttctgctgaaggacdnnaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 88 - protein, 176 aa, Artificial Sequence
  Met IL-28B Cys49 mutant
MVPVARLRGALPDARGCHIAQFKSLSPQELQAFKRAKDALEESLLLKDXKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 89 - DNA, 528 bp, Artificial Sequence
  IL-28B Cys50 mutant
gttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagdnncgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 90 - protein, 175 aa, Artificial Sequence
  IL-28B Cys50 mutant
VPVARLRGALPDARGCHIAQFKSLSPQELQAFKRAKDALEESLLLKDCKXRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 91 - DNA, 531 bp, Artificial Sequence
  Met IL-28B Cys51 mutant
atggttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagdnncgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 92 - protein, 176 aa, Artificial Sequence
  Met IL-28B Cys51 mutant
MVPVARLRGALPDARGCHIAQFKSLSPQELQAFKRAKDALEESLLLKDCKXRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 93 - DNA, 528 bp, Artificial Sequence
  IL-28B Cys48 mutant T87S H135Y
gttcctgtcgccaggctccgcggggctctcccggatgcaaggggctgccacatagcccagttcaagtccctgtctccacaggagctgcaggcctttaagagggccaaagatgccttagaagagtcgcttctgctgaaggacdnnaagtgccgctcccgcctcttccccaggacctgggacctgaggcagctgcaggtgagggagcgccccgtggctttggaggctgagctggccctgacgctgaaggttctggaggccwsngctgacactgacccagccctgggggatgtcttggaccagccccttcacaccctgcaccatatcctctcccagctccgggcctgtatccagcctcagcccacggcagggcccaggacccggggccgcctccaccattggctgtaycggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 94 - protein, 175 aa, Artificial Sequence
  VARIANT (48)...(48): Xaa = Ser, Ala, Thr, Val, or Asn
VPVARLRGALPDARGCHIAQFKSLSPQELQAFKRAKDALEESLLLKDXKCRSRLFPRTWDLRQLQVRERPVALEAELALTLKVLEAXADTDPALGDVLDQPLHTLHHILSQLRACIQPQPTAGPRTRGRLHHWLYRLQEAPKKESPGCLEASVTFNLFRLLTRDLNCVASGDLCV

SEQ ID NO: 95 - DNA, 531 bp, Artificial Sequence
  Met IL-28B Cys49 mutant T88S H136Y
atggttcctgtcgccaggctccgcggggctctcccggatgcaaggggctgccacatagcccagttcaagtccctgtctccacaggagctgcaggcctttaagagggccaaagatgccttagaagagtcgcttctgctgaaggacdnnaagtgccgctcccgcctcttccccaggacctgggacctgaggcagctgcaggtgagggagcgccccgtggctttggaggctgagctggccctgacgctgaaggttctggaggccwsngctgacactgacccagccctgggggatgtcttggaccagccccttcacaccctgcaccatatcctctcccagctccgggcctgtatccagcctcagcccacggcagggcccaggacccggggccgcctccaccattggctgtaycggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 96 - protein, 176 aa, Artificial Sequence
  VARIANT (49)...(49): Xaa = Ser, Ala, Thr, Val, or Asn
MVPVARLRGALPDARGCHIAQFKSLSPQELQAFKRAKDALEESLLLKDXKCRSRLFPRTWDLRQLQVRERPVALEAELALTLKVLEAXADTDPALGDVLDQPLHTLHHILSQLRACIQPQPTAGPRTRGRLHHWLYRLQEAPKKESPGCLEASVTFNLFRLLTRDLNCVASGDLCV

SEQ ID NO: 97 - DNA, 528 bp, Artificial Sequence
  IL-28B Cys50 mutant T87S H135Y
gttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagdnncgctcccgcctcttccccaggacctgggacctgaggcagctgcaggtgagggagcgccccgtggctttggaggctgagctggccctgacgctgaaggttctggaggccwsngctgacactgacccagccctgggggatgtcttggaccagccccttcacaccctgcaccatatcctctcccagctccgggcctgtatccagcctcagcccacggcagggcccaggacccggggccgcctccaccattggctgtaycggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 98 - protein, 175 aa, Artificial Sequence
  VARIANT (50)...(50): Xaa = Ser, Ala, Thr, Val, or Asn
VPVARLRGALPDARGCHIAQFKSLSPQELQAFKRAKDALEESLLLKDCKXRSRLFPRTWDLRQLQVRERPVALEAELALTLKVLEAXADTDPALGDVLDQPLHTLHHILSQLRACIQPQPTAGPRTRGRLHHWLYRLQEAPKKESPGCLEASVTFNLFRLLTRDLNCVASGDLCV

SEQ ID NO: 99 - DNA, 531 bp, Artificial Sequence
  Met IL-28B Cys51 mutant T88S H136Y
atggttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagdnncgctcccgcctcttccccaggacctgggacctgaggcagctgcaggtgagggagcgccccgtggctttggaggctgagctggccctgacgctgaaggttctggaggccwsngctgacactgacccagccctgggggatgtcttggaccagccccttcacaccctgcaccatatcctctcccagctccgggcctgtatccagcctcagcccacggcagggcccaggacccggggccgcctccaccattggctgtaycggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 100 - protein, 176 aa, Artificial Sequence
  VARIANT (51)...(51): Xaa = Ser, Ala, Thr, Val, or Asn
MVPVARLRGALPDARGCHIAQFKSLSPQELQAFKRAKDALEESLLLKDCKXRSRLFPRTWDLRQLQVRERPVALEAELALTLKVLEAXADTDPALGDVLDQPLHTLHHILSQLRACIQPQPTAGPRTRGRLHHWLYRLQEAPKKESPGCLEASVTFNLFRLLTRDLNCVASGDLCV

SEQ ID NO: 101 - DNA, 45 bp, Artificial Sequence
  signal sequence
atggcngcngcntggacngtngtnytngtnacnytngtnytnggn

SEQ ID NO: 102 - protein, 15 aa, Artificial Sequence
  signal sequence
MAAAWTVVLVTLVLG

SEQ ID NO: 103 - DNA, 57 bp, Artificial Sequence
  signal sequence
atggcngcngcntggacngtngtnytngtnacnytngtnytnggnytngcngtngcn

SEQ ID NO: 104 - protein, 19 aa, Artificial Sequence
  signal sequence
MAAAWTVVLVTLVLGLAVA

SEQ ID NO: 105 - DNA, 63 bp, Artificial Sequence
  signal sequence
atggcngcngcntggacngtngtnytngtnacnytngtnytnggnytngcngtngcnggnccn

SEQ ID NO: 106 - protein, 21 aa, Artificial Sequence
  signal sequence
MAAAWTVVLVTLVLGLAVAGP

SEQ ID NO: 107 - DNA, 72 bp, Artificial Sequence
  signal sequence
atggcngcngcntggacngtngtnytngtnacnytngtnytnggnytngcngtngcnggnccngtnccnacn

SEQ ID NO: 108 - protein, 24 aa, Artificial Sequence
  signal sequence
MAAAWTVVLVTLVLGLAVAGPVPT

SEQ ID NO: 109 - DNA, 546 bp, Artificial Sequence
  IL-29 C171X
ggtccggttccgacctctaaaccaaccaccactggtaaaggttgccacatcggtcgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtaacctgdnnctgcgtacctctacccatccggaatctacctaa

SEQ ID NO: 110 - protein, 181 aa, Artificial Sequence
  IL-29 C171X
GPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 111 - DNA, 549 bp, Artificial Sequence
  Met IL-29 C172X
atgggtccggttccgacctctaaaccaaccaccactggtaaaggttgccacatcggtcgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtaacctgdnnctgcgtacctctacccatccggaatctacctaa

SEQ ID NO: 112 - protein, 182 aa, Artificial Sequence
  Met IL-29 C172X
MGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 113 - DNA, 543 bp, Artificial Sequence
  IL-29 C170X, truncated after N-terminal Methionine and Glycine
cctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 114 - protein, 180 aa, Artificial Sequence
  IL-29 C170X, truncated after N-terminal Methionine and Glycine
PVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 115 - DNA, 540 bp, Artificial Sequence
  IL-29 C169X, truncated after N-terminal Methionine, Glycine, and Proline
gtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 116 - protein, 179 aa, Artificial Sequence
  IL-29 C169X, truncated after N-terminal Methionine, Glycine, and Proline
VPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 117 - DNA, 537 bp, Artificial Sequence
  IL-29 C168X, truncated after N-terminal Methionine, Glycine, Proline, and Valine
cccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 118 - protein, 178 aa, Artificial Sequence
  IL-29 C168X, truncated after N-terminal Methionine, Glycine, Proline, and Valine
PTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 119 - DNA, 534 bp, Artificial Sequence
  IL-29 C167X, truncated after N-terminal Methionine, Glycine, Proline, Valine, and Proline
acttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 120 - protein, 177 aa, Artificial Sequence
  IL-29 C167X, truncated after N-terminal Methionine, Glycine, Proline, Valine, and Proline
TSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 121 - DNA, 531 bp, Artificial Sequence
  IL-29 C166X, truncated after N-terminal Methionine, Glycine, Proline, Valine, Proline, and Threonine
tccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 122 - protein, 176 aa, Artificial Sequence
  IL-29 C166X, truncated after N-terminal Methionine, Glycine, Proline, Valine, Proline, and Threonine
SKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 123 - DNA, 528 bp, Artificial Sequence
  IL-29 C165X, truncated after N-terminal Methionine, Glycine, Proline, Valine, Proline, Threonine, and Serine
aagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 124 - protein, 175 aa, Artificial Sequence
  IL-29 C165X, truncated after N-terminal Methionine, Glycine, Proline, Valine, Proline, Threonine, and Serine
KPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 125 - DNA, 552 bp, Artificial Sequence
  IL-29 Leu insert after N-terminal Met, C173X
atgytnggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 126 - protein, 183 aa, Artificial Sequence
  IL-29 Leu insert after N-terminal Met, C173X
MLGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 127 - DNA, 549 bp, Artificial Sequence
  IL-29 G2L C172X
atgytncctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 128 - protein, 182 aa, Artificial Sequence
  IL-29 G2L C172X
MLPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 129 - DNA, 552 bp, Artificial Sequence
  IL-29 Ile insert after N-terminal Met, C173X
atgathggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 130 - protein, 183 aa, Artificial Sequence
  IL-29 Ile insert after N-terminal Met, C173X
MIGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 131 - DNA, 549 bp, Artificial Sequence
  IL-29 G2I C172X
atgathcctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 132 - protein, 182 aa, Artificial Sequence
  IL-29 G2I C172X
MIPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 133 - DNA, 531 bp, Artificial Sequence
  IL-29 after N-terminal Met amino acid residues 2-7 deleted, C166X
atgaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 134 - protein, 176 aa, Artificial Sequence
  IL-29 after N-terminal Met amino acid residues 2-7 deleted, C166X
MKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 135 - DNA, 558 bp, Artificial Sequence
  IL-29 Glu, Ala, and Glu inserted after N-terminal Met, C175X
atggargcngarggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 136 - protein, 185 aa, Artificial Sequence
  IL-29 Glu, Ala, and Glu inserted after N-terminal Met, C175X
MEAEGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 137 - DNA, 528 bp, Artificial Sequence
  Human IL-28A C2 mutant for expression in E. coli
atggttccggttgctcgtctgcacggtgctctgccggacgctcgtggttgccacatcgctcagttcaaatctctgtctccgcaggaactgcaggctttcaaacgtgctaaagacgctctggaagaatctctgctgctgaaagacdnncgttgccactctcgtctgttcccgcgtacctgggacctgcgtcagctgcaggttcgtgaacgtccgatggctctggaagctgaactggctctgaccctgaaagttctggaagctaccgctgacaccgacccggctctggttgacgttctggaccagccgctgcacaccctgcaccacatcctgtctcagttccgtgcttgcatccagccgcagccgaccgctggtccgcgtacccgtggtcgtctgcaccactggctgtaccgtctgcaggaagctccgaaaaaagaatctccgggttgcctggaagcttctgttaccttcaacctgttccgtctgctgacccgtgacctgaactgcgttgcttctggtgacctgtgcgtt

SEQ ID NO: 138 - protein, 176 aa, Artificial Sequence
  Human IL-28A C2 mutant for expression in E. coli
MVPVARLHGALPDARGCHIAQFKSLSPQELQAFKRAKDALEESLLLKDXRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 139 - DNA, 528 bp, Artificial Sequence
  Human IL-28A C3 mutant for expression in E. coli
atggttccggttgctcgtctgcacggtgctctgccggacgctcgtggttgccacatcgctcagttcaaatctctgtctccgcaggaactgcaggctttcaaacgtgctaaagacgctctggaagaatctctgctgctgaaagactgccgtdnncactctcgtctgttcccgcgtacctgggacctgcgtcagctgcaggttcgtgaacgtccgatggctctggaagctgaactggctctgaccctgaaagttctggaagctaccgctgacaccgacccggctctggttgacgttctggaccagccgctgcacaccctgcaccacatcctgtctcagttccgtgcttgcatccagccgcagccgaccgctggtccgcgtacccgtggtcgtctgcaccactggctgtaccgtctgcaggaagctccgaaaaaagaatctccgggttgcctggaagcttctgttaccttcaacctgttccgtctgctgacccgtgacctgaactgcgttgcttctggtgacctgtgcgtt

SEQ ID NO: 140 - protein, 176 aa, Artificial Sequence
  Human IL-28A C3 mutant for expression in E. coli
MVPVARLHGALPDARGCHIAQFKSLSPQELQAFKRAKDALEESLLLKDCRXHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 141 - DNA, 528 bp, Artificial Sequence
  Human IL-28B C2 mutant for expression in E. coli
atggttccggttgctcgtctgcgtggtgctctgccggacgctcgtggttgccacatcgctcagttcaaatctctgtctccgcaggaactgcaggctttcaaacgtgctaaagacgctctggaagaatctctgctgctgaaagacdnnaaatgccgttctcgtctgttcccgcgtacctgggacctgcgtcagctgcaggttcgtgaacgtccggttgctctggaagctgaactggctctgaccctgaaagttctggaagctwsngctgacaccgacccggctctgggtgacgttctggaccagccgctgcacaccctgcaccacatcctgtctcagctgcgtgcttgcatccagccgcagccgaccgctggtccgcgtacccgtggtcgtctgcaccactggctgyaycgtctgcaggaagctccgaaaaaagaatctccgggttgcctggaagcttctgttaccttcaacctgttccgtctgctgacccgtgacctgaactgcgttgcttctggtgacctgtgcgtt

SEQ ID NO: 142 - protein, 176 aa, Artificial Sequence
  Human IL-28B C2 mutant for expression in E. coli
MVPVARLRGALPDARGCHIAQFKSLSPQELQAFKRAKDALEESLLLKDXKCRSRLFPRTWDLRQLQVRERPVALEAELALTLKVLEAXADTDPALGDVLDQPLHTLHHILSQLRACIQPQPTAGPRTRGRLHHWLXRLQEAPKKESPGCLEASVTFNLFRLLTRDLNCVASGDLCV

SEQ ID NO: 143 - DNA, 528 bp, Artificial Sequence
  Human IL-28B C3 mutant for expression in E. coli
atggttccggttgctcgtctgcgtggtgctctgccggacgctcgtggttgccacatcgctcagttcaaatctctgtctccgcaggaactgcaggctttcaaacgtgctaaagacgctctggaagaatctctgctgctgaaagactgcaaadnncgttctcgtctgttcccgcgtacctgggacctgcgtcagctgcaggttcgtgaacgtccggttgctctggaagctgaactggctctgaccctgaaagttctggaagctwsngctgacaccgacccggctctgggtgacgttctggaccagccgctgcacaccctgcaccacatcctgtctcagctgcgtgcttgcatccagccgcagccgaccgctggtccgcgtacccgtggtcgtctgcaccactggctgyaycgtctgcaggaagctccgaaaaaagaatctccgggttgcctggaagcttctgttaccttcaacctgttccgtctgctgacccgtgacctgaactgcgttgcttctggtgacctgtgcgtt

SEQ ID NO: 144 - protein, 176 aa, Artificial Sequence
  Human IL-28B C3 mutant for expression in E. coli
MVPVARLRGALPDARGCHIAQFKSLSPQELQAFKRAKDALEESLLLKDCKXRSRLFPRTWDLRQLQVRERPVALEAELALTLKVLEAXADTDPALGDVLDQPLHTLHHILSQLRACIQPQPTAGPRTRGRLHHWLXRLQEAPKKESPGCLEASVTFNLFRLLTRDLNCVASGDLCV

SEQ ID NO: 145 - DNA, 549 bp, Artificial Sequence
  Human IL-29 C1 mutant for expression in E. coli
atgggtccggttccgacctctaaaccaaccmcnactggtaaaggtdnncacatcgrncgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtrayctgtgcctgcgtacctctacccatccggaatctacctaa

SEQ ID NO: 146 - protein, 182 aa, Artificial Sequence
  Human IL-29 C1 mutant for expression in E. coli
MGPVPTSKPTXTGKGXHIXRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGXLCLRTSTHPEST

SEQ ID NO: 147 - DNA, 549 bp, Artificial Sequence
  Human IL-29 C5 mutant for expression in E. coli
atgggtccggttccgacctctaaaccaaccmcnactggtaaaggttgccacatcgrncgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtrayctgdnnctgcgtacctctacccatccggaatctacctaa

SEQ ID NO: 148 - protein, 182 aa, Artificial Sequence
  Human IL-29 C5 mutant for expression in E. coli
MGPVPTSKPTXTGKGCHIXRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGXLXLRTSTHPEST

SEQ ID NO: 149 - DNA, 531 bp, Artificial Sequence
  Human IL-29 d2/7 C5 mutant for expression in E. coli
atgaaaccaaccaccactggtaaaggttgccacatcggtcgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtaacctgdnnctgcgtacctctacccatccggaatctacctaa

SEQ ID NO: 150 - protein, 176 aa, Artificial Sequence; identical to SEQ ID NO: 134
  Human IL-29 d2/7 C5 mutant for expression in E. coli

(19) United States
(12) Patent Application Publication
(10) Pub. No.: US 2011/0182852 A1
(43) Pub. Date: Jul. 28, 2011

(54) IL28 AND IL29 TRUNCATED CYSTEINE MUTANTS AND ANTIVIRAL METHODS OF USING SAME

(75) Inventor: PAUL O. SHEPPARD, GRANITE FALLS, WA (US)

(73) Assignees: ZYMOGENETICS, INC.; ZYMOGENETICS LLC

(21) Appl. No.: 13/042,083

(22) Filed: Mar. 7, 2011

Related U.S. Application Data
(63) Continuation of application No. 12/352,454, filed on Jan. 12, 2009.
Application No. 12/352,454 is a division of application No. 11/458,945, filed on Jul. 20, 2006, now abandoned.
(60) Provisional application No. 60/700,905, filed on Jul. 20, 2005.

(51) Int. Cl. A61K 38/20; A61P 31/14

(52) U.S. Cl. 424/85.2

(57) ABSTRACT

IL-28A, IL-28B, IL-29, and certain mutants thereof have been shown to have antiviral activity on a spectrum of viral species. Of particular interest is the antiviral activity demonstrated on viruses that infect liver, such as hepatitis B virus and hepatitis C virus. In addition, IL-28A, IL-28B, IL-29, and mutants thereof do not exhibit some of the antiproliferative activity on hematopoietic cells that is observed with interferon treatment. Without the immunosuppressive effects accompanying interferon treatment, IL-28A, IL-28B, and IL-29 will be useful in treating immunocompromised patients for viral infections.

### CROSS-REFERENCE TO RELATED APPLICATIONS

The present application is a continuation of U.S. patent application Ser. No. 12/352,454, filed Jan. 12, 2009, which is a divisional of U.S. patent application Ser. No. 11/458,945, filed Jul. 20, 2006, now abandoned, which claims the benefit of U.S. Patent Application Ser. No. 60/700,905, filed Jul. 20, 2005, which are all herein incorporated by reference.

### BACKGROUND OF THE INVENTION

Strategies for treating infectious disease often focus on ways to enhance immunity. For instance, the most common method for treating viral infection involves prophylactic vaccines that induce immune-based memory responses. Another method for treating viral infection includes passive immunization via immunoglobulin therapy (Meissner, J. Pediatr. 124:S17-21, 1994). Administration of Interferon alpha (IFN-α) is another method for treating viral infections such as genital warts (Reichman et al., Ann. Intern. Med. 108:675-9, 1988) and chronic viral infections like hepatitis C virus (HCV) (Davis et al., New Engl. J. Med. 339:1493-9, 1998) and hepatitis B virus (HBV). For instance, IFN-α and IFN-β are critical for inhibiting virus replication (reviewed by Vilcek et al., (Eds.), Interferons and other cytokines. In Fields Fundamental Virology., 3^{rd} ed., Lippincott-Raven Publishers Philadelphia, Pa., 1996, pages 341-365). In response to viral infection, CD4+ T cells become activated and initiate a T-helper type I (TH1) response and the subsequent cascade required for cell-mediated immunity. That is, following their expansion by specific growth factors like the cytokine IL-2, T-helper cells stimulate antigen-specific CD8+ T-cells, macrophages, and NK cells to kill virally infected host cells. Although oftentimes efficacious, these methods have limitations in clinical use. For instance, many viral infections are not amenable to vaccine development, nor are they treatable with antibodies alone. In addition, IFN's are not extremely effective and they can cause significant toxicities; thus, there is a need for improved therapies.

Not all viruses and viral diseases are treated identically because factors, such as whether an infection is acute or chronic and the patient's underlying health, influence the type of treatment that is recommended. Generally, treatment of acute infections in immunocompetent patients should reduce the disease's severity, decrease complications, and decrease the rate of transmission. Safety, cost, and convenience are essential considerations in recommending an acute antiviral agent. Treatments for chronic infections should prevent viral damage to organs such as liver, lungs, heart, central nervous system, and gastrointestinal system, making efficacy the primary consideration.

Chronic hepatitis is one of the most common and severe viral infections of humans worldwide belonging to the Hepadnaviridae family of viruses. Infected individuals are at high risk for developing liver cirrhosis, and eventually, hepatic cancer. Chronic hepatitis is characterized as an inflammatory liver disease continuing for at least six months without improvement. The majority of patients suffering from chronic hepatitis are infected with either chronic HBV, HCV or are suffering from autoimmune disease. The prevalence of HCV infection in the general population exceeds 1% in the United States, Japan, China and Southeast Asia.

Chronic HCV can progress to cirrhosis and extensive necrosis of the liver. Although chronic HCV is often associated with deposition of type I collagen leading to hepatic fibrosis, the mechanisms of fibrogenesis remain unknown. Liver (hepatic) fibrosis occurs as a part of the wound-healing response to chronic liver injury. Fibrosis occurs as a complication of haemochromatosis, Wilson's disease, alcoholism, schistosomiasis, viral hepatitis, bile duct obstruction, toxin exposure, and metabolic disorders. This formation of scar tissue is believed to represent an attempt by the body to encapsulate the injured tissue. Liver fibrosis is characterized by the accumulation of extracellular matrix that can be distinguished qualitatively from that in normal liver. Left unchecked, hepatic fibrosis progresses to cirrhosis (defined by the presence of encapsulated nodules), liver failure, and death.

There are few effective treatments for hepatitis. For example, treatment of autoimmune chronic hepatitis is generally limited to immunosuppressive treatment with corticosteroids. For the treatment of HBV and HCV, the FDA has approved administration of recombinant IFN-α. However, IFN-α is associated with a number of dose-dependent adverse effects, including thrombocytopenia, leukopenia, bacterial infections, and influenza-like symptoms. Other agents used to treat chronic HBV or HCV include the nucleoside analog RIBAVIRIN™ and ursodeoxycholic acid; however, neither has been shown to be very effective. RIBAVIRIN™+IFN combination therapy for results in 47% rate of sustained viral clearance (Lanford, R. E. and Bigger, C. Virology 293: 1-9 (2002). (See Medicine, (D. C. Dale and D. D. Federman, eds.) (Scientific American, Inc., New York), 4:VIII:1-8 (1995)).

Respiratory syncytial virus is the major cause of pneumonia and bronchiolitis in infancy. RSV infects more than half of infants during their first year of exposure, and nearly all are infected after a second year. During seasonal epidemics most infants, children, and adults are at risk for infection or reinfection. Other groups at risk for serious RSV infections include premature infants, immune compromised children and adults, and the elderly. Symptoms of RSV infection range from a mild cold to severe bronchiolitis and pneumonia. Respiratory syncytial virus has also been associated with acute otitis media and RSV can be recovered from middle ear fluid. Herpes simplex virus-1 (HSV-1) and herpes simplex virus-2 (HSV-2) may be either lytic or latent, and are the causative agents in cold sores (HSV-1) and genital herpes, typically associated with lesions in the region of the eyes, mouth, and genitals (HSV-2). These viruses are a few examples of the many viruses that infect humans for which there are few adequate treatments available once infection has occurred.

The demonstrated activities of the IL-28 and IL-29 cytokine family provide methods for treating specific viral infections, for example, liver specific viral infections. The activity of IL-28 and IL-29 also demonstrate that these cytokines provide methods for treating immunocompromised patients. The methods for these and other uses should be apparent to those skilled in the art from the teachings herein.

### DESCRIPTION OF THE INVENTION

Definitions

In the description that follows, a number of terms are used extensively. The following definitions are provided to facilitate understanding of the invention.

Unless otherwise specified, “a,” “an,” “the,” and “at least one” are used interchangeably and mean one or more than one.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “level” when referring to immune cells, such as NK cells, T cells, in particular cytotoxic T cells, B cells and the like, an increased level is either increased number of cells or enhanced activity of cell function.

The term “level” when referring to viral infections refers to a change in the level of viral infection and includes, but is not limited to, a change in the level of CTLs or NK cells (as described above), a decrease in viral load, an increase antiviral antibody titer, decrease in serological levels of alanine aminotransferase, or improvement as determined by histological examination of a target tissue or organ. Determination of whether these changes in level are significant differences or changes is well within the skill of one in the art.

The term “operably linked”, when referring to DNA segments, indicates that the segments are arranged so that they function in concert for their intended purposes, e.g., transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

“zcyto20”, “zcyto21”, “zcyto22” are the previous designations for human IL-28A, IL-29, and IL-28B, respectively and are used interchangeably herein. IL-28A polypeptides of the present invention are shown in SEQ ID NOs:2, 18, 24, 26, 28, 30, 36, 138 and 140, which are encoded by polynucleotide sequences as shown in SEQ ID NOs:1, 17, 23, 25, 27, 29, 35, 137 and 139, respectively. IL-28B polypeptides of the present invention are shown in SEQ ID NOs:6, 22, 40, 86, 88, 90, 92, 94, 96, 98, 100, 142 and 144, which are encoded by polynucleotide sequences as shown in SEQ ID NOs:5, 21, 39, 85, 87, 89, 91, 93, 95, 97, 99, 141 and 143, respectively. IL-29 polypeptides of the present invention are shown in SEQ ID NOs:4, 20, 32, 34, 38, 42, 44, 46, 48, 50, 52, 54, 56, 58, 60, 62, 64, 66, 68, 70, 72, 74, 76, 78, 80, 82, 84, 110, 112, 114, 116, 118, 120, 122, 124, 126, 128, 130, 132, 134, 136, 146, 148 and 150, which are encoded by polynucleotide sequences as shown in SEQ ID NOs:3, 19, 31, 33, 37, 41, 43, 45, 47, 49, 51, 53, 55, 57, 59, 61, 63, 65, 67, 69, 71, 73, 75, 77, 79, 81, 83, 109, 111, 113, 115, 117, 119, 121, 123, 125, 127, 129, 131, 133, 135, 145, 147 and 149, respectively.

“zcyto24” and “zcyto25” are the previous designations for mouse IL-28A and IL-28B, and are shown in SEQ ID NOs:7, 8, 9, 10, respectively. The polynucleotide and polypeptides are fully described in PCT application WO 02/086087 commonly assigned to ZymoGenetics, Inc., incorporated herein by reference.

“zcytor19” is the previous designation for IL-28 receptor α-subunit, and is shown in SEQ ID NOs:11, 12, 13, 14, 15, 16. The polynucleotides and polypeptides are described in PCT application WO 02/20569 on behalf of Schering, Inc., and WO 02/44209 assigned to ZymoGenetics, Inc and incorporated herein by reference. “IL-28 receptor” denotes the IL-28 α-subunit and CRF2-4 subunit forming a heterodimeric receptor.

In one aspect, the present invention provides methods for treating viral infections comprising administering to a mammal with a viral infection a therapeutically effective amount of a polypeptide comprising an amino acid sequence that has at least 95% identity to amino acid residues of SEQ ID NO:134, wherein after administration of the polypeptide the viral infection level is reduced. In other embodiments, the methods comprise administering a polypeptide comprising an amino acid sequence selected from the group of SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and 150. The polypeptide may optionally comprise at least 15, at least 30, at least 45, or at least 60 sequential amino acids of an amino acid sequence selected from the group of SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and 150. In another aspect, the viral infection can optionally cause liver inflammation, wherein administering a therapeutically effective amount of a polypeptide reduces the liver inflammation. In other embodiments, the polypeptide is conjugated to a polyalkyl oxide moiety, such as polyethylene glycol (PEG), or F_{c}, or human albumin. The PEG may be N-terminally conjugated to the polypeptide and may comprise, for instance, a 20 kD or 30 kD monomethoxy-PEG propionaldehyde. In another embodiment, a reduction in the viral infection level is measured as a decrease in viral load, an increase in antiviral antibodies, a decrease in serological levels of alanine aminotransferase or histological improvement. In another embodiment, the mammal is a human. In another embodiment, the present invention provides that the viral infection is a hepatitis B viral infection and/or a hepatitis C viral infection. In another embodiment, the polypeptide may be given prior to, concurrent with, or subsequent to, at least one additional antiviral agent selected from the group of Interferon alpha, Interferon beta, Interferon gamma, Interferon omega, protease inhibitor, RNA or DNA polymerase inhibitor, nucleoside analog, antisense inhibitor, and combinations thereof. The polypeptide may be administered intravenously, intraperitoneally, intrathecally, intramuscularly, subcutaneously, orally, intranasally, or by inhalation.

In one aspect, the present invention provides methods for treating viral infections comprising administering to a mammal with a viral infection a therapeutically effective amount of a composition comprising a polypeptide comprising an amino acid sequence that has at least 95% identity to amino acid residues of SEQ ID NO:134, and a pharmaceutically acceptable vehicle, wherein after administration of the composition the viral infection level is reduced. In other embodiments, the methods comprise administering composition comprising the polypeptide comprising an amino acid sequence as shown in SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and/or 150. The polypeptide may optionally comprise at least 15, at least 30, at least 45, or at least 60 sequential amino acids of an amino acid sequence as shown in SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and/or 150. In other embodiments, the polypeptide is conjugated to a polyalkyl oxide moiety, such as PEG, or F_{c}, or human albumin. The PEG may be N-terminally conjugated to the polypeptide and may comprise, for instance, a 20 kD or 30 kD monomethoxy-PEG propionaldehyde. In another embodiment, a reduction in the viral infection level is measured as a decrease in viral load, an increase in antiviral antibodies, a decrease in serological levels of alanine aminotransferase or histological improvement. In another embodiment, the mammal is a human. In another embodiment, the present invention provides that the viral infection is a hepatitis B virus infection or a hepatitis C virus infection. In another embodiment, the composition may further include or, be given prior to or, be given concurrent with, or be given subsequent to, at least one additional antiviral agent selected from the group of Interferon alpha, Interferon beta, Interferon gamma, Interferon omega, protease inhibitor, RNA or DNA polymerase inhibitor, nucleoside analog, antisense inhibitor, and combinations thereof. The composition may be administered intravenously, intraperitoneally, intrathecally, intramuscularly, subcutaneously, orally, intranasally, or by inhalation.

In one aspect, the present invention provides methods for treating viral infections comprising administering to a mammal with a viral infection causing liver inflammation a therapeutically effective amount of a composition comprising a polypeptide comprising an amino acid sequence that has at least 95% identity to amino acid residues of SEQ ID NO:134, and a pharmaceutically acceptable vehicle, wherein after administration of the composition the viral infection level or liver inflammation is reduced. In other embodiments, the methods comprise administering composition comprising the polypeptide comprising an amino acid sequence as shown in SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and/or 150. The polypeptide may optionally comprise at least 15, at least 30, at least 45, or at least 60 sequential amino acids of an amino acid sequence as shown in SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and/or 150. In other embodiments, the polypeptide is conjugated to a polyalkyl oxide moiety, such as PEG, or F_{c}, or human albumin. The PEG may be N-terminally conjugated to the polypeptide and may comprise, for instance, a 20 kD or 30 kD monomethoxy-PEG propionaldehyde. In another embodiment, a reduction in the viral infection level is measured as a decrease in viral load, an increase in antiviral antibodies, a decrease in serological levels of alanine aminotransferase or histological improvement. In another embodiment, the mammal is a human. In another embodiment, the present invention provides that the viral infection is a hepatitis B virus infection or a hepatitis C virus infection. In another embodiment, the composition may further include or, be given prior to or, be given concurrent with, or be given subsequent to, at least one additional antiviral agent selected from the group of Interferon alpha, Interferon beta, Interferon gamma, Interferon omega, protease inhibitor, RNA or DNA polymerase inhibitor, nucleoside analog, antisense inhibitor, and combinations thereof. The composition may be administered intravenously, intraperitoneally, intrathecally, intramuscularly, subcutaneously, orally, intranasally, or by inhalation.

In another aspect, the present invention provides methods for treating liver inflammation comprising administering to a mammal in need thereof a therapeutically effective amount of a polypeptide comprising an amino acid sequence that has at least 95% identity to amino acid residues of SEQ ID NO:134, wherein after administration of the polypeptide the liver inflammation is reduced. In one embodiment, the invention provides that the polypeptide comprises an amino acid sequence as shown in SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and/or 150. The polypeptide may optionally comprise at least 15, at least 30, at least 45, or at least 60 sequential amino acids of an amino acid sequence as shown in SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and/or 150. In another embodiment, the polypeptide is conjugated to a polyalkyl oxide moiety, such as PEG, or human albumin, or F_{c}. The PEG may be N-terminally conjugated to the polypeptide and may comprise, for instance, a 20 kD or 30 kD monomethoxy-PEG propionaldehyde. In another embodiment, the present invention provides that the reduction in the liver inflammation is measured as a decrease in serological level of alanine aminotransferase or histological improvement. In another embodiment, the mammal is a human. In another embodiment, the liver inflammation is associated with a hepatitis C viral infection or a hepatitis B viral infection. In another embodiment, the polypeptide may be given prior to, concurrent with, or subsequent to, at least one additional antiviral agent selected from the group of Interferon alpha, Interferon beta, Interferon gamma, Interferon omega, protease inhibitor, RNA or DNA polymerase inhibitor, nucleoside analog, antisense inhibitor, and combinations thereof. The polypeptide may be administered intravenously, intraperitoneally, intrathecally, intramuscularly, subcutaneously, orally, intranasally, or by inhalation.

In another aspect, the present invention provides methods for treating liver inflammation comprising administering to a mammal in need thereof a therapeutically effective amount of a composition comprising a polypeptide comprising an amino acid sequence that has at least 95% identity to amino acid residues of SEQ ID NO:134, wherein after administration of the polypeptide the liver inflammation is reduced. In one embodiment, the invention provides that the polypeptide comprises an amino acid sequence as shown in SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and/or 150. The polypeptide may optionally comprise at least 15, at least 30, at least 45, or at least 60 sequential amino acids of an amino acid sequence as shown in SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and/or 150. In another embodiment, the polypeptide is conjugated to a polyalkyl oxide moiety, such as PEG, or human albumin, or F_{c}. The PEG may be N-terminally conjugated to the polypeptide and may comprise, for instance, a 20 kD or 30 kD monomethoxy-PEG propionaldehyde. In another embodiment, the present invention provides that the reduction in the liver inflammation is measured as a decrease in serological level of alanine aminotransferase or histological improvement. In another embodiment, the mammal is a human. In another embodiment, the liver inflammation is associated with a hepatitis C virus infection or a hepatitis B virus infection. In another embodiment, the composition may further include or, be given prior to or, be given concurrent with, or be given subsequent to, at least one additional antiviral agent selected from the group of Interferon alpha, Interferon beta, Interferon gamma, Interferon omega, protease inhibitor, RNA or DNA polymerase inhibitor, nucleoside analog, antisense inhibitor, and combinations thereof. The composition may be administered intravenously, intraperitoneally, intrathecally, intramuscularly, subcutaneously, orally, intranasally, or by inhalation.

In another aspect, the present invention provides methods of treating a viral infection comprising administering to an immunocompromised mammal with an viral infection a therapeutically effective amount of a polypeptide comprising an amino acid sequence that has at least 95% identity to amino acid residues of SEQ ID NO:134, wherein after administration of the polypeptide the viral infection is reduced. In another embodiment, the polypeptide comprises an amino acid sequence as shown in SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and/or 150. The polypeptide may optionally comprise at least 15, at least 30, at least 45, or at least 60 sequential amino acids of an amino acid sequence as shown in SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and/or 150. In another embodiment, the polypeptide is conjugated to a polyalkyl oxide moiety, such as PEG, or human albumin, or F_{c}. The PEG may be N-terminally conjugated to the polypeptide and may comprise, for instance, a 20 kD or 30 kD monomethoxy-PEG propionaldehyde. In another embodiment, a reduction in the viral infection level is measured as a decrease in viral load, an increase in antiviral antibodies, a decrease in serological levels of alanine aminotransferase or histological improvement. In another embodiment, the mammal is a human. In another embodiment, the present invention provides that the viral infection is a hepatitis B virus infection or a hepatitis C virus infection. In another embodiment, the polypeptide may be given prior to, concurrent with, or subsequent to, at least one additional antiviral agent selected from the group of Interferon alpha, Interferon beta, Interferon gamma, Interferon omega, protease inhibitor, RNA or DNA polymerase inhibitor, nucleoside analog, antisense inhibitor, and combinations thereof. The polypeptide may be administered intravenously, intraperitoneally, intrathecally, intramuscularly, subcutaneously, orally, intranasally, or by inhalation.

In another aspect, the present invention provides methods of treating liver inflammation comprising administering to an immunocompromised mammal with liver inflammation a therapeutically effective amount of a polypeptide comprising an amino acid sequence that has at least 95% identity to amino acid residues of SEQ ID NO:134, wherein after administration of the polypeptide the liver inflammation is reduced. In another embodiment, the polypeptide comprises an amino acid sequence as shown in SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and/or 150. The polypeptide may optionally comprise at least 15, at least 30, at least 45, or at least 60 sequential amino acids of an amino acid sequence as shown in SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and/or 150. In another embodiment, the polypeptide is conjugated to a polyalkyl oxide moiety, such as PEG, or human albumin, or F_{c}. The PEG may be N-terminally conjugated to the polypeptide and may comprise, for instance, a 20 kD or 30 kD monomethoxy-PEG propionaldehyde. In another embodiment, a reduction in the liver inflammation level is measured as a decrease in serological levels of alanine aminotransferase or histological improvement. In another embodiment, the mammal is a human. In another embodiment, the present invention provides that the viral infection is a hepatitis B virus infection or a hepatitis C virus infection. In another embodiment, the mammal is a human. In another embodiment, the present invention provides that the viral infection is a hepatitis B virus infection or a hepatitis C virus infection. In another embodiment, the polypeptide may be given prior to, concurrent with, or subsequent to, at least one additional antiviral agent selected from the group of Interferon alpha, Interferon beta, Interferon gamma, Interferon omega, protease inhibitor, RNA or DNA polymerase inhibitor, nucleoside analog, antisense inhibitor, and combinations thereof. The polypeptide may be administered intravenously, intraperitoneally, intrathecally, intramuscularly, subcutaneously, orally, intranasally, or by inhalation.

The discovery of a new family of interferon-like molecules was previously described in PCT applications, PCT/US01/21087 and PCT/US02/12887, and Sheppard et al., Nature Immunol 4:63-68, 2003; U.S. Patent Application Ser. Nos. 60/493,194 and 60/551,841; all incorporated by reference herein. This new family includes molecules designated zcyto20, zcyto21, zcyto22, zcyto24, zcyto25, where zcyto20, 21, and 22 are human sequences, and zcyto24 and 25 are mouse sequences. HUGO designations have been assigned to the interferon-like proteins. Zcyto20 has been designated IL-28A, zycto22 has been designated IL-28B, zycto21 has been designated IL-29. Kotenko et al., Nature Immunol. 4:69-77, 2003, have identified IL-28A as IFNλ2, IL-28B as IFNλ3, and IL-29 as IFNλ1. The receptor for these proteins, originally designated zcytor19 (SEQ ID NOs:11 and 12), has been designated as IL-28RA by HUGO. When referring to “IL-28”, the term shall mean both IL-28A and IL-28B.

The present invention provides methods for using IL-28 and IL-29 as an antiviral agent in a broad spectrum of viral infections. In certain embodiments, the methods include using IL-28 and IL-29 in viral infections that are specific for liver, such as hepatitis. Furthermore, data indicate that IL-28 and IL-29 exhibit these antiviral activities without some of the toxicities associated with the use of IFN therapy for viral infection. One of the toxicities related to type I interferon therapy is myelosuppression. This is due to type I interferons suppression of bone marrow progenitor cells. Because IL-29 does not significantly suppress bone marrow cell expansion or B cell proliferation as is seen with IFN-α, IL-29 will have less toxicity associated with treatment. Similar results would be expected with IL-28A and IL-28B.

IFN-α may be contraindicated in some patients, particularly when doses sufficient for efficacy have some toxicity or myelosuppressive effects. Examples of patients for which IFN is contraindicated can include (1) patients given previous immunosuppressive medication, (2) patients with HIV or hemophilia, (3) patients who are pregnant, (4) patients with a cytopenia, such as leukocyte deficiency, neutropenia, thrombocytopenia, and (5) patients exhibiting increased levels of serum liver enzymes. Moreover, IFN therapy is associated with symptoms that are characterized by nausea, vomiting, diarrhea and anorexia. The result being that some populations of patients will not tolerate IFN therapy, and IL-28A, IL-28B, and IL-29 can provide an alternative therapy for some of those patients.

The methods of the present invention comprise administering a therapeutically effective amount of an IL-28A, IL-28B, and/or IL-29 polypeptide of the present invention that have retained some biological activity associated with IL-28A, IL-28B or IL-29, alone or in combination with other biologics or pharmaceuticals. The present invention provides methods of treating a mammal with a chronic or acute viral infection, causing liver inflammation, thereby reducing the viral infection or liver inflammation. In another aspect, the present invention provides methods of treating liver specific diseases, in particular liver disease where viral infection is in part an etiologic agent. These methods are based on the discovery that IL-28 and IL-29 have antiviral activity on hepatic cells.

As stated above, the methods of the present invention provide administering a therapeutically effective amount of an IL-28A, IL-28B, and/or IL-29 polypeptide of the present invention that have retained some biological activity associated with IL-28A, IL-28B or IL-29, alone or in combination with other biologics or pharmaceuticals. The present invention provides methods of treatment of a mammal with a viral infection selected from the group consisting of hepatitis A, hepatitis B, hepatitis C, and hepatitis D. Other aspects of the present invention provide methods for using IL-28 or IL-29 as an antiviral agent in viral infections selected from the group consisting of respiratory syncytial virus, herpes virus, Epstein-Barr virus, norovirus, influenza virus (e.g., avian influenza A virus, for instance the H5N1 virus), adenovirus, parainfluenza virus, rhino virus, coxsackie virus, vaccinia virus, west nile virus, severe acute respiratory syndrome, dengue virus, venezuelan equine encephalitis virus, pichinde virus and polio virus. In certain embodiments, the mammal can have either a chronic or acute viral infection.

In another aspect, the methods of the present invention also include a method of treating a viral infection comprising administering a therapeutically effective amount of IL-28A, IL-28B, and/or IL-29 polypeptide of the present invention that have retained some biological activity associated with IL-28A, IL-28B or IL-29, alone or in combination with other biologics or pharmaceuticals, to an immunocompromised mammal with a viral infection, thereby reducing the viral infection, such as is described above. All of the above methods of the present invention can also comprise the administration of zcyto24 or zcyto25 as well.

IL-28 and IL-29 are known to have an odd number of cysteines (PCT application WO 02/086087 and Sheppard et al., supra.) Expression of recombinant IL-28 and IL-29 can result in a heterogeneous mixture of proteins composed of intramolecular disulfide bonding in multiple conformations. The separation of these forms can be difficult and laborious. It is therefore desirable to provide IL-28 and IL-29 molecules having a single intramolecular disulfide bonding pattern upon expression and methods for refolding and purifying these preparations to maintain homogeneity. Thus, the present invention provides for compositions and methods to produce homogeneous preparations of IL-28 and IL-29.

The present invention provides polynucleotide molecules, including DNA and RNA molecules, that encode Cysteine mutants of IL-28 and IL-29 that result in expression of a recombinant IL-28 or IL-29 preparation that is a homogeneous preparation. For the purposes of this invention, a homogeneous preparation of IL-28 and IL-29 is a preparation in which comprises at least 98% of a single intramolecular disulfide bonding pattern in the purified polypeptide. In other embodiments, the single disulfide conformation in a preparation of purified polypeptide is at 99% homogeneous. In general, these Cysteine mutants will maintain some biological activity of the wildtype IL-28 or IL-29, as described herein. For example, the molecules of the present invention can bind to the IL-28 receptor with some specificity. Generally, a ligand binding to its cognate receptor is specific when the K_{D} falls within the range of 100 nM to 100 pM. Specific binding in the range of 100 mM to 10 nM K_{D} is low affinity binding. Specific binding in the range of 2.5 pM to 100 pM K_{D} is high affinity binding. In another example, biological activity of IL-28 or IL-29 Cysteine mutants is present when the molecules are capable of some level of antiviral activity associated with wildtype IL-28 or IL-29. Determination of the level of antiviral activity is described in detail herein.

An IL-28A gene encodes a polypeptide of 200 amino acids, as shown in SEQ ID NO:2. The signal sequence for IL-28A comprises amino acid residue 1 (Met) through amino acid residue 21 (Ala) of SEQ ID NO:2. The mature peptide for IL-28A begins at amino acid residue 22 (Val). A variant IL-28A gene encodes a polypeptide of 200 amino acids, as shown in SEQ ID NO:18. The signal sequence for IL-28A can be predicted as comprising amino acid residue −25 (Met) through amino acid residue −1 (Ala) of SEQ ID NO:18. The mature peptide for IL-28A begins at amino acid residue 1 (Val). IL-28A helices are predicted as follow: helix A is defined by amino acid residues 31 (Ala) to 45 (Leu); helix B by amino acid residues 58 (Thr) to 65 (Gln); helix C by amino acid residues 69 (Arg) to 86 (Ala); helix D by amino acid residues 95 (Val) to 114 (Ala); helix E by amino acid residues 126 (Thr) to 142 (Lys); and helix F by amino acid residues 148 (Cys) to 169 (Ala); as shown in SEQ ID NO:18. When a polynucleotide sequence encoding the mature polypeptide is expressed in a prokaryotic system, such as E. coli, a secretory signal sequence may not be required and an N-terminal Met may be present, resulting in expression of a polypeptide such as, for instance, as shown in SEQ ID NO:36.

IL-28A polypeptides of the present invention also include a mutation at the second cysteine, C2, of the mature polypeptide. For example, C2 from the N-terminus of the polypeptide of SEQ ID NO:18 is the cysteine at amino acid position 48 (position 49, additional N-terminal Met, if expressed in E. coli, see, for example, SEQ ID NO:36). This second cysteine (of which there are seven, like IL-28B) or C2 of IL-28A can be mutated, for example, to a serine, alanine, threonine, valine, or asparagine. IL-28A C2 mutant molecules of the present invention include, for example, polynucleotide molecules as shown in SEQ ID NOs:23 and 25, including DNA and RNA molecules, that encode IL-28A C2 mutant polypeptides as shown in SEQ ID NOs:24 and 26, respectively.

The present invention also includes biologically active mutants of IL-28A C2 cysteine mutants which provide, at least partially, an antiviral activity as provided here, e.g., anti-hepatitis C activity. The second cysteine or C2 from the N-terminus of IL-28A can mutated to any amino acid that does not form a disulfide bond with another cysteine, e.g., serine, alanine, threonine, valine or aspargine. The biologically active mutants of IL-28A C2 cysteine mutants of the present invention include N-, C-, and N- and C-terminal deletions of IL-28A, e.g., the polypeptide of SEQ ID NO:138 encoded by the polynucleotide of SEQ ID NO:137.

N-terminally modified biologically active mutants of IL-28A C2 mutants include, for example, amino acid residues 3-176 of SEQ ID NO:138 which is encoded by nucleotides 7-528 of SEQ ID NO:137; amino acid residues 4-176 of SEQ ID NO:138 which is encoded by nucleotides 10-528 of SEQ ID NO:137; amino acid residues 5-176 of SEQ ID NO:138 which is encoded by nucleotides 13-528 of SEQ ID NO:137; amino acid residues 6-176 of SEQ ID NO:138 which is encoded by nucleotides 16-528 of SEQ ID NO:137; amino acid residues 7-176 of SEQ ID NO:138 which is encoded by nucleotides 19-528 of SEQ ID NO:137; amino acid residues 8-176 of SEQ ID NO:138 which is encoded by nucleotides 22-528 of SEQ ID NO:137; amino acid residues 9-176 of SEQ ID NO:138 which is encoded by nucleotides 25-528 of SEQ ID NO:137; amino acid residues 10-176 of SEQ ID NO:138 which is encoded by nucleotides 28-528 of SEQ ID NO:137; amino acid residues 11-176 of SEQ ID NO:138 which is encoded by nucleotides 31-528 of SEQ ID NO:137; amino acid residues 12-176 of SEQ ID NO:138 which is encoded by nucleotides 34-528 of SEQ ID NO:137; amino acid residues 13-176 of SEQ ID NO:138 which is encoded by nucleotides 37-528 of SEQ ID NO:137; amino acid residues 14-176 of SEQ ID NO:138 which is encoded by nucleotides 40-528 of SEQ ID NO:137; amino acid residues 15-176 of SEQ ID NO:138 which is encoded by nucleotides 43-528 of SEQ ID NO:137; and amino acid residues 16-176 of SEQ ID NO:138 which is encoded by nucleotides 46-528 of SEQ ID NO:137. The N-terminally modified biologically active mutants of IL-28A C2 mutants of the present invention may also include an N-terminal Methione if expressed, for instance, in E. coli.

C-terminally modified biologically active mutants of IL-28A C2 mutants include, for example, amino acid residues 1-175 of SEQ ID NO:138 which is encoded by nucleotides 1-525 of SEQ ID NO:137.

N-terminally and C-terminally modified biologically active mutants of IL-28A C2 mutants include, for example, amino acid residues 2-175 of SEQ ID NO:138 which is encoded by nucleotides 4-525 of SEQ ID NO:137; amino acid residues 3-175 of SEQ ID NO:138 which is encoded by nucleotides 7-525 of SEQ ID NO:137; amino acid residues 4-175 of SEQ ID NO:138 which is encoded by nucleotides 10-525 of SEQ ID NO:137; amino acid residues 5-175 of SEQ ID NO:138 which is encoded by nucleotides 13-525 of SEQ ID NO:137; amino acid residues 6-175 of SEQ ID NO:138 which is encoded by nucleotides 16-525 of SEQ ID NO:137; amino acid residues 7-175 of SEQ ID NO:138 which is encoded by nucleotides 19-525 of SEQ ID NO:137; amino acid residues 8-175 of SEQ ID NO:138 which is encoded by nucleotides 22-525 of SEQ ID NO:137; amino acid residues 9-175 of SEQ ID NO:138 which is encoded by nucleotides 25-525 of SEQ ID NO:137; amino acid residues 10-175 of SEQ ID NO:138 which is encoded by nucleotides 28-525 of SEQ ID NO:137; amino acid residues 11-175 of SEQ ID NO:138 which is encoded by nucleotides 31-525 of SEQ ID NO:137; amino acid residues 12-175 of SEQ ID NO:138 which is encoded by nucleotides 34-525 of SEQ ID NO:137; amino acid residues 13-175 of SEQ ID NO:138 which is encoded by nucleotides 37-525 of SEQ ID NO:137; amino acid residues 14-175 of SEQ ID NO:138 which is encoded by nucleotides 40-525 of SEQ ID NO:137; amino acid residues 15-175 of SEQ ID NO:138 which is encoded by nucleotides 43-525 of SEQ ID NO:137; amino acid residues 16-175 of SEQ ID NO:138 which is encoded by nucleotides 46-525 of SEQ ID NO:137; and amino acid residues 17-175 of SEQ ID NO:138 which is encoded by nucleotides 49-525 of SEQ ID NO:137. The N-terminally and C-terminally modified biologically active mutants of IL-28A C2 mutants of the present invention may also include an N-terminal Methione if expressed, for instance, in E. coli.

In addition to the IL-28A C2 mutants, the present invention also includes IL-28A polypeptides comprising a mutation at the third cysteine position, C3, of the mature polypeptide. For example, C3 from the N-terminus of the polypeptide of SEQ ID NO:18, is the cysteine at position 50, (position 51, additional N-terminal Met, if expressed in E. coli, see, for example, SEQ ID NO:36). IL-28A C3 mutant molecules of the present invention include, for example, polynucleotide molecules as shown in SEQ ID NOs:27 and 29, including DNA and RNA molecules, that encode IL-28A C3 mutant polypeptides as shown in SEQ ID NOs:28 and 30, respectively (PCT publication WO 03/066002 (Kotenko et al.)).

The present invention also includes biologically active mutants of IL-28A C3 cysteine mutants which provide, at least partially, an antiviral activity as provided here, e.g., anti-hepatitis C activity. The third cysteine or C3 from the N-terminus of IL-28A can mutated to any amino acid that does not form a disulfide bond with another cysteine, e.g., serine, alanine, threonine, valine or aspargine. The biologically active mutants of IL-28A C3 cysteine mutants of the present invention include N-, C-, and N- and C-terminal deletions of IL-28A, e.g., the polypeptide of SEQ ID NO:140 encoded by the polynucleotide of SEQ ID NO:139.

N-terminally modified biologically active mutants of IL-28A C3 mutants include, for example, amino acid residues 2-176 of SEQ ID NO:140 which is encoded by nucleotides 4-528 of SEQ ID NO:139; amino acid residues 3-176 of SEQ ID NO:140 which is encoded by nucleotides 7-528 of SEQ ID NO:139; amino acid residues 4-176 of SEQ ID NO:140 which is encoded by nucleotides 10-528 of SEQ ID NO:139; amino acid residues 5-176 of SEQ ID NO:140 which is encoded by nucleotides 13-528 of SEQ ID NO:139; amino acid residues 6-176 of SEQ ID NO:140 which is encoded by nucleotides 16-528 of SEQ ID NO:139; amino acid residues 7-176 of SEQ ID NO:140 which is encoded by nucleotides 19-528 of SEQ ID NO:139; amino acid residues 8-176 of SEQ ID NO:140 which is encoded by nucleotides 22-528 of SEQ ID NO:139; amino acid residues 9-176 of SEQ ID NO:140 which is encoded by nucleotides 25-528 of SEQ ID NO:139; amino acid residues 10-176 of SEQ ID NO:140 which is encoded by nucleotides 28-528 of SEQ ID NO:139; amino acid residues 11-176 of SEQ ID NO:140 which is encoded by nucleotides 31-528 of SEQ ID NO:139; amino acid residues 12-176 of SEQ ID NO:140 which is encoded by nucleotides 34-528 of SEQ ID NO:139; amino acid residues 13-176 of SEQ ID NO:140 which is encoded by nucleotides 37-528 of SEQ ID NO:139; amino acid residues 14-176 of SEQ ID NO:140 which is encoded by nucleotides 40-528 of SEQ ID NO:139; amino acid residues 15-176 of SEQ ID NO:140 which is encoded by nucleotides 43-528 of SEQ ID NO:139; and amino acid residues 16-176 of SEQ ID NO:140 which is encoded by nucleotides 46-528 of SEQ ID NO:139. The N-terminally modified biologically active mutants of IL-28A C3 mutants of the present invention may also include an N-terminal Methione if expressed, for instance, in E. coli.

C-terminally modified biologically active mutants of IL-28A C3 mutants include, for example, amino acid residues 1-175 of SEQ ID NO:140 which is encoded by nucleotides 1-525 of SEQ ID NO:139.

N-terminally and C-terminally modified biologically active mutants of IL-28A C3 mutants include, for example, amino acid residues 2-175 of SEQ ID NO:140 which is encoded by nucleotides 4-525 of SEQ ID NO:139; amino acid residues 3-175 of SEQ ID NO:140 which is encoded by nucleotides 7-525 of SEQ ID NO:139; amino acid residues 4-175 which is encoded by nucleotides 10-525 of SEQ ID NO:139; amino acid residues 5-175 of SEQ ID NO:140 which is encoded by nucleotides 13-525 of SEQ ID NO:139; amino acid residues 6-175 of SEQ ID NO:140 which is encoded by nucleotides 16-525 of SEQ ID NO:139; amino acid residues 7-175 of SEQ ID NO:140 which is encoded by nucleotides 19-525 of SEQ ID NO:139; amino acid residues 8-175 of SEQ ID NO:140 which is encoded by nucleotides 22-525 of SEQ ID NO:139; amino acid residues 9-175 of SEQ ID NO:140 which is encoded by nucleotides 25-525 of SEQ ID NO:139; amino acid residues 10-175 of SEQ ID NO:140 which is encoded by nucleotides 28-525 of SEQ ID NO:139; amino acid residues 11-175 of SEQ ID NO:140 which is encoded by nucleotides 31-525 of SEQ ID NO:139; amino acid residues 12-175 of SEQ ID NO:140 which is encoded by nucleotides 34-525 of SEQ ID NO:139; amino acid residues 13-175 of SEQ ID NO:140 which is encoded by nucleotides 37-525 of SEQ ID NO:139; amino acid residues 14-175 of SEQ ID NO:140 which is encoded by nucleotides 40-525 of SEQ ID NO:139; amino acid residues 15-175 of SEQ ID NO:140 which is encoded by nucleotides 43-525 of SEQ ID NO:139; amino acid residues 16-175 of SEQ ID NO:140 which is encoded by nucleotides 46-525 of SEQ ID NO:139; and amino acid residues 17-175 of SEQ ID NO:140 which is encoded by nucleotides 49-525 of SEQ ID NO:139. The N-terminally and C-terminally modified biologically active mutants of IL-28A C3 mutants of the present invention may also include an N-terminal Methione if expressed, for instance, in E. coli.

The IL-28A polypeptides of the present invention include, for example, SEQ ID NOs:2, 18, 24, 26, 28, 30, 36, 138 and 140, and biologically active mutants, fusions, variants and fragments thereof which are encoded by IL-28A polynucleotide molecules as shown in SEQ ID NOs:1, 17, 23, 25, 27, 29, 35, 137 and 139, respectively.

An IL-29 gene encodes a polypeptide of 200 amino acids, as shown in SEQ ID NO:4. The signal sequence for IL-29 comprises amino acid residue 1 (Met) through amino acid residue 19 (Ala) of SEQ ID NO:4. The mature peptide for IL-29 begins at amino acid residue 20 (Gly). IL-29 has been described in published PCT application WO 02/02627. A variant IL-29 gene encodes a polypeptide of 200 amino acids, as shown in, for example, SEQ ID NO:20, where amino acid residue 188 (or amino acid residue 169 of the mature polypeptide which begins from amino acid residue 20 (Gly)) is Asn instead of Asp. The present invention also provides a variant IL-29 gene wherein the mature polypeptide has a Thr at amino acid residue 10 substituted with a Pro, such as, for instance, SEQ ID NOs:54, 56, 58, 60, 62, 64, 66, 68, 146, 148 and 150 which are encoded by the polynucleotide sequences as shown in SEQ ID NOs:53, 55, 57, 59, 61, 63, 65, 67, 145, 147 and 149, respectively. The present invention also provides a variant IL-29 gene wherein the mature polypeptide has a Gly at amino acid residue 18 substituted with an Asp, such as, for instance, SEQ ID NOs:70, 72, 74, 76, 78, 80, 82, 84, 146 and 148, which are encoded by the polynucleotide sequences as shown in SEQ ID NOs:69, 71, 73, 75, 77, 79, 81, 83, 145 and 147, respectively. The signal sequence for IL-29 can be predicted as comprising amino acid residue −19 (Met) through amino acid residue −1 (Ala) of SEQ ID NO:20. The mature peptide for IL-29 begins at amino acid residue 1 (Gly) of SEQ ID NO:20. IL-29 has been described in PCT application WO 02/02627. IL-29 helices are predicted as follows: helix A is defined by amino acid residues 30 (Ser) to 44 (Leu); helix B by amino acid residues 57 (Asn) to 65 (Val); helix C by amino acid residues 70 (Val) to 85 (Ala); helix D by amino acid residues 92 (Glu) to 114 (Gln); helix E by amino acid residues 118 (Thr) to 139 (Lys); and helix F by amino acid residues 144 (Gly) to 170 (Leu); as shown in SEQ ID NO:20. When a polynucleotide sequence encoding the mature polypeptide is expressed in a prokaryotic system, such as E. coli, a secretory signal sequence may not be required and an N-terminal Met may be present, resulting in expression of an IL-29 polypeptide such as, for instance, as shown in SEQ ID NO:38.

IL-29 polypeptides of the present invention also include a mutation at the fifth cysteine, C5, of the mature polypeptide. For example, C5 from the N-terminus of the polypeptide of SEQ ID NO:20, is the cysteine at position 171, or position 172 (additional N-terminal Met) if expressed in E. coli. (see, for example, SEQ ID NO:38). This fifth cysteine or C5 of IL-29 can be mutated, for example, to a serine, alanine, threonine, valine, or asparagine. These IL-29 C5 mutant polypeptides have a disulfide bond pattern of C1(Cys15 of SEQ ID NO:20)/C3(Cys112 of SEQ ID NO:20) and C2(Cys49 of SEQ ID NO:20)/C4(Cys145 of SEQ ID NO:20). IL-29 C5 mutant molecules of the present invention include, for example, polynucleotide molecules as shown in SEQ ID NOs:31, 33, 49, 51, 109, 111, 113, 115, 117, 119, 121, 123, 125, 127, 129, 131, 133, 135, 147 and 149, including DNA and RNA molecules, that encode IL-29 C5 mutant polypeptides as shown in SEQ ID NOs:32, 34, 50, 52, 110, 112, 114, 116, 118, 120, 122, 124, 126, 128, 130, 132, 134, 136, 148 and 150, respectively. Additional IL-29 C5 mutant molecules of the present invention include polynucleotide molecules as shown in SEQ ID NOs:53, 55, 61, and 63, including DNA and RNA molecules, that encode IL-29 C5 mutant polypeptides as shown in SEQ ID NOs:54, 55, 62, and 64, respectively (PCT publication WO 03/066002 (Kotenko et al.)). Additional, IL-29 C5 mutant molecules of the present invention include polynucleotide molecules as shown in SEQ ID NOs:69, 71, 77, and 79, including DNA and RNA molecules, that encode IL-29 C5 mutant polypeptides as shown in SEQ ID NOs:70, 72, 78, and 80, respectively (PCT publication WO 02/092762 (Baum et al.)).

The present invention also includes biologically active mutants of IL-29 C5 cysteine mutants which provide, at least partially, an antiviral activity as provided here, e.g., anti-hepatitis C activity. The fifth cysteine or C5 from the N-terminus of IL-29 can mutated to any amino acid that does not form a disulfide bond with another cysteine, e.g., serine, alanine, threonine, valine or aspargine. The biologically active mutants of IL-29 C5 cysteine mutants of the present invention include N-, C-, and N- and C-terminal deletions of IL-29, e.g., the polypeptides of SEQ ID NOs:148 and 150 encoded by the polynucleotides of SEQ ID NOs:147 and 149, respectively.

N-terminally modified biologically active mutants of IL-29 C5 mutants include, for example, amino acid residues 2-182 of SEQ ID NO:148 which is encoded by nucleotides 4-546 of SEQ ID NO:147; amino acid residues 3-182 of SEQ ID NO:148 which is encoded by nucleotides 7-546 of SEQ ID NO:147; amino acid residues 4-182 of SEQ ID NO:148 which is encoded by nucleotides 10-546 of SEQ ID NO:147; amino acid residues 5-182 of SEQ ID NO:148 which is encoded by nucleotides 13-546 of SEQ ID NO:147; amino acid residues 6-182 of SEQ ID NO:148 which is encoded by nucleotides 16-546 of SEQ ID NO:147; amino acid residues 7-182 of SEQ ID NO:148 which is encoded by nucleotides 19-546 of SEQ ID NO:147; amino acid residues 8-182 of SEQ ID NO:148 which is encoded by nucleotides 22-546 of SEQ ID NO:147; amino acid residues 9-182 of SEQ ID NO:148 which is encoded by nucleotides 25-546 of SEQ ID NO:147; amino acid residues 10-182 of SEQ ID NO:148 which is encoded by nucleotides 28-546 of SEQ ID NO:147; amino acid residues 11-182 of SEQ ID NO:148 which is encoded by nucleotides 31-546 of SEQ ID NO:147; amino acid residues 12-182 of SEQ ID NO:148 which is encoded by nucleotides 34-546 of SEQ ID NO:147; amino acid residues 13-182 of SEQ ID NO:148 which is encoded by nucleotides 37-546 of SEQ ID NO:147; amino acid residues 14-182 of SEQ ID NO:148 which is encoded by nucleotides 40-546 of SEQ ID NO:147; amino acid residues 15-182 of SEQ ID NO:148 which is encoded by nucleotides 43-546 of SEQ ID NO:147; amino acid residues 2-176 of SEQ ID NO:150 which is encoded by nucleotides 4-528 of SEQ ID NO:149; amino acid residues 3-176 of SEQ ID NO:150 which is encoded by nucleotides 7-528 of SEQ ID NO:149; amino acid residues 4-176 of SEQ ID NO:150 which is encoded by nucleotides 10-528 of SEQ ID NO:149; amino acid residues 5-176 of SEQ ID NO:150 which is encoded by nucleotides 13-528 of SEQ ID NO:149; amino acid residues 6-176 of SEQ ID NO:150 which is encoded by nucleotides 16-528 of SEQ ID NO:149; amino acid residues 7-176 of SEQ ID NO:150 which is encoded by nucleotides 19-528 of SEQ ID NO:149; amino acid residues 8-176 of SEQ ID NO:150 which is encoded by nucleotides 22-528 of SEQ ID NO:149; and amino acid residues 9-176 of SEQ ID NO:150 which is encoded by nucleotides 25-528 of SEQ ID NO:149. The N-terminally modified biologically active mutants of IL-29 C5 mutants of the present invention may also include an N-terminal Methione if expressed, for instance, in E. coli.

C-terminally modified biologically active mutants of IL-29 C5 mutants include, for example, amino acid residues 1-181 of SEQ ID NO:148 which is encoded by nucleotides 1-543 of SEQ ID NO:147; amino acid residues 1-180 of SEQ ID NO:148 which is encoded by nucleotides 1-540 of SEQ ID NO:147; amino acid residues 1-179 of SEQ ID NO:148 which is encoded by nucleotides 1-537 of SEQ ID NO:147; amino acid residues 1-178 of SEQ ID NO:148 which is encoded by nucleotides 1-534 of SEQ ID NO:147; amino acid residues 1-177 of SEQ ID NO:148 which is encoded by nucleotides 1-531 of SEQ ID NO:147; amino acid residues 1-176 of SEQ ID NO:148 which is encoded by nucleotides 1-528 of SEQ ID NO:147; amino acid residues 1-175 of SEQ ID NO:148 which is encoded by nucleotides 1-525 of SEQ ID NO:147; amino acid residues 1-174 of SEQ ID NO:148 which is encoded by nucleotides 1-522 of SEQ ID NO:147; amino acid residues 1-173 of SEQ ID NO:148 which is encoded by nucleotides 1-519 of SEQ ID NO:147; amino acid residues 1-172 of SEQ ID NO:148 which is encoded by nucleotides 1-516 of SEQ ID NO:147; amino acid residues 1-175 of SEQ ID NO:150 which is encoded by nucleotides 1-525 of SEQ ID NO:149; amino acid residues 1-174 of SEQ ID NO:150 which is encoded by nucleotides 1-522 of SEQ ID NO:149; amino acid residues 1-173 of SEQ ID NO:150 which is encoded by nucleotides 1-519 of SEQ ID NO:149; amino acid residues 1-172 of SEQ ID NO:150 which is encoded by nucleotides 1-516 of SEQ ID NO:149; amino acid residues 1-171 of SEQ ID NO:150 which is encoded by nucleotides 1-513 of SEQ ID NO:149; amino acid residues 1-170 of SEQ ID NO:150 which is encoded by nucleotides 1-510 of SEQ ID NO:149; amino acid residues 1-169 of SEQ ID NO:150 which is encoded by nucleotides 1-507 of SEQ ID NO:149; amino acid residues 1-168 of SEQ ID NO:150 which is encoded by nucleotides 1-504 of SEQ ID NO:149; amino acid residues 1-167 of SEQ ID NO:150 which is encoded by nucleotides 1-501 of SEQ ID NO:149; and amino acid residues 1-166 of SEQ ID NO:150 which is encoded by nucleotides 1-498 of SEQ ID NO:149.

N-terminally and C-terminally modified biologically active mutants of IL-29 C5 mutants include, for example, amino acid residues 2-182 of SEQ ID NO:148 which is encoded by nucleotides 4-546 of SEQ ID NO:147; amino acid residues 2-181 of SEQ ID NO:148 which is encoded by nucleotides 4-543 of SEQ ID NO:147; amino acid residues 2-180 of SEQ ID NO:148 which is encoded by nucleotides 4-540 of SEQ ID NO:147; amino acid residues 2-179 of SEQ ID NO:148 which is encoded by nucleotides 4-537 of SEQ ID NO:147; amino acid residues 2-178 of SEQ ID NO:148 which is encoded by nucleotides 4-534 of SEQ ID NO:147; amino acid residues 2-177 of SEQ ID NO:148 which is encoded by nucleotides 4-531 of SEQ ID NO:147; amino acid residues 2-176 of SEQ ID NO:148 which is encoded by nucleotides 4-528 of SEQ ID NO:147; amino acid residues 2-175 of SEQ ID NO:148 which is encoded by nucleotides 4-525 of SEQ ID NO:147; amino acid residues 2-174 of SEQ ID NO:148 which is encoded by nucleotides 4-522 of SEQ ID NO:147; amino acid residues 2-173 of SEQ ID NO:148 which is encoded by nucleotides 4-519 of SEQ ID NO:147; amino acid residues 2-172 of SEQ ID NO:148 which is encoded by nucleotides 4-516 of SEQ ID NO:147; amino acid residues 3-182 of SEQ ID NO:148 which is encoded by nucleotides 7-546 of SEQ ID NO:147; amino acid residues 3-181 of SEQ ID NO:148 which is encoded by nucleotides 7-543 of SEQ ID NO:147; amino acid residues 3-180 of SEQ ID NO:148 which is encoded by nucleotides 7-540 of SEQ ID NO:147; amino acid residues 3-179 of SEQ ID NO:148 which is encoded by nucleotides 7-537 of SEQ ID NO:147; amino acid residues 3-178 of SEQ ID NO:148 which is encoded by nucleotides 7-534 of SEQ ID NO:147; amino acid residues 3-177 of SEQ ID NO:148 which is encoded by nucleotides 7-531 of SEQ ID NO:147; amino acid residues 3-176 of SEQ ID NO:148 which is encoded by nucleotides 7-528 of SEQ ID NO:147; amino acid residues 3-175 of SEQ ID NO:148 which is encoded by nucleotides 7-525 of SEQ ID NO:147; amino acid residues 3-174 of SEQ ID NO:148 which is encoded by nucleotides 7-522 of SEQ ID NO:147; amino acid residues 3-173 of SEQ ID NO:148 which is encoded by nucleotides 7-519 of SEQ ID NO:147; amino acid residues 3-172 of SEQ ID NO:148 which is encoded by nucleotides 7-516 of SEQ ID NO:147; amino acid residues 4-182 of SEQ ID NO:148 which is encoded by nucleotides 10-546 of SEQ ID NO:147; amino acid residues 4-181 of SEQ ID NO:148 which is encoded by nucleotides 10-543 of SEQ ID NO:147; amino acid residues 4-180 of SEQ ID NO:148 which is encoded by nucleotides 10-540 of SEQ ID NO:147; amino acid residues 4-179 of SEQ ID NO:148 which is encoded by nucleotides 10-537 of SEQ ID NO:147; amino acid residues 4-178 of SEQ ID NO:148 which is encoded by nucleotides 10-534 of SEQ ID NO:147; amino acid residues 4-177 of SEQ ID NO:148 which is encoded by nucleotides 10-531 of SEQ ID NO:147; amino acid residues 4-176 of SEQ ID NO:148 which is encoded by nucleotides 10-528 of SEQ ID NO:147; amino acid residues 4-175 of SEQ ID NO:148 which is encoded by nucleotides 10-525 of SEQ ID NO:147; amino acid residues 4-174 of SEQ ID NO:148 which is encoded by nucleotides 10-522 of SEQ ID NO:147; amino acid residues 4-173 of SEQ ID NO:148 which is encoded by nucleotides 10-519 of SEQ ID NO:147; amino acid residues 4-172 of SEQ ID NO:148 which is encoded by nucleotides 10-516 of SEQ ID NO:147; amino acid residues 5-182 of SEQ ID NO:148 which is encoded by nucleotides 13-546 of SEQ ID NO:147; amino acid residues 5-181 of SEQ ID NO:148 which is encoded by nucleotides 13-543 of SEQ ID NO:147; amino acid residues 5-180 of SEQ ID NO:148 which is encoded by nucleotides 13-540 of SEQ ID NO:147; amino acid residues 5-179 of SEQ ID NO:148 which is encoded by nucleotides 13-537 of SEQ ID NO:147; amino acid residues 5-178 of SEQ ID NO:148 which is encoded by nucleotides 13-534 of SEQ ID NO:147; amino acid residues 5-177 of SEQ ID NO:148 which is encoded by nucleotides 13-531 of SEQ ID NO:147; amino acid residues 5-176 of SEQ ID NO:148 which is encoded by nucleotides 13-528 of SEQ ID NO:147; amino acid residues 5-175 of SEQ ID NO:148 which is encoded by nucleotides 13-525 of SEQ ID NO:147; amino acid residues 5-174 of SEQ ID NO:148 which is encoded by nucleotides 13-522 of SEQ ID NO:147; amino acid residues 5-173 of SEQ ID NO:148 which is encoded by nucleotides 13-519 of SEQ ID NO:147; amino acid residues 5-172 of SEQ ID NO:148 which is encoded by nucleotides 13-516 of SEQ ID NO:147; amino acid residues 6-182 of SEQ ID NO:148 which is encoded by nucleotides 16-546 of SEQ ID NO:147; amino acid residues 6-181 of SEQ ID NO:148 which is encoded by nucleotides 16-543 of SEQ ID NO:147; amino acid residues 6-180 of SEQ ID NO:148 which is encoded by nucleotides 16-540 of SEQ ID NO:147; amino acid residues 6-179 of SEQ ID NO:148 which is encoded by nucleotides 16-537 of SEQ ID NO:147; amino acid residues 6-178 of SEQ ID NO:148 which is encoded by nucleotides 16-534 of SEQ ID NO:147; amino acid residues 6-177 of SEQ ID NO:148 which is encoded by nucleotides 16-531 of SEQ ID NO:147; amino acid residues 6-176 of SEQ ID NO:148 which is encoded by nucleotides 16-528 of SEQ ID NO:147; amino acid residues 6-175 of SEQ ID NO:148 which is encoded by nucleotides 16-525 of SEQ ID NO:147; amino acid residues 6-174 of SEQ ID NO:148 which is encoded by nucleotides 16-522 of SEQ ID NO:147; amino acid residues 6-173 of SEQ ID NO:148 which is encoded by nucleotides 16-519 of SEQ ID NO:147; amino acid residues 6-172 of SEQ ID NO:148 which is encoded by nucleotides 16-516 of SEQ ID NO:147; amino acid residues 7-182 of SEQ ID NO:148 which is encoded by nucleotides 19-546 of SEQ ID NO:147; amino acid residues 7-181 of SEQ ID NO:148 which is encoded by nucleotides 19-543 of SEQ ID NO:147; amino acid residues 7-180 of SEQ ID NO:148 which is encoded by nucleotides 19-540 of SEQ ID NO:147; amino acid residues 7-179 of SEQ ID NO:148 which is encoded by nucleotides 19-537 of SEQ ID NO:147; amino acid residues 7-178 of SEQ ID NO:148 which is encoded by nucleotides 19-534 of SEQ ID NO:147; amino acid residues 7-177 of SEQ ID NO:148 which is encoded by nucleotides 19-531 of SEQ ID NO:147; amino acid residues 7-176 of SEQ ID NO:148 which is encoded by nucleotides 19-528 of SEQ ID NO:147; amino acid residues 7-175 of SEQ ID NO:148 which is encoded by nucleotides 19-525 of SEQ ID NO:147; amino acid residues 7-174 of SEQ ID NO:148 which is encoded by nucleotides 19-522 of SEQ ID NO:147; amino acid residues 7-173 of SEQ ID NO:148 which is encoded by nucleotides 19-519 of SEQ ID NO:147; amino acid residues 7-172 of SEQ ID NO:148 which is encoded by nucleotides 19-516 of SEQ ID NO:147; amino acid residues 8-182 of SEQ ID NO:148 which is encoded by nucleotides 22-546 of SEQ ID NO:147; amino acid residues 8-181 of SEQ ID NO:148 which is encoded by nucleotides 22-543 of SEQ ID NO:147; amino acid residues 8-180 of SEQ ID NO:148 which is encoded by nucleotides 22-540 of SEQ ID NO:147; amino acid residues 8-179 of SEQ ID NO:148 which is encoded by nucleotides 22-537 of SEQ ID NO:147; amino acid residues 8-178 of SEQ ID NO:148 which is encoded by nucleotides 22-534 of SEQ ID NO:147; amino acid residues 8-177 of SEQ ID NO:148 which is encoded by nucleotides 22-531 of SEQ ID NO:147; amino acid residues 8-176 of SEQ ID NO:148 which is encoded by nucleotides 22-528 of SEQ ID NO:147; amino acid residues 8-175 of SEQ ID NO:148 which is encoded by nucleotides 22-525 of SEQ ID NO:147; amino acid residues 8-174 of SEQ ID NO:148 which is encoded by nucleotides 22-522 of SEQ ID NO:147; amino acid residues 8-173 of SEQ ID NO:148 which is encoded by nucleotides 22-519 of SEQ ID NO:147; amino acid residues 8-172 of SEQ ID NO:148 which is encoded by nucleotides 22-516 of SEQ ID NO:147; amino acid residues 9-182 of SEQ ID NO:148 which is encoded by nucleotides 25-546 of SEQ ID NO:147; amino acid residues 9-181 of SEQ ID NO:148 which is encoded by nucleotides 25-543 of SEQ ID NO:147; amino acid residues 9-180 of SEQ ID NO:148 which is encoded by nucleotides 25-540 of SEQ ID NO:147; amino acid residues 9-179 of SEQ ID NO:148 which is encoded by nucleotides 25-537 of SEQ ID NO:147; amino acid residues 9-178 of SEQ ID NO:148 which is encoded by nucleotides 25-534 of SEQ ID NO:147; amino acid residues 9-177 of SEQ ID NO:148 which is encoded by nucleotides 25-531 of SEQ ID NO:147; amino acid residues 9-176 of SEQ ID NO:148 which is encoded by nucleotides 25-528 of SEQ ID NO:147; amino acid residues 9-175 of SEQ ID NO:148 which is encoded by nucleotides 25-525 of SEQ ID NO:147; amino acid residues 9-174 of SEQ ID NO:148 which is encoded by nucleotides 25-522 of SEQ ID NO:147; amino acid residues 9-173 of SEQ ID NO:148 which is encoded by nucleotides 25-519 of SEQ ID NO:147; amino acid residues 9-172 of SEQ ID NO:148 which is encoded by nucleotides 25-516 of SEQ ID NO:147; amino acid residues 10-182 of SEQ ID NO:148 which is encoded by nucleotides 28-546 of SEQ ID NO:147; amino acid residues 10-181 of SEQ ID NO:148 which is encoded by nucleotides 28-543 of SEQ ID NO:147; amino acid residues 10-180 of SEQ ID NO:148 which is encoded by nucleotides 28-540 of SEQ ID NO:147; amino acid residues 10-179 of SEQ ID NO:148 which is encoded by nucleotides 28-537 of SEQ ID NO:147; amino acid residues 10-178 of SEQ ID NO:148 which is encoded by nucleotides 28-534 of SEQ ID NO:147; amino acid residues 10-177 of SEQ ID NO:148 which is encoded by nucleotides 28-531 of SEQ ID NO:147; amino acid residues 10-176 of SEQ ID NO:148 which is encoded by nucleotides 28-528 of SEQ ID NO:147; amino acid residues 10-175 of SEQ ID NO:148 which is encoded by nucleotides 28-525 of SEQ ID NO:147; amino acid residues 10-174 of SEQ ID NO:148 which is encoded by nucleotides 28-522 of SEQ ID NO:147; amino acid residues 10-173 of SEQ ID NO:148 which is encoded by nucleotides 28-519 of SEQ ID NO:147; amino acid residues 10-172 of SEQ ID NO:148 which is encoded by nucleotides 28-516 of SEQ ID NO:147; amino acid residues 11-182 of SEQ ID NO:148 which is encoded by nucleotides 31-546 of SEQ ID NO:147; amino acid residues 11-181 of SEQ ID NO:148 which is encoded by nucleotides 31-543 of SEQ ID NO:147; amino acid residues 11-180 of SEQ ID NO:148 which is encoded by nucleotides 31-540 of SEQ ID NO:147; amino acid residues 11-179 of SEQ ID NO:148 which is encoded by nucleotides 31-537 of SEQ ID NO:147; amino acid residues 11-178 of SEQ ID NO:148 which is encoded by nucleotides 31-534 of SEQ ID NO:147; amino acid residues 11-177 of SEQ ID NO:148 which is encoded by nucleotides 31-531 of SEQ ID NO:147; amino acid residues 11-176 of SEQ ID NO:148 which is encoded by nucleotides 31-528 of SEQ ID NO:147; amino acid residues 11-175 of SEQ ID NO:148 which is encoded by nucleotides 31-525 of SEQ ID NO:147; amino acid residues 11-174 of SEQ ID NO:148 which is encoded by nucleotides 31-522 of SEQ ID NO:147; amino acid residues 11-173 of SEQ ID NO:148 which is encoded by nucleotides 31-519 of SEQ ID NO:147; amino acid residues 11-172 of SEQ ID NO:148 which is encoded by nucleotides 31-516 of SEQ ID NO:147; amino acid residues 12-182 of SEQ ID NO:148 which is encoded by nucleotides 34-546 of SEQ ID NO:147; amino acid residues 12-181 of SEQ ID NO:148 which is encoded by nucleotides 34-543 of SEQ ID NO:147; amino acid residues 12-180 of SEQ ID NO:148 which is encoded by nucleotides 34-540 of SEQ ID NO:147; amino acid residues 12-179 of SEQ ID NO:148 which is encoded by nucleotides 34-537 of SEQ ID NO:147; amino acid residues 12-178 of SEQ ID NO:148 which is encoded by nucleotides 34-534 of SEQ ID NO:147; amino acid residues 12-177 of SEQ ID NO:148 which is encoded by nucleotides 34-531 of SEQ ID NO:147; amino acid residues 12-176 of SEQ ID NO:148 which is encoded by nucleotides 34-528 of SEQ ID NO:147; amino acid residues 12-175 of SEQ ID NO:148 which is encoded by nucleotides 34-525 of SEQ ID NO:147; amino acid residues 12-174 of SEQ ID NO:148 which is encoded by nucleotides 34-522 of SEQ ID NO:147; amino acid residues 12-173 of SEQ ID NO:148 which is encoded by nucleotides 34-519 of SEQ ID NO:147; amino acid residues 12-172 of SEQ ID NO:148 which is encoded by nucleotides 34-516 of SEQ ID NO:147; amino acid residues 13-182 of SEQ ID NO:148 which is encoded by nucleotides 37-546 of SEQ ID NO:147; amino acid residues 13-181 of SEQ ID NO:148 which is encoded by nucleotides 37-543 of SEQ ID NO:147; amino acid residues 13-180 of SEQ ID NO:148 which is encoded by nucleotides 37-540 of SEQ ID NO:147; amino acid residues 13-179 of SEQ ID NO:148 which is encoded by nucleotides 37-537 of SEQ ID NO:147; amino acid residues 13-178 of SEQ ID NO:148 which is encoded by nucleotides 37-534 of SEQ ID NO:147; amino acid residues 13-177 of SEQ ID NO:148 which is encoded by nucleotides 37-531 of SEQ ID NO:147; amino acid residues 13-176 of SEQ ID NO:148 which is encoded by nucleotides 37-528 of SEQ ID NO:147; amino acid residues 13-175 of SEQ ID NO:148 which is encoded by nucleotides 37-525 of SEQ ID NO:147; amino acid residues 13-174 of SEQ ID NO:148 which is encoded by nucleotides 37-522 of SEQ ID NO:147; amino acid residues 13-173 of SEQ ID NO:148 which is encoded by nucleotides 37-519 of SEQ ID NO:147; amino acid residues 13-172 of SEQ ID NO:148 which is encoded by nucleotides 37-516 of SEQ ID NO:147; amino acid residues 14-182 of SEQ ID NO:148 which is encoded by nucleotides 40-546 of SEQ ID NO:147; amino acid residues 14-181 of SEQ ID NO:148 which is encoded by nucleotides 40-543 of SEQ ID NO:147; amino acid residues 14-180 of SEQ ID NO:148 which is encoded by nucleotides 40-540 of SEQ ID NO:147; amino acid residues 14-179 of SEQ ID NO:148 which is encoded by nucleotides 40-537 of SEQ ID NO:147; amino acid residues 14-178 of SEQ ID NO:148 which is encoded by nucleotides 40-534 of SEQ ID NO:147; amino acid residues 14-177 of SEQ ID NO:148 which is encoded by nucleotides 40-531 of SEQ ID NO:147; amino acid residues 14-176 of SEQ ID NO:148 which is encoded by nucleotides 40-528 of SEQ ID NO:147; amino acid residues 14-175 of SEQ ID NO:148 which is encoded by nucleotides 40-525 of SEQ ID NO:147; amino acid residues 14-174 of SEQ ID NO:148 which is encoded by nucleotides 40-522 of SEQ ID NO:147; amino acid residues 40-173 of SEQ ID NO:148 which is encoded by nucleotides 40-519 of SEQ ID NO:147; amino acid residues 14-172 of SEQ ID NO:148 which is encoded by nucleotides 40-516 of SEQ ID NO:147; amino acid residues 15-182 of SEQ ID NO:148 which is encoded by nucleotides 43-546 of SEQ ID NO:147; amino acid residues 15-181 of SEQ ID NO:148 which is encoded by nucleotides 43-543 of SEQ ID NO:147; amino acid residues 15-180 of SEQ ID NO:148 which is encoded by nucleotides 43-540 of SEQ ID NO:147; amino acid residues 15-179 of SEQ ID NO:148 which is encoded by nucleotides 43-537 of SEQ ID NO:147; amino acid residues 15-178 of SEQ ID NO:148 which is encoded by nucleotides 43-534 of SEQ ID NO:147; amino acid residues 15-177 of SEQ ID NO:148 which is encoded by nucleotides 43-531 of SEQ ID NO:147; amino acid residues 15-176 of SEQ ID NO:148 which is encoded by nucleotides 43-528 of SEQ ID NO:147; amino acid residues 15-175 of SEQ ID NO:148 which is encoded by nucleotides 43-525 of SEQ ID NO:147; amino acid residues 15-174 of SEQ ID NO:148 which is encoded by nucleotides 43-522 of SEQ ID NO:147; amino acid residues 15-173 of SEQ ID NO:148 which is encoded by nucleotides 43-519 of SEQ ID NO:147; amino acid residues 15-172 of SEQ ID NO:148 which is encoded by nucleotides 43-516 of SEQ ID NO:147; amino acid residues 16-182 of SEQ ID NO:148 which is encoded by nucleotides 46-546 of SEQ ID NO:147; amino acid residues 16-181 of SEQ ID NO:148 which is encoded by nucleotides 46-543 of SEQ ID NO:147; amino acid residues 16-180 of SEQ ID NO:148 which is encoded by nucleotides 46-540 of SEQ ID NO:147; amino acid residues 16-179 of SEQ ID NO:148 which is encoded by nucleotides 46-537 of SEQ ID NO:147; amino acid residues 16-178 of SEQ ID NO:148 which is encoded by nucleotides 46-534 of SEQ ID NO:147; amino acid residues 16-177 of SEQ ID NO:148 which is encoded by nucleotides 46-531 of SEQ ID NO:147; amino acid residues 16-176 of SEQ ID NO:148 which is encoded by nucleotides 46-528 of SEQ ID NO:147; amino acid residues 16-175 of SEQ ID NO:148 which is encoded by nucleotides 46-525 of SEQ ID NO:147; amino acid residues 16-174 of SEQ ID NO:148 which is encoded by nucleotides 46-522 of SEQ ID NO:147; amino acid residues 16-173 of SEQ ID NO:148 which is encoded by nucleotides 46-519 of SEQ ID NO:147; and amino acid residues 16-172 of SEQ ID NO:148 which is encoded by nucleotides 46-516 of SEQ ID NO:147. The N-terminally and C-terminally modified biologically active mutants of IL-29 C5 mutants of the present invention may also include an N-terminal Methione if expressed, for instance, in E. coli.

Additional IL-29 C5 N-terminally and C-terminally biologically active mutants include, for example, amino acid residues 2-176 of SEQ ID NO:150 which is encoded by nucleotides 4-528 of SEQ ID NO:149; amino acid residues 2-175 of SEQ ID NO:150 which is encoded by nucleotides 4-525 of SEQ ID NO:149; amino acid residues 2-174 of SEQ ID NO:150 which is encoded by nucleotides 4-522 of SEQ ID NO:149; amino acid residues 2-173 of SEQ ID NO:150 which is encoded by nucleotides 4-519 of SEQ ID NO:149; amino acid residues 2-172 of SEQ ID NO:150 which is encoded by nucleotides 4-516 of SEQ ID NO:149; amino acid residues 2-171 of SEQ ID NO:150 which is encoded by nucleotides 4-513 of SEQ ID NO:149; amino acid residues 2-170 of SEQ ID NO:150 which is encoded by nucleotides 4-510 of SEQ ID NO:149; amino acid residues 2-169 of SEQ ID NO:150 which is encoded by nucleotides 4-507 of SEQ ID NO:149; amino acid residues 2-168 of SEQ ID NO:150 which is encoded by nucleotides 4-504 of SEQ ID NO:149; amino acid residues 2-167 of SEQ ID NO:150 which is encoded by nucleotides 4-501 of SEQ ID NO:149; amino acid residues 2-166 of SEQ ID NO:150 which is encoded by nucleotides 4-498 of SEQ ID NO:149; amino acid residues 3-176 of SEQ ID NO:150 which is encoded by nucleotides 7-528 of SEQ ID NO:149; amino acid residues 3-175 of SEQ ID NO:150 which is encoded by nucleotides 7-525 of SEQ ID NO:149; amino acid residues 3-174 of SEQ ID NO:150 which is encoded by nucleotides 7-522 of SEQ ID NO:149; amino acid residues 3-173 of SEQ ID NO:150 which is encoded by nucleotides 7-519 of SEQ ID NO:149; amino acid residues 3-172 of SEQ ID NO:150 which is encoded by nucleotides 7-516 of SEQ ID NO:149; amino acid residues 3-171 of SEQ ID NO:150 which is encoded by nucleotides 7-513 of SEQ ID NO:149; amino acid residues 3-170 of SEQ ID NO:150 which is encoded by nucleotides 7-510 of SEQ ID NO:149; amino acid residues 3-169 of SEQ ID NO:150 which is encoded by nucleotides 7-507 of SEQ ID NO:149; amino acid residues 3-168 of SEQ ID NO:150 which is encoded by nucleotides 7-504 of SEQ ID NO:149; amino acid residues 3-167 of SEQ ID NO:150 which is encoded by nucleotides 7-501 of SEQ ID NO:149; amino acid residues 3-166 of SEQ ID NO:150 which is encoded by nucleotides 7-498 of SEQ ID NO:149; amino acid residues 4-176 of SEQ ID NO:150 which is encoded by nucleotides 10-528 of SEQ ID NO:149; amino acid residues 4-175 of SEQ ID NO:150 which is encoded by nucleotides 10-525 of SEQ ID NO:149; amino acid residues 4-174 of SEQ ID NO:150 which is encoded by nucleotides 10-522 of SEQ ID NO:149; amino acid residues 4-173 of SEQ ID NO:150 which is encoded by nucleotides 10-519 of SEQ ID NO:149; amino acid residues 4-172 of SEQ ID NO:150 which is encoded by nucleotides 10-516 of SEQ ID NO:149; amino acid residues 4-171 of SEQ ID NO:150 which is encoded by nucleotides 10-513 of SEQ ID NO:149; amino acid residues 4-170 of SEQ ID NO:150 which is encoded by nucleotides 10-510 of SEQ ID NO:149; amino acid residues 4-169 of SEQ ID NO:150 which is encoded by nucleotides 10-507 of SEQ ID NO:149; amino acid residues 4-168 of SEQ ID NO:150 which is encoded by nucleotides 10-504 of SEQ ID NO:149; amino acid residues 4-167 of SEQ ID NO:150 which is encoded by nucleotides 10-501 of SEQ ID NO:149; amino acid residues 4-166 of SEQ ID NO:150 which is encoded by nucleotides 10-498 of SEQ ID NO:149; amino acid residues 5-176 of SEQ ID NO:150 which is encoded by nucleotides 13-528 of SEQ ID NO:149; amino acid residues 5-175 of SEQ ID NO:150 which is encoded by nucleotides 13-525 of SEQ ID NO:149; amino acid residues 5-174 of SEQ ID NO:150 which is encoded by nucleotides 13-522 of SEQ ID NO:149; amino acid residues 5-173 of SEQ ID NO:150 which is encoded by nucleotides 13-519 of SEQ ID NO:149; amino acid residues 5-172 of SEQ ID NO:150 which is encoded by nucleotides 13-516 of SEQ ID NO:149; amino acid residues 5-171 of SEQ ID NO:150 which is encoded by nucleotides 13-513 of SEQ ID NO:149; amino acid residues 5-170 of SEQ ID NO:150 which is encoded by nucleotides 13-510 of SEQ ID NO:149; amino acid residues 5-169 of SEQ ID NO:150 which is encoded by nucleotides 13-507 of SEQ ID NO:149; amino acid residues 5-168 of SEQ ID NO:150 which is encoded by nucleotides 13-504 of SEQ ID NO:149; amino acid residues 5-167 of SEQ ID NO:150 which is encoded by nucleotides 13-501 of SEQ ID NO:149; amino acid residues 5-166 of SEQ ID NO:150 which is encoded by nucleotides 13-498 of SEQ ID NO:149; amino acid residues 6-176 of SEQ ID NO:150 which is encoded by nucleotides 16-528 of SEQ ID NO:149; amino acid residues 6-175 of SEQ ID NO:150 which is encoded by nucleotides 16-525 of SEQ ID NO:149; amino acid residues 6-174 of SEQ ID NO:150 which is encoded by nucleotides 16-522 of SEQ ID NO:149; amino acid residues 6-173 of SEQ ID NO:150 which is encoded by nucleotides 16-519 of SEQ ID NO:149; amino acid residues 6-172 of SEQ ID NO:150 which is encoded by nucleotides 16-516 of SEQ ID NO:149; amino acid residues 6-171 of SEQ ID NO:150 which is encoded by nucleotides 16-513 of SEQ ID NO:149; amino acid residues 6-170 of SEQ ID NO:150 which is encoded by nucleotides 16-510 of SEQ ID NO:149; amino acid residues 6-169 of SEQ ID NO:150 which is encoded by nucleotides 16-507 of SEQ ID NO:149; amino acid residues 6-168 of SEQ ID NO:150 which is encoded by nucleotides 16-504 of SEQ ID NO:149; amino acid residues 6-167 of SEQ ID NO:150 which is encoded by nucleotides 16-501 of SEQ ID NO:149; amino acid residues 6-166 of SEQ ID NO:150 which is encoded by nucleotides 16-498 of SEQ ID NO:149; amino acid residues 7-176 of SEQ ID NO:150 which is encoded by nucleotides 19-528 of SEQ ID NO:149; amino acid residues 7-175 of SEQ ID NO:150 which is encoded by nucleotides 19-525 of SEQ ID NO:149; amino acid residues 7-174 of SEQ ID NO:150 which is encoded by nucleotides 19-522 of SEQ ID NO:149; amino acid residues 7-173 of SEQ ID NO:150 which is encoded by nucleotides 19-519 of SEQ ID NO:149; amino acid residues 7-172 of SEQ ID NO:150 which is encoded by nucleotides 19-516 of SEQ ID NO:149; amino acid residues 7-171 of SEQ ID NO:150 which is encoded by nucleotides 19-513 of SEQ ID NO:149; amino acid residues 7-170 of SEQ ID NO:150 which is encoded by nucleotides 19-510 of SEQ ID NO:149; amino acid residues 7-169 of SEQ ID NO:150 which is encoded by nucleotides 19-507 of SEQ ID NO:149; amino acid residues 7-168 of SEQ ID NO:150 which is encoded by nucleotides 19-504 of SEQ ID NO:149; amino acid residues 7-167 of SEQ ID NO:150 which is encoded by nucleotides 19-501 of SEQ ID NO:149; amino acid residues 7-166 of SEQ ID NO:150 which is encoded by nucleotides 19-498 of SEQ ID NO:149; amino acid residues 8-176 of SEQ ID NO:150 which is encoded by nucleotides 22-528 of SEQ ID NO:149; amino acid residues 8-175 of SEQ ID NO:150 which is encoded by nucleotides 22-525 of SEQ ID NO:149; amino acid residues 8-174 of SEQ ID NO:150 which is encoded by nucleotides 22-522 of SEQ ID NO:149; amino acid residues 8-173 of SEQ ID NO:150 which is encoded by nucleotides 22-519 of SEQ ID NO:149; amino acid residues 8-172 of SEQ ID NO:150 which is encoded by nucleotides 22-516 of SEQ ID NO:149; amino acid residues 8-171 of SEQ ID NO:150 which is encoded by nucleotides 22-513 of SEQ ID NO:149; amino acid residues 8-170 of SEQ ID NO:150 which is encoded by nucleotides 22-510 of SEQ ID NO:149; amino acid residues 8-169 of SEQ ID NO:150 which is encoded by nucleotides 22-507 of SEQ ID NO:149; amino acid residues 8-168 of SEQ ID NO:150 which is encoded by nucleotides 22-504 of SEQ ID NO:149; amino acid residues 8-167 of SEQ ID NO:150 which is encoded by nucleotides 22-501 of SEQ ID NO:149; amino acid residues 8-166 of SEQ ID NO:150 which is encoded by nucleotides 22-498 of SEQ ID NO:149; amino acid residues 9-176 of SEQ ID NO:150 which is encoded by nucleotides 25-528 of SEQ ID NO:149; amino acid residues 9-175 of SEQ ID NO:150 which is encoded by nucleotides 25-525 of SEQ ID NO:149; amino acid residues 9-174 of SEQ ID NO:150 which is encoded by nucleotides 25-522 of SEQ ID NO:149; amino acid residues 9-173 of SEQ ID NO:150 which is encoded by nucleotides 25-519 of SEQ ID NO:149; amino acid residues 9-172 of SEQ ID NO:150 which is encoded by nucleotides 25-516 of SEQ ID NO:149; amino acid residues 9-171 of SEQ ID NO:150 which is encoded by nucleotides 25-513 of SEQ ID NO:149; amino acid residues 9-170 of SEQ ID NO:150 which is encoded by nucleotides 25-510 of SEQ ID NO:149; amino acid residues 9-169 of SEQ ID NO:150 which is encoded by nucleotides 25-507 of SEQ ID NO:149; amino acid residues 9-168 of SEQ ID NO:150 which is encoded by nucleotides 25-504 of SEQ ID NO:149; amino acid residues 9-167 of SEQ ID NO:150 which is encoded by nucleotides 25-501 of SEQ ID NO:149; amino acid residues 9-166 of SEQ ID NO:150 which is encoded by nucleotides 25-498 of SEQ ID NO:149; amino acid residues 10-176 of SEQ ID NO:150 which is encoded by nucleotides 28-528 of SEQ ID NO:149; amino acid residues 10-175 of SEQ ID NO:150 which is encoded by nucleotides 28-525 of SEQ ID NO:149; amino acid residues 10-174 of SEQ ID NO:150 which is encoded by nucleotides 28-522 of SEQ ID NO:149; amino acid residues 10-173 of SEQ ID NO:150 which is encoded by nucleotides 28-519 of SEQ ID NO:149; amino acid residues 10-172 of SEQ ID NO:150 which is encoded by nucleotides 28-516 of SEQ ID NO:149; amino acid residues 10-171 of SEQ ID NO:150 which is encoded by nucleotides 28-513 of SEQ ID NO:149; amino acid residues 10-170 of SEQ ID NO:150 which is encoded by nucleotides 28-510 of SEQ ID NO:149; amino acid residues 10-169 of SEQ ID NO:150 which is encoded by nucleotides 28-507 of SEQ ID NO:149; amino acid residues 10-168 of SEQ ID NO:150 which is encoded by nucleotides 28-504 of SEQ ID NO:149; amino acid residues 10-167 of SEQ ID NO:150 which is encoded by nucleotides 28-501 of SEQ ID NO:149; and amino acid residues 10-166 of SEQ ID NO:150 which is encoded by nucleotides 28-498 of SEQ ID NO:149. The N-terminally and C-terminally modified biologically active mutants of IL-29 C5 mutants of the present invention may also include an N-terminal Methione if expressed, for instance, in E. coli.

In addition to the IL-29 C5 mutants, the present invention also includes IL-29 polypeptides comprising a mutation at the first cysteine position, C1, of the mature polypeptide. For example, C1 from the N-terminus of the polypeptide of SEQ ID NO:20, is the cysteine at position 15, or position 16 (additional N-terminal Met) if expressed in E. coli (see, for example, SEQ ID NO:38). This first cysteine or C1 of IL-29 can be mutated, for example, to a serine, alanine, threonine, valine, or asparagines. These IL-29 C1 mutant polypeptides will thus have a predicted disulfide bond pattern of C2(Cys49 of SEQ ID NO:20)/C4(Cys145 of SEQ ID NO:20) and C3(Cys112 of SEQ ID NO:20)/C5(Cys171 of SEQ ID NO:20). Additional IL-29 C1 mutant molecules of the present invention include polynucleotide molecules as shown in SEQ ID NOs:41, 43, 45, 47, and 145, including DNA and RNA molecules, that encode IL-29 C1 mutant polypeptides as shown in SEQ ID NOs:42, 44, 46, 48 and 146, respectively. Additional IL-29 C1 mutant molecules of the present invention include polynucleotide molecules as shown in SEQ ID NOs:57, 59, 65, and 67, including DNA and RNA molecules, that encode IL-29 C1 mutant polypeptides as shown in SEQ ID NOs:58, 60, 66, and 68, respectively (PCT publication WO 03/066002 (Kotenko et al.)). Additional, IL-29 C1 mutant molecules of the present invention include polynucleotide molecules as shown in SEQ ID NOs:73, 75, 81, and 83, including DNA and RNA molecules, that encode IL-29 C1 mutant polypeptides as shown in SEQ ID NOs:74, 76, 82, and 84, respectively (PCT publication WO 02/092762 (Baum et al.)).

The present invention also includes biologically active mutants of IL-29 C1 cysteine mutants which provide, at least partially, an antiviral activity as provided here, e.g., anti-hepatitis C activity. The first cysteine or C1 from the N-terminus of IL-29 can mutated to any amino acid that does not form a disulfide bond with another cysteine, e.g., serine, alanine, threonine, valine or aspargine. The biologically active mutants of IL-29 C1 cysteine mutants of the present invention include N-, C-, and N- and C-terminal deletions of IL-29, e.g., the polypeptide of SEQ ID NOs:146 encoded by the polynucleotide of SEQ ID NO:145.

N-terminally modified biologically active mutants of IL-29 C1 mutants include, for example, amino acid residues 2-182 of SEQ ID NO:146 which is encoded by nucleotides 4-546 of SEQ ID NO:145; amino acid residues 3-182 of SEQ ID NO:146 which is encoded by nucleotides 7-546 of SEQ ID NO:145; amino acid residues 4-182 of SEQ ID NO:146 which is encoded by nucleotides 10-546 of SEQ ID NO:145; amino acid residues 5-182 of SEQ ID NO:146 which is encoded by nucleotides 13-546 of SEQ ID NO:145; amino acid residues 6-182 of SEQ ID NO:146 which is encoded by nucleotides 16-546 of SEQ ID NO:145; amino acid residues 7-182 of SEQ ID NO:146 which is encoded by nucleotides 19-546 of SEQ ID NO:145; amino acid residues 8-182 of SEQ ID NO:146 which is encoded by nucleotides 22-546 of SEQ ID NO:145; amino acid residues 9-182 of SEQ ID NO:146 which is encoded by nucleotides 25-546 of SEQ ID NO:145; amino acid residues 10-182 of SEQ ID NO:146 which is encoded by nucleotides 28-546 of SEQ ID NO:145; amino acid residues 11-182 of SEQ ID NO:146 which is encoded by nucleotides 31-546 of SEQ ID NO:145; amino acid residues 12-182 of SEQ ID NO:146 which is encoded by nucleotides 34-182 of SEQ ID NO:145; amino acid residues 13-182 of SEQ ID NO:146 which is encoded by nucleotides 37-546 of SEQ ID NO:145; amino acid residues 14-182 of SEQ ID NO:146 which is encoded by nucleotides 40-546 of SEQ ID NO:145; amino acid residues 15-182 of SEQ ID NO:146 which is encoded by nucleotides 43-546 of SEQ ID NO:145; and amino acid residues 16-182 of SEQ ID NO:146 which is encoded by nucleotides 46-546 of SEQ ID NO:145. The N-terminally modified biologically active mutants of IL-29 C1 mutants of the present invention may also include an N-terminal Methione if expressed, for instance, in E. coli.

C-terminally modified biologically active mutants of IL-29 C1 mutants include, for example, amino acid residues 1-181 of SEQ ID NO:146 which is encoded by nucleotides 1-543 of SEQ ID NO:145; amino acid residues 1-180 of SEQ ID NO:146 which is encoded by nucleotides 1-540 of SEQ ID NO:145; amino acid residues 1-179 of SEQ ID NO:146 which is encoded by nucleotides 1-537 of SEQ ID NO:145; amino acid residues 1-178 of SEQ ID NO:146 which is encoded by nucleotides 1-534 of SEQ ID NO:145; amino acid residues 1-177 of SEQ ID NO:146 which is encoded by nucleotides 1-531 of SEQ ID NO:145; amino acid residues 1-176 of SEQ ID NO:146 which is encoded by nucleotides 1-528 of SEQ ID NO:145; amino acid residues 1-175 of SEQ ID NO:146 which is encoded by nucleotides 1-525 of SEQ ID NO:145; amino acid residues 1-174 of SEQ ID NO:146 which is encoded by nucleotides 1-522 of SEQ ID NO:145; amino acid residues 1-173 of SEQ ID NO:146 which is encoded by nucleotides 1-519 of SEQ ID NO:145; and amino acid residues 1-172 of SEQ ID NO:146 which is encoded by nucleotides 1-516 of SEQ ID NO:145.

N-terminally and C-terminally modified biologically active mutants of IL-29 C1 mutants include, for example, amino acid residues 2-181 of SEQ ID NO:146 which is encoded by nucleotides 4-543 of SEQ ID NO:145; amino acid residues 2-180 of SEQ ID NO:146 which is encoded by nucleotides 4-540 of SEQ ID NO:145; amino acid residues 2-179 of SEQ ID NO:146 which is encoded by nucleotides 4-537 of SEQ ID NO:145; amino acid residues 2-178 of SEQ ID NO:146 which is encoded by nucleotides 4-534 of SEQ ID NO:145; amino acid residues 2-177 of SEQ ID NO:146 which is encoded by nucleotides 4-531 of SEQ ID NO:145; amino acid residues 2-176 of SEQ ID NO:146 which is encoded by nucleotides 4-528 of SEQ ID NO:145; amino acid residues 2-175 of SEQ ID NO:146 which is encoded by nucleotides 4-525 of SEQ ID NO:145; amino acid residues 2-174 of SEQ ID NO:146 which is encoded by nucleotides 4-522 of SEQ ID NO:145; amino acid residues 2-173 of SEQ ID NO:146 which is encoded by nucleotides 4-519 of SEQ ID NO:145; amino acid residues 2-172 of SEQ ID NO:146 which is encoded by nucleotides 4-516 of SEQ ID NO:145; amino acid residues 3-181 of SEQ ID NO:146 which is encoded by nucleotides 7-543 of SEQ ID NO:145; amino acid residues 3-180 of SEQ ID NO:146 which is encoded by nucleotides 7-540 of SEQ ID NO:145; amino acid residues 3-179 of SEQ ID NO:146 which is encoded by nucleotides 7-537 of SEQ ID NO:145; amino acid residues 3-178 of SEQ ID NO:146 which is encoded by nucleotides 7-534 of SEQ ID NO:145; amino acid residues 3-177 of SEQ ID NO:146 which is encoded by nucleotides 7-531 of SEQ ID NO:145; amino acid residues 3-176 of SEQ ID NO:146 which is encoded by nucleotides 7-528 of SEQ ID NO:145; amino acid residues 3-175 of SEQ ID NO:146 which is encoded by nucleotides 7-525 of SEQ ID NO:145; amino acid residues 3-174 of SEQ ID NO:146 which is encoded by nucleotides 7-522 of SEQ ID NO:145; amino acid residues 3-173 of SEQ ID NO:146 which is encoded by nucleotides 7-519 of SEQ ID NO:145; amino acid residues 3-172 of SEQ ID NO:146 which is encoded by nucleotides 7-516 of SEQ ID NO:145; amino acid residues 4-181 of SEQ ID NO:146 which is encoded by nucleotides 10-543 of SEQ ID NO:145; amino acid residues 4-180 of SEQ ID NO:146 which is encoded by nucleotides 10-540 of SEQ ID NO:145; amino acid residues 4-179 of SEQ ID NO:146 which is encoded by nucleotides 10-537 of SEQ ID NO:145; amino acid residues 4-178 of SEQ ID NO:146 which is encoded by nucleotides 10-534 of SEQ ID NO:145; amino acid residues 4-177 of SEQ ID NO:146 which is encoded by nucleotides 10-531 of SEQ ID NO:145; amino acid residues 4-176 of SEQ ID NO:146 which is encoded by nucleotides 10-528 of SEQ ID NO:145; amino acid residues 4-175 of SEQ ID NO:146 which is encoded by nucleotides 10-525 of SEQ ID NO:145; amino acid residues 4-174 of SEQ ID NO:146 which is encoded by nucleotides 10-522 of SEQ ID NO:145; amino acid residues 4-173 of SEQ ID NO:146 which is encoded by nucleotides 10-519 of SEQ ID NO:145; amino acid residues 4-172 of SEQ ID NO:146 which is encoded by nucleotides 10-516 of SEQ ID NO:145; amino acid residues 5-181 of SEQ ID NO:146 which is encoded by nucleotides 13-543 of SEQ ID NO:145; amino acid residues 5-180 of SEQ ID NO:146 which is encoded by nucleotides 13-540 of SEQ ID NO:145; amino acid residues 5-179 of SEQ ID NO:146 which is encoded by nucleotides 13-537 of SEQ ID NO:145; amino acid residues 5-178 of SEQ ID NO:146 which is encoded by nucleotides 13-534 of SEQ ID NO:145; amino acid residues 5-177 of SEQ ID NO:146 which is encoded by nucleotides 13-531 of SEQ ID NO:145; amino acid residues 5-176 of SEQ ID NO:146 which is encoded by nucleotides 13-528 of SEQ ID NO:145; amino acid residues 5-175 of SEQ ID NO:146 which is encoded by nucleotides 13-525 of SEQ ID NO:145; amino acid residues 5-174 of SEQ ID NO:146 which is encoded by nucleotides 13-522 of SEQ ID NO:145; amino acid residues 5-173 of SEQ ID NO:146 which is encoded by nucleotides 13-519 of SEQ ID NO:145; amino acid residues 5-172 of SEQ ID NO:146 which is encoded by nucleotides 13-516 of SEQ ID NO:145; amino acid residues 6-181 of SEQ ID NO:146 which is encoded by nucleotides 16-543 of SEQ ID NO:145; amino acid residues 6-180 of SEQ ID NO:146 which is encoded by nucleotides 16-540 of SEQ ID NO:145; amino acid residues 6-179 of SEQ ID NO:146 which is encoded by nucleotides 16-537 of SEQ ID NO:145; amino acid residues 6-178 of SEQ ID NO:146 which is encoded by nucleotides 16-534 of SEQ ID NO:145; amino acid residues 6-177 of SEQ ID NO:146 which is encoded by nucleotides 16-531 of SEQ ID NO:145; amino acid residues 6-176 of SEQ ID NO:146 which is encoded by nucleotides 16-528 of SEQ ID NO:145; amino acid residues 6-175 of SEQ ID NO:146 which is encoded by nucleotides 16-525 of SEQ ID NO:145; amino acid residues 6-174 of SEQ ID NO:146 which is encoded by nucleotides 16-522 of SEQ ID NO:145; amino acid residues 6-173 of SEQ ID NO:146 which is encoded by nucleotides 16-519 of SEQ ID NO:145; amino acid residues 6-172 of SEQ ID NO:146 which is encoded by nucleotides 16-516 of SEQ ID NO:145; amino acid residues 7-181 of SEQ ID NO:146 which is encoded by nucleotides 19-543 of SEQ ID NO:145; amino acid residues 7-180 of SEQ ID NO:146 which is encoded by nucleotides 19-540 of SEQ ID NO:145; amino acid residues 7-179 of SEQ ID NO:146 which is encoded by nucleotides 19-537 of SEQ ID NO:145; amino acid residues 7-178 of SEQ ID NO:146 which is encoded by nucleotides 19-534 of SEQ ID NO:145; amino acid residues 7-177 of SEQ ID NO:146 which is encoded by nucleotides 19-531 of SEQ ID NO:145; amino acid residues 7-176 of SEQ ID NO:146 which is encoded by nucleotides 19-528 of SEQ ID NO:145; amino acid residues 7-175 of SEQ ID NO:146 which is encoded by nucleotides 19-525 of SEQ ID NO:145; amino acid residues 7-174 of SEQ ID NO:146 which is encoded by nucleotides 19-522 of SEQ ID NO:145; amino acid residues 7-173 of SEQ ID NO:146 which is encoded by nucleotides 19-519 of SEQ ID NO:145; amino acid residues 7-172 of SEQ ID NO:146 which is encoded by nucleotides 19-516 of SEQ ID NO:145; amino acid residues 8-181 of SEQ ID NO:146 which is encoded by nucleotides 22-543 of SEQ ID NO:145; amino acid residues 8-180 of SEQ ID NO:146 which is encoded by nucleotides 22-540 of SEQ ID NO:145; amino acid residues 8-179 of SEQ ID NO:146 which is encoded by nucleotides 22-537 of SEQ ID NO:145; amino acid residues 8-178 of SEQ ID NO:146 which is encoded by nucleotides 22-534 of SEQ ID NO:145; amino acid residues 8-177 of SEQ ID NO:146 which is encoded by nucleotides 22-531 of SEQ ID NO:145; amino acid residues 8-176 of SEQ ID NO:146 which is encoded by nucleotides 22-528 of SEQ ID NO:145; amino acid residues 8-175 of SEQ ID NO:146 which is encoded by nucleotides 22-525 of SEQ ID NO:145; amino acid residues 8-174 of SEQ ID NO:146 which is encoded by nucleotides 22-522 of SEQ ID NO:145; amino acid residues 8-173 of SEQ ID NO:146 which is encoded by nucleotides 22-519 of SEQ ID NO:145; amino acid residues 8-172 of SEQ ID NO:146 which is encoded by nucleotides 22-516 of SEQ ID NO:145; amino acid residues 9-181 of SEQ ID NO:146 which is encoded by nucleotides 25-543 of SEQ ID NO:145; amino acid residues 9-180 of SEQ ID NO:146 which is encoded by nucleotides 25-540 of SEQ ID NO:145; amino acid residues 9-179 of SEQ ID NO:146 which is encoded by nucleotides 25-537 of SEQ ID NO:145; amino acid residues 9-178 of SEQ ID NO:146 which is encoded by nucleotides 25-534 of SEQ ID NO:145; amino acid residues 9-177 of SEQ ID NO:146 which is encoded by nucleotides 25-531 of SEQ ID NO:145; amino acid residues 9-176 of SEQ ID NO:146 which is encoded by nucleotides 25-528 of SEQ ID NO:145; amino acid residues 9-175 of SEQ ID NO:146 which is encoded by nucleotides 25-525 of SEQ ID NO:145; amino acid residues 9-174 of SEQ ID NO:146 which is encoded by nucleotides 25-522 of SEQ ID NO:145; amino acid residues 9-173 of SEQ ID NO:146 which is encoded by nucleotides 25-519 of SEQ ID NO:145; amino acid residues 9-172 of SEQ ID NO:146 which is encoded by nucleotides 25-516 of SEQ ID NO:145; amino acid residues 10-181 of SEQ ID NO:146 which is encoded by nucleotides 28-543 of SEQ ID NO:145; amino acid residues 10-180 of SEQ ID NO:146 which is encoded by nucleotides 28-540 of SEQ ID NO:145; amino acid residues 10-179 of SEQ ID NO:146 which is encoded by nucleotides 28-537 of SEQ ID NO:145; amino acid residues 10-178 of SEQ ID NO:146 which is encoded by nucleotides 28-534 of SEQ ID NO:145; amino acid residues 10-177 of SEQ ID NO:146 which is encoded by nucleotides 28-531 of SEQ ID NO:145; amino acid residues 10-176 of SEQ ID NO:146 which is encoded by nucleotides 28-528 of SEQ ID NO:145; amino acid residues 10-175 of SEQ ID NO:146 which is encoded by nucleotides 28-525 of SEQ ID NO:145; amino acid residues 10-174 of SEQ ID NO:146 which is encoded by nucleotides 28-522 of SEQ ID NO:145; amino acid residues 10-173 of SEQ ID NO:146 which is encoded by nucleotides 28-519 of SEQ ID NO:145; amino acid residues 10-172 of SEQ ID NO:146 which is encoded by nucleotides 28-516 of SEQ ID NO:145; amino acid residues 11-181 of SEQ ID NO:146 which is encoded by nucleotides 31-543 of SEQ ID NO:145; amino acid residues 11-180 of SEQ ID NO:146 which is encoded by nucleotides 31-540 of SEQ ID NO:145; amino acid residues 11-179 of SEQ ID NO:146 which is encoded by nucleotides 31-537 of SEQ ID NO:145; amino acid residues 11-178 of SEQ ID NO:146 which is encoded by nucleotides 31-534 of SEQ ID NO:145; amino acid residues 11-177 of SEQ ID NO:146 which is encoded by nucleotides 31-531 of SEQ ID NO:145; amino acid residues 11-176 of SEQ ID NO:146 which is encoded by nucleotides 31-528 of SEQ ID NO:145; amino acid residues 11-175 of SEQ ID NO:146 which is encoded by nucleotides 31-525 of SEQ ID NO:145; amino acid residues 11-174 of SEQ ID NO:146 which is encoded by nucleotides 31-522 of SEQ ID NO:145; amino acid residues 11-173 of SEQ ID NO:146 which is encoded by nucleotides 31-519 of SEQ ID NO:145; amino acid residues 11-172 of SEQ ID NO:146 which is encoded by nucleotides 31-516 of SEQ ID NO:145; amino acid residues 12-181 of SEQ ID NO:146 which is encoded by nucleotides 34-543 of SEQ ID NO:145; amino acid residues 12-180 of SEQ ID NO:146 which is encoded by nucleotides 34-540 of SEQ ID NO:145; amino acid residues 12-179 of SEQ ID NO:146 which is encoded by nucleotides 34-537 of SEQ ID NO:145; amino acid residues 12-178 of SEQ ID NO:146 which is encoded by nucleotides 34-534 of SEQ ID NO:145; amino acid residues 12-177 of SEQ ID NO:146 which is encoded by nucleotides 34-531 of SEQ ID NO:145; amino acid residues 12-176 of SEQ ID NO:146 which is encoded by nucleotides 34-528 of SEQ ID NO:145; amino acid residues 12-175 of SEQ ID NO:146 which is encoded by nucleotides 34-525 of SEQ ID NO:145; amino acid residues 12-174 of SEQ ID NO:146 which is encoded by nucleotides 34-522 of SEQ ID NO:145; amino acid residues 12-173 of SEQ ID NO:146 which is encoded by nucleotides 34-519 of SEQ ID NO:145; amino acid residues 12-172 of SEQ ID NO:146 which is encoded by nucleotides 34-516 of SEQ ID NO:145; amino acid residues 13-181 of SEQ ID NO:146 which is encoded by nucleotides 37-543 of SEQ ID NO:145; amino acid residues 13-180 of SEQ ID NO:146 which is encoded by nucleotides 37-540 of SEQ ID NO:145; amino acid residues 13-179 of SEQ ID NO:146 which is encoded by nucleotides 37-537 of SEQ ID NO:145; amino acid residues 13-178 of SEQ ID NO:146 which is encoded by nucleotides 37-534 of SEQ ID NO:145; amino acid residues 13-177 of SEQ ID NO:146 which is encoded by nucleotides 37-531 of SEQ ID NO:145; amino acid residues 13-176 of SEQ ID NO:146 which is encoded by nucleotides 37-528 of SEQ ID NO:145; amino acid residues 13-175 of SEQ ID NO:146 which is encoded by nucleotides 37-525 of SEQ ID NO:145; amino acid residues 13-174 of SEQ ID NO:146 which is encoded by nucleotides 37-522 of SEQ ID NO:145; amino acid residues 13-173 of SEQ ID NO:146 which is encoded by nucleotides 37-519 of SEQ ID NO:145; amino acid residues 13-172 of SEQ ID NO:146 which is encoded by nucleotides 37-516 of SEQ ID NO:145; amino acid residues 14-181 of SEQ ID NO:146 which is encoded by nucleotides 40-543 of SEQ ID NO:145; amino acid residues 14-180 of SEQ ID NO:146 which is encoded by nucleotides 40-540 of SEQ ID NO:145; amino acid residues 14-179 of SEQ ID NO:146 which is encoded by nucleotides 40-537 of SEQ ID NO:145; amino acid residues 14-178 of SEQ ID NO:146 which is encoded by nucleotides 40-534 of SEQ ID NO:145; amino acid residues 14-177 of SEQ ID NO:146 which is encoded by nucleotides 40-531 of SEQ ID NO:145; amino acid residues 14-176 of SEQ ID NO:146 which is encoded by nucleotides 40-528 of SEQ ID NO:145; amino acid residues 14-175 of SEQ ID NO:146 which is encoded by nucleotides 40-525 of SEQ ID NO:145; amino acid residues 14-174 of SEQ ID NO:146 which is encoded by nucleotides 40-522 of SEQ ID NO:145; amino acid residues 14-173 of SEQ ID NO:146 which is encoded by nucleotides 40-519 of SEQ ID NO:145; amino acid residues 14-172 of SEQ ID NO:146 which is encoded by nucleotides 40-516 of SEQ ID NO:145; amino acid residues 15-181 of SEQ ID NO:146 which is encoded by nucleotides 43-543 of SEQ ID NO:145; amino acid residues 15-180 of SEQ ID NO:146 which is encoded by nucleotides 43-540 of SEQ ID NO:145; amino acid residues 15-179 of SEQ ID NO:146 which is encoded by nucleotides 43-537 of SEQ ID NO:145; amino acid residues 15-178 of SEQ ID NO:146 which is encoded by nucleotides 43-534 of SEQ ID NO:145; amino acid residues 15-177 of SEQ ID NO:146 which is encoded by nucleotides 43-531 of SEQ ID NO:145; amino acid residues 15-176 of SEQ ID NO:146 which is encoded by nucleotides 43-528 of SEQ ID NO:145; amino acid residues 15-175 of SEQ ID NO:146 which is encoded by nucleotides 43-525 of SEQ ID NO:145; amino acid residues 15-174 of SEQ ID NO:146 which is encoded by nucleotides 43-522 of SEQ ID NO:145; amino acid residues 15-173 of SEQ ID NO:146 which is encoded by nucleotides 43-519 of SEQ ID NO:145; amino acid residues 15-172 of SEQ ID NO:146 which is encoded by nucleotides 43-516 of SEQ ID NO:145; amino acid residues 16-181 of SEQ ID NO:146 which is encoded by nucleotides 46-543 of SEQ ID NO:145; amino acid residues 16-180 of SEQ ID NO:146 which is encoded by nucleotides 46-540 of SEQ ID NO:145; amino acid residues 16-179 of SEQ ID NO:146 which is encoded by nucleotides 46-537 of SEQ ID NO:145; amino acid residues 16-178 of SEQ ID NO:146 which is encoded by nucleotides 46-534 of SEQ ID NO:145; amino acid residues 16-177 of SEQ ID NO:146 which is encoded by nucleotides 46-531 of SEQ ID NO:145; amino acid residues 16-176 of SEQ ID NO:146 which is encoded by nucleotides 46-528 of SEQ ID NO:145; amino acid residues 16-175 of SEQ ID NO:146 which is encoded by nucleotides 46-525 of SEQ ID NO:145; amino acid residues 16-174 of SEQ ID NO:146 which is encoded by nucleotides 46-522 of SEQ ID NO:145; amino acid residues 16-173 of SEQ ID NO:146 which is encoded by nucleotides 46-519 of SEQ ID NO:145; and amino acid residues 16-172 of SEQ ID NO:146 which is encoded by nucleotides 46-516 of SEQ ID NO:145. The N-terminally and C-terminally modified biologically active mutants of IL-29 C1 mutants of the present invention may also include an N-terminal Methione if expressed, for instance, in E. coli.

The IL-29 polypeptides of the present invention include, for example, SEQ ID NOs:4, 20, 32, 34, 38, 42, 44, 46, 48, 50, 52, 54, 56, 58, 60, 62, 64, 66, 68, 70, 72, 74, 76, 78, 80, 82, 84, 110, 112, 114, 116, 118, 120, 122, 124, 126, 128, 130, 132, 134, 136, 146, 148, 150, and biologically active mutants, fusions, variants and fragments thereof which are encoded by IL-29 polynucleotide molecules as shown in SEQ ID NOs:3, 19, 31, 33, 37, 41, 43, 45, 47, 49, 51, 53, 55, 57, 59, 61, 63, 65, 67, 69, 71, 73, 75, 77, 79, 81, 83, 109, 111, 113, 115, 117, 119, 121, 123, 125, 127, 129, 131, 133, 135, 145, 147 and 149, respectively, may further include a signal sequence as shown in SEQ ID NOs:102, 104, 106, or 108. A polynucleotide molecule encoding the signal sequence polypeptides of SEQ ID NOs:102, 104, 106, and 108 are shown as SEQ ID NOs:101, 103, 105, and 107, respectively.

An IL-28B gene encodes a polypeptide of 205 amino acids, as shown in SEQ ID NO:6. The signal sequence for IL-28B comprises amino acid residue 1 (Met) through amino acid residue 21 (Ala) of SEQ ID NO:6. The mature peptide for IL-28B begins at amino acid residue 22 (Val). A variant IL-28B gene encodes a polypeptide of 200 amino acids, as shown in SEQ ID NO:22. The signal sequence for IL-28B can be predicted as comprising amino acid residue −25 (Met) through amino acid residue −1 (Ala) of SEQ ID NO:22. The mature peptide for IL-28B begins at amino acid residue 1 (Val) of SEQ ID NO:22. IL-28B helices are predicted as follow: helix A is defined by amino acid residues 31 (Ala) to 45 (Leu); helix B by amino acid residues 58 (Thr) to 65 (Gln); helix C by amino acid residues 69 (Arg) to 86 (Ala); helix D by amino acid residues 95 (Gly) to 114 (Ala); helix E by amino acid residues 126 (Thr) to 142 (Lys); and helix F by amino acid residues 148 (Cys) to 169 (Ala); as shown in SEQ ID NO:22. When a polynucleotide sequence encoding the mature polypeptide is expressed in a prokaryotic system, such as E. coli, a secretory signal sequence may not be required and an N-terminal Met may present, resulting in expression of a polypeptide such as is shown in SEQ ID NO:40.

IL-28B polypeptides of the present invention also include a mutation at the second cysteine, C2, of the mature polypeptide. For example, C2 from the N-terminus of the polypeptide of SEQ ID NO:22 is the cysteine at amino acid position 48, or position 49 (additional N-terminal Met) if expressed in E. coli (see, for example, SEQ ID NO:40). This second cysteine (of which there are seven, like IL-28A) or C2 of IL-28B can be mutated, for example, to a serine, alanine, threonine, valine, or asparagine. IL-28B C2 mutant molecules of the present invention include, for example, polynucleotide molecules as shown in SEQ ID NOs:85, 87 and 141, including DNA and RNA molecules, that encode IL-28B C2 mutant polypeptides as shown in SEQ ID NOs:86, 88 and 142, respectively. Additional IL-28B C2 mutant molecules of the present invention include polynucleotide molecules as shown in SEQ ID NOs:93 and 95 including DNA and RNA molecules, that encode IL-28 C2 mutant polypeptides as shown in SEQ ID NOs:94 and 96, respectively (PCT publication WO 03/066002 (Kotenko et al.)).

The present invention also includes biologically active mutants of IL-28B C2 cysteine mutants which provide, at least partially, an antiviral activity as provided here, e.g., anti-hepatitis C activity. The second cysteine or C2 from the N-terminus of IL-28B can mutated to any amino acid that does not form a disulfide bond with another cysteine, e.g., serine, alanine, threonine, valine or aspargine. The biologically active mutants of IL-28B C2 cysteine mutants of the present invention include N-, C-, and N- and C-terminal deletions of IL-28B, e.g., the polypeptide of SEQ ID NO:142 encoded by the polynucleotide of SEQ ID NO:141.

N-terminally modified biologically active mutants of IL-28B C2 mutants include, for example, amino acid residues 2-176 of SEQ ID NO:142 which is encoded by nucleotides 4-528 of SEQ ID NO:141; amino acid residues 3-176 of SEQ ID NO:142 which is encoded by nucleotides 7-528 of SEQ ID NO:141; amino acid residues 4-176 of SEQ ID NO:142 which is encoded by nucleotides 10-528 of SEQ ID NO:141; amino acid residues 5-176 of SEQ ID NO:142 which is encoded by nucleotides 13-528 of SEQ ID NO:141; amino acid residues 6-176 of SEQ ID NO:142 which is encoded by nucleotides 16-528 of SEQ ID NO:141; amino acid residues 7-176 of SEQ ID NO:142 which is encoded by nucleotides 19-528 of SEQ ID NO:141; amino acid residues 8-176 of SEQ ID NO:142 which is encoded by nucleotides 22-528 of SEQ ID NO:141; amino acid residues 9-176 of SEQ ID NO:142 which is encoded by nucleotides 25-528 of SEQ ID NO:141; amino acid residues 10-176 of SEQ ID NO:142 which is encoded by nucleotides 28-528 of SEQ ID NO:141; amino acid residues 11-176 of SEQ ID NO:142 which is encoded by nucleotides 31-528 of SEQ ID NO:141; amino acid residues 12-176 of SEQ ID NO:142 which is encoded by nucleotides 34-528 of SEQ ID NO:141; amino acid residues 13-176 of SEQ ID NO:142 which is encoded by nucleotides 37-528 of SEQ ID NO:141; amino acid residues 14-176 of SEQ ID NO:142 which is encoded by nucleotides 40-528 of SEQ ID NO:141; amino acid residues 15-176 of SEQ ID NO:142 which is encoded by nucleotides 43-528 of SEQ ID NO:141; amino acid residues 16-176 of SEQ ID NO:142 which is encoded by nucleotides 46-528 of SEQ ID NO:141; and amino acid residues 17-176 of SEQ ID NO:142 which is encoded by nucleotides 49-528 of SEQ ID NO:141. The N-terminally modified biologically active mutants of IL-28 C2 mutants of the present invention may also include an N-terminal Methione if expressed, for instance, in E. coli.

C-terminally modified biologically active mutants of IL-28B C2 mutants include, for example, amino acid residues 1-175 of SEQ ID NO:142 which is encoded by nucleotides 1-525 of SEQ ID NO:141.

N-terminally and C-terminally biologically active mutants of IL-28B C2 mutants include, for example, amino acid residues 2-175 of SEQ ID NO:142 which is encoded by nucleotides 4-525 of SEQ ID NO:141; amino acid residues 3-175 of SEQ ID NO:142 which is encoded by nucleotides 7-525 of SEQ ID NO:141; amino acid residues 4-175 of SEQ ID NO:142 which is encoded by nucleotides 10-525 of SEQ ID NO:141; amino acid residues 5-175 of SEQ ID NO:142 which is encoded by nucleotides 13-525 of SEQ ID NO:141; amino acid residues 6-175 of SEQ ID NO:142 which is encoded by nucleotides 16-525 of SEQ ID NO:141; amino acid residues 7-175 of SEQ ID NO:142 which is encoded by nucleotides 19-525 of SEQ ID NO:141; amino acid residues 8-175 of SEQ ID NO:142 which is encoded by nucleotides 22-525 of SEQ ID NO:141; amino acid residues 9-175 of SEQ ID NO:142 which is encoded by nucleotides 25-525 of SEQ ID NO:141; amino acid residues 10-175 of SEQ ID NO:142 which is encoded by nucleotides 28-525 of SEQ ID NO:141; amino acid residues 11-175 of SEQ ID NO:142 which is encoded by nucleotides 31-525 of SEQ ID NO:141; amino acid residues 12-175 of SEQ ID NO:142 which is encoded by nucleotides 34-525 of SEQ ID NO:141; amino acid residues 13-175 of SEQ ID NO:142 which is encoded by nucleotides 37-525 of SEQ ID NO:141; amino acid residues 14-175 of SEQ ID NO:142 which is encoded by nucleotides 40-525 of SEQ ID NO:141; amino acid residues 15-175 of SEQ ID NO:142 which is encoded by nucleotides 43-525 of SEQ ID NO:141; amino acid residues 16-175 of SEQ ID NO:142 which is encoded by nucleotides 46-525 of SEQ ID NO:141; and amino acid residues 17-175 of SEQ ID NO:142 which is encoded by nucleotides 49-525 of SEQ ID NO:141. The N-terminally and C-terminally modified biologically active mutants of IL-28 C2 mutants of the present invention may also include an N-terminal Methione if expressed, for instance, in E. coli.

In addition to the IL-28B C2 mutants, the present invention also includes IL-28B polypeptides comprising a mutation at the third cysteine position, C3, of the mature polypeptide. For example, C3 from the N-terminus of the polypeptide of SEQ ID NO:22, is the cysteine at position 50, or position 51 (additional N-terminal Met) if expressed in E. coli (see, for example, SEQ ID NO:40). IL-28B C3 mutant molecules of the present invention include, for example, polynucleotide molecules as shown in SEQ ID NOs:89, 91 and 143, including DNA and RNA molecules, that encode IL-28B C3 mutant polypeptides as shown in SEQ ID NOs:90, 92 and 144, respectively. Additional IL-28B C3 mutant molecules of the present invention include polynucleotide molecules as shown in SEQ ID NOs:97 and 99 including DNA and RNA molecules, that encode IL-28B C3 mutant polypeptides as shown in SEQ ID NOs:98 and 100, respectively (PCT publication WO 03/066002 (Kotenko et al.)).

N-terminally biologically active mutants of IL-28B C3 mutants include, for example, amino acid residues 2-176 of SEQ ID NO:144 which is encoded by nucleotides 4-528 of SEQ ID NO:143; amino acid residues 3-176 of SEQ ID NO:144 which is encoded by nucleotides 7-528 of SEQ ID NO:143; amino acid residues 4-176 of SEQ ID NO:144 which is encoded by nucleotides 10-528 of SEQ ID NO:143; amino acid residues 5-176 of SEQ ID NO:144 which is encoded by nucleotides 13-528 of SEQ ID NO:143; amino acid residues 6-176 of SEQ ID NO:144 which is encoded by nucleotides 16-528 of SEQ ID NO:143; amino acid residues 7-176 of SEQ ID NO:144 which is encoded by nucleotides 19-528 of SEQ ID NO:143; amino acid residues 8-176 of SEQ ID NO:144 which is encoded by nucleotides 22-528 of SEQ ID NO:143; amino acid residues 9-176 of SEQ ID NO:144 which is encoded by nucleotides 25-528 of SEQ ID NO:143; amino acid residues 10-176 of SEQ ID NO:144 which is encoded by nucleotides 28-528 of SEQ ID NO:143; amino acid residues 11-176 of SEQ ID NO:144 which is encoded by nucleotides 31-528 of SEQ ID NO:143; amino acid residues 12-176 of SEQ ID NO:144 which is encoded by nucleotides 34-528 of SEQ ID NO:143; amino acid residues 13-176 of SEQ ID NO:144 which is encoded by nucleotides 37-528 of SEQ ID NO:143; amino acid residues 14-176 of SEQ ID NO:144 which is encoded by nucleotides 40-528 of SEQ ID NO:143; amino acid residues 15-176 of SEQ ID NO:144 which is encoded by nucleotides 43-528 of SEQ ID NO:143; amino acid residues 16-176 of SEQ ID NO:144 which is encoded by nucleotides 46-528 of SEQ ID NO:143; and amino acid residues 17-176 of SEQ ID NO:144 which is encoded by nucleotides 49-528 of SEQ ID NO:143. The N-terminally modified biologically active mutants of IL-28 C3 mutants of the present invention may also include an N-terminal Methione if expressed, for instance, in E. coli.

C-terminally modified biologically active mutants of IL-28B C3 mutants include, for example, amino acid residues 1-175 of SEQ ID NO:144 which is encoded by nucleotides 1-525 of SEQ ID NO:143.

N-terminally and C-terminally biologically active mutants of IL-28B C3 mutants include, for example, amino acid residues 2-175 of SEQ ID NO:144 which is encoded by nucleotides 4-525 of SEQ ID NO:143; amino acid residues 3-175 of SEQ ID NO:144 which is encoded by nucleotides 7-525 of SEQ ID NO:143; amino acid residues 4-175 of SEQ ID NO:144 which is encoded by nucleotides 10-525 of SEQ ID NO:143; amino acid residues 5-175 of SEQ ID NO:144 which is encoded by nucleotides 13-525 of SEQ ID NO:143; amino acid residues 6-175 of SEQ ID NO:144 which is encoded by nucleotides 16-525 of SEQ ID NO:143; amino acid residues 7-175 of SEQ ID NO:144 which is encoded by nucleotides 19-525 of SEQ ID NO:143; amino acid residues 8-175 of SEQ ID NO:144 which is encoded by nucleotides 22-525 of SEQ ID NO:143; amino acid residues 9-175 of SEQ ID NO:144 which is encoded by nucleotides 25-525 of SEQ ID NO:143; amino acid residues 10-175 of SEQ ID NO:144 which is encoded by nucleotides 28-525 of SEQ ID NO:143; amino acid residues 11-175 of SEQ ID NO:144 which is encoded by nucleotides 31-525 of SEQ ID NO:143; amino acid residues 12-175 of SEQ ID NO:144 which is encoded by nucleotides 34-525 of SEQ ID NO:143; amino acid residues 13-175 of SEQ ID NO:144 which is encoded by nucleotides 37-525 of SEQ ID NO:143; amino acid residues 14-175 of SEQ ID NO:144 which is encoded by nucleotides 40-525 of SEQ ID NO:143; amino acid residues 15-175 of SEQ ID NO:144 which is encoded by nucleotides 43-525 of SEQ ID NO:143; amino acid residues 16-175 of SEQ ID NO:144 which is encoded by nucleotides 46-525 of SEQ ID NO:143; and amino acid residues 17-175 of SEQ ID NO:144 which is encoded by nucleotides 49-525 of SEQ ID NO:143. The N-terminally and C-terminally modified biologically active mutants of IL-28 C3 mutants of the present invention may also include an N-terminal Methione if expressed, for instance, in E. coli.

The IL-28B polypeptides of the present invention include, for example, SEQ ID NOs:6, 22, 40, 86, 88, 90, 92, 94, 96, 98, 100, 142, 144, and biologically active mutants, fusions, variants and fragments thereof which are encoded by IL-28B polynucleotide molecules as shown in SEQ ID NOs:5, 21, 39, 85, 87, 89, 91, 93, 95, 97, 99, 141 and 143, respectively.

Zcyto24 gene encodes a polypeptide of 202 amino acids, as shown in SEQ ID NO:8. Zcyto24 secretory signal sequence comprises amino acid residue 1 (Met) through amino acid residue 28 (Ala) of SEQ ID NO:8. An alternative site for cleavage of the secretory signal sequence can be found at amino acid residue 24 (Thr). The mature polypeptide comprises amino acid residue 29 (Asp) to amino acid residue 202 (Val).

Zcyto25 gene encodes a polypeptide of 202 amino acids, as shown in SEQ ID NO:10. Zcyto25 secretory signal sequence comprises amino acid residue 1 (Met) through amino acid residue 28 (Ala) of SEQ ID NO:10. An alternative site for cleavage of the secretory signal sequence can be found at amino acid residue 24 (Thr). The mature polypeptide comprises amino acid residue 29 (Asp) to amino acid residue 202 (Val).

The IL-28 and IL-29 cysteine mutant polypeptides of the present invention provided for the expression of a single-disulfide form of the IL-28 or IL-29 molecule. When IL-28 and IL-29 are expressed in E. coli, an N-terminal Methionine is present. SEQ ID NOs:26, and 34, for instance, show the amino acid residue numbering for IL-28A and IL-29 mutants, respectively, when the N-terminal Met is present. Table 1 shows the possible combinations of intramolecular disulfide bonded cysteine pairs for wildtype IL-28A, IL-28B, and IL-29.

TABLE 1

| | | | | | | | | | |
|---|---|---|---|---|---|---|---|---|---|
| IL-28A | C₁₆-C₁₁₅ | C₄₈-C₁₄₈ | C₅₀-C₁₄₈ | C₁₆₇-C₁₇₄ | C₁₆-C₄₈ | C₁₆-C₅₀ | C₄₈-C₁₁₅ | C₅₀-C₁₁₅ | C₁₁₅-C₁₄₈ |
| SEQ ID | | | | | | | | | |
| NO: 18 | | | | | | | | | |
| Met IL- | C₁₇-C₁₁₆ | C₄₉-C₁₄₉ | C₅₁-C₁₄₉₈ | C₁₆₈-C₁₇₅ | C₁₇-C₄₉ | C₁₇-C₅₁ | C₄₉-C₁₁₆ | C₅₁-C₁₁₆ | C₁₁₆-C₁₄₉ |
| 28A | | | | | | | | | |
| SEQ ID | | | | | | | | | |
| NO: 36 | | | | | | | | | |
| IL-29 | C₁₅-C₁₁₂ | C₄₉-C₁₄₅ | C₁₁₂-C₁₇₁ | | | | | | |
| SEQ ID | | | | | | | | | |
| NO: 20 | | | | | | | | | |
| Met IL-29 | C₁₆-C₁₁₃ | C₅₀-C₁₄₆ | C₁₁₃-C₁₇₂ | | | | | | |
| SEQ ID | | | | | | | | | |
| NO: 38 | | | | | | | | | |
| IL-28B | C₁₆-C₁₁₅ | C₄₈-C₁₄₈ | C₅₀-C₁₄₈ | C₁₆₇-C₁₇₄ | C₁₆-C₄₈ | C₁₆-C₅₀ | C₄₈-C₁₁₅ | C₅₀-C₁₁₅ | C₁₁₅-C₁₄₈ |
| SEQ ID | | | | | | | | | |
| NO: 22 | | | | | | | | | |
| Met IL-28B | C₁₇-C₁₁₆ | C₄₉-C₁₄₉ | C₅₁-C₁₄₉₈ | C₁₆₈-C₁₇₅ | C₁₇-C₄₉ | C₁₇-C₅₁ | C₄₉-C₁₁₆ | C₅₁-C₁₁₆ | C₁₁₆-C₁₄₉ |
| SEQ ID | | | | | | | | | |
| NO: 40 | | | | | | | | | |

Using methods known in the art, IL-28 or IL-29 polypeptides of the present invention can be prepared as monomers or multimers; glycosylated or non-glycosylated; pegylated or non-pegylated; fusion proteins; and may or may not include an initial methionine amino acid residue. IL-28 or IL-29 polypeptides can be conjugated to acceptable water-soluble polymer moieties for use in therapy. Conjugation of interferons, for example, with water-soluble polymers has been shown to enhance the circulating half-life of the interferon, and to reduce the immunogenicity of the polypeptide (see, for example, Nieforth et al., Clin. Pharmacol. Ther. 59:636 (1996), and Monkarsh et al., Anal. Biochem. 247:434 (1997)).

Suitable water-soluble polymers include polyethylene glycol (PEG), monomethoxy-PEG, mono-(C1-C10)alkoxy-PEG, aryloxy-PEG, poly-(N-vinyl pyrrolidone)PEG, tresyl monomethoxy PEG, monomethoxy-PEG propionaldehyde, PEG propionaldehyde, bis-succinimidyl carbonate PEG, propylene glycol homopolymers, a polypropylene oxide/ethylene oxide co-polymer, polyoxyethylated polyols (e.g., glycerol), monomethoxy-PEG butyraldehyde, PEG butyraldehyde, monomethoxy-PEG acetaldehyde, PEG acetaldehyde, methoxyl PEG-succinimidyl propionate, methoxyl PEG-succinimidyl butanoate, polyvinyl alcohol, dextran, cellulose, or other carbohydrate-based polymers. Suitable PEG may have a molecular weight from about 600 to about 60,000, including, for example, 5,000, 12,000, 20,000, 30,000, 40,000, and 50,000, which can be linear or branched. A IL-28 or IL-29 conjugate can also comprise a mixture of such water-soluble polymers.

One example of an IL-28 or IL-29 conjugate comprises an IL-28 or IL-29 moiety and a polyalkyl oxide moiety attached to the N-terminus of the IL-28 or IL-29 moiety. PEG is one suitable polyalkyl oxide. As an illustration, IL-28 or IL-29 can be modified with PEG, a process known as “PEGylation.” PEGylation of an IL-28 or IL-29 can be carried out by any of the PEGylation reactions known in the art (see, for example, EP 0 154 316, Delgado et al., Critical Reviews in Therapeutic Drug Carrier Systems 9:249 (1992), Duncan and Spreafico, Clin. Pharmacokinet. 27:290 (1994), and Francis et al., Int J Hematol 68:1 (1998)). For example, PEGylation can be performed by an acylation reaction or by an alkylation reaction with a reactive polyethylene glycol molecule. In an alternative approach, IL-28 or IL-29 conjugates are formed by condensing activated PEG, in which a terminal hydroxy or amino group of PEG has been replaced by an activated linker (see, for example, Karasiewicz et al., U.S. Pat. No. 5,382,657).

PEGylation by acylation typically requires reacting an active ester derivative of PEG with an IL-28 or IL-29 polypeptide. An example of an activated PEG ester is PEG esterified to N-hydroxysuccinimide As used herein, the term “acylation” includes the following types of linkages between IL-28 or IL-29 and a water-soluble polymer: amide, carbamate, urethane, and the like. Methods for preparing PEGylated IL-28 or IL-29 by acylation will typically comprise the steps of (a) reacting an IL-28 or IL-29 polypeptide with PEG (such as a reactive ester of an aldehyde derivative of PEG) under conditions whereby one or more PEG groups attach to IL-28 or IL-29, and (b) obtaining the reaction product(s). Generally, the optimal reaction conditions for acylation reactions will be determined based upon known parameters and desired results. For example, the larger the ratio of PEG:IL-28 or IL-29, the greater the percentage of polyPEGylated IL-28 or IL-29 product.

PEGylation by alkylation generally involves reacting a terminal aldehyde, e.g., propionaldehyde, butyraldehyde, acetaldehyde, and the like, derivative of PEG with IL-28 or IL-29 in the presence of a reducing agent. PEG groups are preferably attached to the polypeptide via a —CH₂—NH₂ group.

Derivatization via reductive alkylation to produce a monoPEGylated product takes advantage of the differential reactivity of different types of primary amino groups available for derivatization. Typically, the reaction is performed at a pH that allows one to take advantage of the pKa differences between the ε-amino groups of the lysine residues and the α-amino group of the N-terminal residue of the protein. By such selective derivatization, attachment of a water-soluble polymer that contains a reactive group such as an aldehyde, to a protein is controlled. The conjugation with the polymer occurs predominantly at the N-terminus of the protein without significant modification of other reactive groups such as the lysine side chain amino groups.

Reductive alkylation to produce a substantially homogenous population of monopolymer IL-28 or IL-29 conjugate molecule can comprise the steps of: (a) reacting an IL-28 or IL-29 polypeptide with a reactive PEG under reductive alkylation conditions at a pH suitable to permit selective modification of the α-amino group at the amino terminus of the IL-28 or IL-29, and (b) obtaining the reaction product(s). The reducing agent used for reductive alkylation should be stable in aqueous solution and preferably be able to reduce only the Schiff base formed in the initial process of reductive alkylation. Preferred reducing agents include sodium borohydride, sodium cyanoborohydride, dimethylamine borane, trimethylamine borane, and pyridine borane.

For a substantially homogenous population of monopolymer IL-28 or IL-29 conjugates, the reductive alkylation reaction conditions are those that permit the selective attachment of the water-soluble polymer moiety to the N-terminus of IL-28 or IL-29. Such reaction conditions generally provide for pKa differences between the lysine amino groups and the α-amino group at the N-terminus. The pH also affects the ratio of polymer to protein to be used. In general, if the pH is lower, a larger excess of polymer to protein will be desired because the less reactive the N-terminal α-group, the more polymer is needed to achieve optimal conditions. If the pH is higher, the polymer: IL-28 or IL-29 need not be as large because more reactive groups are available. Typically, the pH will fall within the range of 3-9, or 3-6. Another factor to consider is the molecular weight of the water-soluble polymer. Generally, the higher the molecular weight of the polymer, the fewer number of polymer molecules which may be attached to the protein. For PEGylation reactions, the typical molecular weight is about 2 kDa to about 100 kDa, about 5 kDa to about 50 kDa, or about 12 kDa to about 40 kDa. The molar ratio of water-soluble polymer to IL-28 or IL-29 will generally be in the range of 1:1 to 100:1. Typically, the molar ratio of water-soluble polymer to IL-28 or IL-29 will be 1:1 to 20:1 for polyPEGylation, and 1:1 to 5:1 for monoPEGylation.

General methods for producing conjugates comprising interferon and water-soluble polymer moieties are known in the art. See, for example, Karasiewicz et al., U.S. Pat. No. 5,382,657, Greenwald et al., U.S. Pat. No. 5,738,846, Nieforth et al., Clin. Pharmacol. Ther. 59:636 (1996), Monkarsh et al., Anal. Biochem. 247:434 (1997). PEGylated species can be separated from unconjugated IL-28 or IL-29 polypeptides using standard purification methods, such as dialysis, ultrafiltration, ion exchange chromatography, affinity chromatography, size exclusion chromatography, and the like.

The IL-28 or IL-29 polypeptides of the present invention are capable of specifically binding the IL-28 receptor and/or acting as an antiviral agent. The binding of IL-28 or 11-29 polypeptides to the IL-28 receptor can be assayed using established approaches. IL-28 or IL-29 polypeptides can be iodinated using an iodobead (Pierce, Rockford, Ill.) according to manufacturer's directions, and the ¹²⁵I-IL-28 or ¹²⁵I-IL-29 can then be used as described below.

In a first approach fifty nanograms of ¹²⁵I-IL-28 or ¹²⁵I-IL-29 can be combined with 1000 ng of IL-28 receptor human IgG fusion protein, in the presence or absence of possible binding competitors including unlabeled cysteine mutant IL-28, cysteine mutant IL-29, IL-28, or IL-29. The same binding reactions would also be performed substituting other cytokine receptor human IgG fusions as controls for specificity. Following incubation at 4° C., protein-G (Zymed, San Francisco, Calif.) is added to the reaction, to capture the receptor-IgG fusions and any proteins bound to them, and the reactions are incubated another hour at 4° C. The protein-G sepharose is then collected, washed three times with PBS and ¹²⁵I-IL-28 or ¹²⁵I-IL-29 bound is measure by gamma counter (Packard Instruments, Downers Grove, Ill.).

In a second approach, the ability of molecules to inhibit the binding of ¹²⁵I-IL-28 or ¹²⁵I-IL-29 to plate bound receptors can be assayed. A fragment of the IL-28 receptor, representing the extracellular, ligand binding domain, can be adsorbed to the wells of a 96 well plate by incubating 100 μl of 1 g/mL solution of receptor in the plate overnight. In a second form, a receptor-human IgG fusion can be bound to the wells of a 96 well plate that has been coated with an antibody directed against the human IgG portion of the fusion protein. Following coating of the plate with receptor the plate is washed, blocked with SUPERBLOCK (Pierce, Rockford, Ill.) and washed again. Solutions containing a fixed concentration of ¹²⁵I-IL-28 or ¹²⁵I-IL-29 with or without increasing concentrations of potential binding competitors including, Cysteine mutant IL-28, cysteine mutant IL-29, IL-28 and IL-29, and 100 μl of the solution added to appropriate wells of the plate. Following a one hour incubation at 4° C. the plate is washed and the amount ¹²⁵I-IL-28 or ¹²⁵I-IL-29 bound determined by counting (Topcount, Packard Instruments, Downers grove, IL). The specificity of binding of ¹²⁵I-IL-28 or ¹²⁵I-IL-29 can be defined by receptor molecules used in these binding assays as well as by the molecules used as inhibitors.

In addition to pegylation, human albumin can be coupled to an IL-28 or IL-29 polypeptide of the present invention to prolong its half-life. Human albumin is the most prevalent naturally occurring blood protein in the human circulatory system, persisting in circulation in the body for over twenty days. Research has shown that therapeutic proteins genetically fused to human albumin have longer half-lives. An IL28 or IL29 albumin fusion protein, like pegylation, may provide patients with long-acting treatment options that offer a more convenient administration schedule, with similar or improved efficacy and safety compared to currently available treatments (U.S. Pat. No. 6,165,470; Syed et al., Blood, 89(9):3243-3253 (1997); Yeh et al., Proc. Natl. Acad. Sci. USA, 89:1904-1908 (1992); and Zeisel et al., Horm. Res., 37:5-13 (1992)).

Like the aforementioned peglyation and human albumin, an Fc portion of the human IgG molecule can be fused to a polypeptide of the present invention. The resultant fusion protein may have an increased circulating half-life due to the Fc moiety (U.S. Pat. No. 5,750,375, U.S. Pat. No. 5,843,725, U.S. Pat. No. 6,291,646; Barouch et al., Journal of Immunology, 61:1875-1882 (1998); Barouch et al., Proc. Natl. Acad. Sci. USA, 97(8):4192-4197 (Apr. 11, 2000); and Kim et al., Transplant Proc., 30(8):4031-4036 (December 1998)).

IL-28A, IL-29, IL-28B, zcyto24 and zcyto25, each have been shown to form a complex with the orphan receptor designated zcytor19 (IL-28RA). IL-28RA is described in a commonly assigned patent application PCT/US01/44808. IL-28B, IL-29, zcyto24, and zcyto25 have been shown to bind or signal through IL-28RA as well, further supporting that IL-28A, IL-29, IL-28B, zcyto24 and zcyto25 are members of the same family of cytokines. IL-28RA receptor is a class II cytokine receptor. Class II cytokine receptors usually bind to four-helix-bundle cytokines. For example, interleukin-10 and the interferons bind receptors in this class (e.g., interferon-gamma receptor, alpha and beta chains and the interferon-alpha/beta receptor alpha and beta chains).

Class II cytokine receptors are characterized by the presence of one or more cytokine receptor modules (CRM) in their extracellular domains. Other class II cytokine receptors include zcytor11 (commonly owned U.S. Pat. No. 5,965,704), CRF2-4 (Genbank Accession No. Z17227), IL-10R (Genbank Accession Nos. U00672 and NM_{—}001558), DIRS1, zcytor7 (commonly owned U.S. Pat. No. 5,945,511), and tissue factor. IL-28RA, like all known class II receptors except interferon-alpha/beta receptor alpha chain, has only a single class II CRM in its extracellular domain.

Analysis of a human cDNA clone encoding IL-28RA (SEQ ID NO:11) revealed an open reading frame encoding 520 amino acids (SEQ ID NO:12) comprising a secretory signal sequence (residues 1 (Met) to 20 (Gly) of SEQ ID NO:12) and a mature IL-28RA cytokine receptor polypeptide (residues 21 (Arg) to 520 (Arg) of SEQ ID NO:12) an extracellular ligand-binding domain of approximately 206 amino acid residues (residues 21 (Arg) to 226 (Asn) of SEQ ID NO:12), a transmembrane domain of approximately 23 amino acid residues (residues 227 (Trp) to 249 (Trp) of SEQ ID NO:12), and an intracellular domain of approximately 271 amino acid residues (residues 250 (Lys) to 520 (Arg) of SEQ ID NO:12). Within the extracellular ligand-binding domain, there are two fibronectin type III domains and a linker region. The first fibronectin type III domain comprises residues 21 (Arg) to 119 (Tyr) of SEQ ID NO:12, the linker comprises residues 120 (Leu) to 124 (Glu) of SEQ ID NO:12, and the second fibronectin type III domain comprises residues 125 (Pro) to 223 (Pro) of SEQ ID NO:12.

In addition, a human cDNA clone encoding a IL-28RA variant with a 29 amino acid deletion was identified. This IL-28RA variant (as shown in SEQ ID NO:13) comprises an open reading frame encoding 491 amino acids (SEQ ID NO:14) comprising a secretory signal sequence (residues 1 (Met) to 20 (Gly) of SEQ ID NO:14) and a mature IL-28RA cytokine receptor polypeptide (residues 21 (Arg) to 491 (Arg) of SEQ ID NO:14) an extracellular ligand-binding domain of approximately 206 amino acid residues (residues 21 (Arg) to 226 (Asn) of SEQ ID NO:14, a transmembrane domain of approximately 23 amino acid residues (residues 227 (Trp) to 249 (Trp) of SEQ ID NO:14), and an intracellular domain of approximately 242 amino acid residues (residues 250 (Lys) to 491 (Arg) of SEQ ID NO:14).

A truncated soluble form of the IL-28RA receptor mRNA appears to be naturally expressed. Analysis of a human cDNA clone encoding the truncated soluble IL-28RA (SEQ ID NO:15) revealed an open reading frame encoding 211 amino acids (SEQ ID NO:16) comprising a secretory signal sequence (residues 1 (Met) to 20 (Gly) of SEQ ID NO:16) and a mature truncated soluble IL-28RA receptor polypeptide (residues 21 (Arg) to 211 (Ser) of SEQ ID NO:16) a truncated extracellular ligand-binding domain of approximately 143 amino acid residues (residues 21 (Arg) to 163 (Trp) of SEQ ID NO:16), no transmembrane domain, but an additional domain of approximately 48 amino acid residues (residues 164 (Lys) to 211 (Ser) of SEQ ID NO:16).

IL-28RA is a member of the same receptor subfamily as the class II cytokine receptors, and receptors in this subfamily may associate to form homodimers that transduce a signal. Several members of the subfamily (e.g., receptors that bind interferon, IL-10, IL-19, and IL-TIF) combine with a second subunit (termed a β-subunit) to bind ligand and transduce a signal. However, in many cases, specific β-subunits associate with a plurality of specific cytokine receptor subunits. For example, class II cytokine receptors, such as, zcytor11 (U.S. Pat. No. 5,965,704) and CRF2-4 receptor heterodimerize to bind the cytokine IL-TIF (See, WIPO publication WO 00/24758; Dumontier et al., J. Immunol. 164:1814-1819, 2000; Spencer, S D et al., J. Exp. Med. 187:571-578, 1998; Gibbs, V C and Pennica Gene 186:97-101, 1997 (CRF2-4 cDNA); Xie, M H et al., J. Biol. Chem. 275: 31335-31339, 2000). IL-10β receptor is believed to be synonymous with CRF2-4 (Dumoutier, L. et al., Proc. Nat'l. Acad. Sci. 97:10144-10149, 2000; Liu Y et al, J Immunol 152; 1821-1829, 1994 (IL-10R cDNA). Therefore, one could expect that IL-28, IL-29, zcyto24 and zcyto25 would bind either monomeric, homodimeric, heterodimeric and multimeric zcytor19 receptors. Experimental evidence has identified CRF2-4 as the putative binding partner for IL-28RA.

Examples of biological activity for molecules used to identify IL-28 or IL-29 molecules that are useful in the methods of the present invention include molecules that can bind to the IL-28 receptor with some specificity. Generally, a ligand binding to its cognate receptor is specific when the K_{D} falls within the range of 100 nM to 100 pM. Specific binding in the range of 100 mM to 10 nM K_{D} is low affinity binding. Specific binding in the range of 2.5 pM to 100 pM K_{D} is high affinity binding. In another example, biologically active IL-28 or IL-29 molecules are capable of some level of antiviral activity associated with wildtype IL-28 or IL-29.

The various codons that encode for a given amino acid are set forth below in Table 2.

TABLE 2

| | One | | Degen- |
|---|---|---|---|
| Amino | Letter | | erate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by referencing the sequences disclosed herein. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid Threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Sequences containing preferential codons can be tested and optimized for expression in various species, and tested for functionality as disclosed herein.

As previously noted, the isolated polynucleotides of the present invention include DNA and RNA. Methods for preparing DNA and RNA are well known in the art. In general, RNA is isolated from a tissue or cell that produces large amounts of IL-28 or IL-29 RNA. Such tissues and cells are identified by Northern blotting (Thomas, Proc. Natl. Acad. Sci. USA 77:5201, 1980), or by screening conditioned medium from various cell types for activity on target cells or tissue. Once the activity or RNA producing cell or tissue is identified, total RNA can be prepared using guanidinium isothiocyanate extraction followed by isolation by centrifugation in a CsCl gradient (Chirgwin et al., Biochemistry 18:52-94, 1979). Poly (A)⁺RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding IL-28 or IL-29 polypeptides are then identified and isolated by, for example, hybridization or PCR.

A full-length clones encoding IL-28 or IL-29 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to IL-28 receptor fragments, or other specific binding partners.

Those skilled in the art will recognize that the sequence disclosed in, for example, SEQ ID NOs:17, 19 and 21, represent a single allele of human IL-28A, IL-29, and IL28B, respectively, and that allelic variation and alternative splicing are expected to occur. For example, an IL-29 variant has been identified where amino acid residue 169 as shown in SEQ ID NO:19 is an Asn residue whereas its corresponding amino acid residue in SEQ ID NO:4 is an Arg residue, as described in WO 02/086087. Such allelic variants are included in the present invention. Allelic variants of IL-28 and IL-29 molecules of the present invention can be cloned by probing cDNA or genomic libraries from different individuals according to standard procedures. Allelic variants of the DNA sequence shown in SEQ ID NOs:17, 19, and 21, including those containing silent mutations and those in which mutations result in amino acid sequence changes, in addition to the cysteine mutations, are within the scope of the present invention, as are proteins which are allelic variants of SEQ ID NO:18, 20, and 22. cDNAs generated from alternatively spliced mRNAs, which retain the properties of IL-28 or IL-29 polypeptides, are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art, and mutations to the polynucleotides encoding cysteines or cysteine residues can be introduced as described herein.

Within embodiments of the invention, isolated IL-28 and IL-29-encoding nucleic acid molecules can hybridize under stringent conditions to nucleic acid molecules having the nucleotide sequence selected from the group of SEQ ID NOs:1, 3, 5, 17, 19, 21, 23, 25, 27, 29, 31, 33, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 109, 111, 113, 115, 117, 119, 121, 123, 125, 127, 129, 131, 133, 135, 137, 139, 141, 143, 145, 147, 149, or to its complement thereof. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe.

A pair of nucleic acid molecules, such as DNA-DNA, RNA-RNA and DNA-RNA, can hybridize if the nucleotide sequences have some degree of complementarity. Hybrids can tolerate mismatched base pairs in the double helix, but the stability of the hybrid is influenced by the degree of mismatch. The Tₘ of the mismatched hybrid decreases by 1° C. for every 1-1.5% base pair mismatch. Varying the stringency of the hybridization conditions allows control over the degree of mismatch that will be present in the hybrid. The degree of stringency increases as the hybridization temperature increases and the ionic strength of the hybridization buffer decreases.

It is well within the abilities of one skilled in the art to adapt these conditions for use with a particular polynucleotide hybrid. The Tₘ for a specific target sequence is the temperature (under defined conditions) at which 50% of the target sequence will hybridize to a perfectly matched probe sequence. Those conditions which influence the Tₘ include, the size and base pair content of the polynucleotide probe, the ionic strength of the hybridization solution, and the presence of destabilizing agents in the hybridization solution. Numerous equations for calculating Tₘ are known in the art, and are specific for DNA, RNA and DNA-RNA hybrids and polynucleotide probe sequences of varying length (see, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user defined criteria. Such programs can also analyze a given sequence under defined conditions and identify suitable probe sequences. Typically, hybridization of longer polynucleotide sequences, >50 base pairs, is performed at temperatures of about 20-25° C. below the calculated Tₘ. For smaller probes, <50 base pairs, hybridization is typically carried out at the Tₘ or 5-10° C. below the calculated Tₘ. This allows for the maximum rate of hybridization for DNA-DNA and DNA-RNA hybrids.

Following hybridization, the nucleic acid molecules can be washed to remove non-hybridized nucleic acid molecules under stringent conditions, or under highly stringent conditions. Typical stringent washing conditions include washing in a solution of 0.5×-2×SSC with 0.1% sodium dodecyl sulfate (SDS) at 55-65° C. That is, nucleic acid molecules encoding an IL-28 or IL-29 polypeptide hybridize with a nucleic acid molecule having the nucleotide sequence selected from the group of SEQ ID NOs:1, 3, 5, 17, 19, 21, 23, 25, 27, 29, 31, 33, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 109, 111, 113, 115, 117, 119, 121, 123, 125, 127, 129, 131, 133, 135, 137, 139, 141, 143, 145, 147, 149 or its complement thereof, under stringent washing conditions, in which the wash stringency is equivalent to 0.5×-2×SSC with 0.1% SDS at 55-65° C., including 0.5×SSC with 0.1% SDS at 55° C., or 2×SSC with 0.1% SDS at 65° C. One of skill in the art can readily devise equivalent conditions, for example, by substituting SSPE for SSC in the wash solution.

Typical highly stringent washing conditions include washing in a solution of 0.1×-0.2×SSC with 0.1% sodium dodecyl sulfate (SDS) at 50-65° C. In other words, nucleic acid molecules encoding a variant of an IL-28 or IL-29 polypeptide hybridize with a nucleic acid molecule having the nucleotide sequence selected from the group of SEQ ID NOs:1, 3, 5, 17, 19, 21, 23, 25, 27, 29, 31, 33, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 109, 111, 113, 115, 117, 119, 121, 123, 125, 127, 129, 131, 133, 135, 137, 139, 141, 143, 145, 147, 149, or its complement thereof, under highly stringent washing conditions, in which the wash stringency is equivalent to 0.1×-0.2×SSC with 0.1% SDS at 50-65° C., including 0.1×SSC with 0.1% SDS at 50° C., or 0.2×SSC with 0.1% SDS at 65° C.

The present invention also provides isolated IL-28 or IL-29 polypeptides that have a substantially similar sequence identity to the polypeptides of the present invention, for example, selected from the group of SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and 150. The term “substantially similar sequence identity” is used herein to denote polypeptides comprising at least 80%, at least 90%, at least 95%, at least 96%, at least 97%, at least 97.5%, at least 98%, at least 98.5%, at least 99%, at least 99.5%, or greater than 99.5% sequence identity to the amino acid sequences selected from the group of SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and 150. The present invention also includes polypeptides that comprise an amino acid sequence having at least 80%, at least 90%, at least 95%, at least 96%, at least 97%, at least 97.5%, at least 98%, at least 98.5%, at least 99%, at least 99.5%, or greater than 99.5% sequence identity to a polypeptide or fragment thereof of the present invention. The present invention further includes nucleic acid molecules that encode such polypeptides. The IL-28 and IL-29 polypeptides of the present invention are preferably recombinant polypeptides. In another aspect, the IL-28 and IL-29 polypeptides of the present invention have at least 15, at least 30, at least 45, or at least 60 sequential amino acids. For example, an IL-29 polypeptide of the present invention relates to a polypeptide having at least 15, at least 30, at least 45, or at least 60 sequential amino acids to an amino acid sequence selected from the group of SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and 150. Methods for determining percent identity are herein.

The present invention also contemplates variant nucleic acid molecules that can be identified using two criteria: a determination of the similarity between the encoded polypeptide with the amino acid sequence selected from the group of SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and 150 and/or a hybridization assay, as described above. Such variants include nucleic acid molecules: (1) that hybridize with a nucleic acid molecule having the nucleotide sequence selected from the group of SEQ ID NOs:1, 3, 5, 17, 19, 21, 23, 25, 27, 29, 31, 33, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 109, 111, 113, 115, 117, 119, 121, 123, 125, 127, 129, 131, 133, 135, 137, 139, 141, 143, 145, 147, 149, or complement thereof, under stringent washing conditions, in which the wash stringency is equivalent to 0.5×-2×SSC with 0.1% SDS at 55-65° C.; or (2) that encode a polypeptide having at least 80%, at least 90%, at least 95%, at least 96%, at least 97%, at least 97.5%, at least 98%, at least 98.5%, at least 99%, at least 99.5%, or greater than 99.5% sequence identity to the amino acid sequence selected from the group of SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and 150. Alternatively, variants can be characterized as nucleic acid molecules: (1) that hybridize with a nucleic acid molecule having the nucleotide sequence selected from the group of SEQ ID NOs:1, 3, 5, 17, 19, 21, 23, 25, 27, 29, 31, 33, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 109, 111, 113, 115, 117, 119, 121, 123, 125, 127, 129, 131, 133, 135, 137, 139, 141, 143, 145, 147, 149 or its complement thereof, under highly stringent washing conditions, in which the wash stringency is equivalent to 0.1×-0.2×SSC with 0.1% SDS at 50-65° C.; and (2) that encode a polypeptide having at least 80%, at least 90%, at least 95%, at least 96%, at least 97%, at least 98%, at least 99%, at least 99.5%, or greater than 99.5% sequence identity to the amino acid sequence selected from the group of SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and 150.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.) as shown in Table 2 (amino acids are indicated by the standard one-letter codes).

$$\frac{Total number of identical matches}{[ \begin{matrix}length of the longer sequence plus the number of \\ gaps introduced into the longer sequence \\ in order to align the two sequences\end{matrix} ]} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant IL-28 or IL-29. The FASTA algorithm is described by Pearson and Lipman, Proc. Nat'l Acad. Sci. USA 85:2444 (1988), and by Pearson, Meth. Enzmmol. 183:63 (1990).

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzmmol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

IL-28 or IL-29 polypeptides with substantially similar sequence identity are characterized as having one or more amino acid substitutions, deletions or additions. These changes are preferably of a minor nature, that is conservative amino acid substitutions (see Table 4) and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. The present invention thus includes polypeptides that comprise a sequence that is at least 80%, at least 90%, at least 95%, at least 96%, at least 97%, at least 97.5%, at least 98%, at least 98.5%, at least 99%, at least 99.5%, or greater than 99.5% identical to the corresponding region of SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and 150. Polypeptides comprising affinity tags can further comprise a proteolytic cleavage site between the IL-28 and IL-29 polypeptide and the affinity tag. Preferred such sites include thrombin cleavage sites and factor Xa cleavage sites.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

Determination of amino acid residues that comprise regions or domains that are critical to maintaining structural integrity can be determined. Within these regions one can determine specific residues that will be more or less tolerant of change and maintain the overall tertiary structure of the molecule. Methods for analyzing sequence structure include, but are not limited to alignment of multiple sequences with high amino acid or nucleotide identity, secondary structure propensities, binary patterns, complementary packing and buried polar interactions (Barton, Current Opin. Struct. Biol. 5:372-376, 1995 and Cordes et al., Current Opin. Struct. Biol. 6:3-10, 1996). In general, when designing modifications to molecules or identifying specific fragments determination of structure will be accompanied by evaluating activity of modified molecules.

Amino acid sequence changes are made in IL-28 or IL-29 polypeptides so as to minimize disruption of higher order structure essential to biological activity. For example, where the IL-28 or IL-29 polypeptide comprises one or more helices, changes in amino acid residues will be made so as not to disrupt the helix geometry and other components of the molecule where changes in conformation abate some critical function, for example, binding of the molecule to its binding partners. The effects of amino acid sequence changes can be predicted by, for example, computer modeling as disclosed above or determined by analysis of crystal structure (see, e.g., Lapthorn et al., Nat. Struct. Biol. 2:266-268, 1995). Other techniques that are well known in the art compare folding of a variant protein to a standard molecule (e.g., the native protein). For example, comparison of the cysteine pattern in a variant and standard molecules can be made. Mass spectrometry and chemical modification using reduction and alkylation provide methods for determining cysteine residues which are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). It is generally believed that if a modified molecule does not have the same cysteine pattern as the standard molecule folding would be affected. Another well known and accepted method for measuring folding is circular dichrosism (CD). Measuring and comparing the CD spectra generated by a modified molecule and standard molecule is routine (Johnson, Proteins 7:205-214, 1990). Crystallography is another well known method for analyzing folding and structure. Nuclear magnetic resonance (NMR), digestive peptide mapping and epitope mapping are also known methods for analyzing folding and structurally similarities between proteins and polypeptides (Schaanan et al., Science 257:961-964, 1992).

A Hopp/Woods hydrophilicity profile of an IL-28 or IL-29 polypeptide sequence selected from the group of SEQ ID NOs:2, 4, 6, 18, 20, 22, 24, 26, 28, 30, 32, 34, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 112, 114, 116, 118, 120, 122, 124, 126, 128, 130, 132, 134, 136, 138, 140, 142, 144, 146, 148 and 150 can be generated (Hopp et al., Proc. Natl. Acad. Sci. 78:3824-3828, 1981; Hopp, J. Immun. Meth. 88:1-18, 1986 and Triquier et al., Protein Engineering 11:153-169, 1998). The profile is based on a sliding six-residue window. Buried G, S, and T residues and exposed H, Y, and W residues were ignored. Those skilled in the art will recognize that hydrophilicity or hydrophobicity will be taken into account when designing modifications in the amino acid sequence of an IL-28 or IL-29 polypeptide, so as not to disrupt the overall structural and biological profile. Of particular interest for replacement are hydrophobic residues selected from the group consisting of Val, Leu and Ile or the group consisting of Met, Gly, Ser, Ala, Tyr and Trp.

The identities of essential amino acids can also be inferred from analysis of sequence similarity between IFN-α and members of the family of IL-28A, IL-28B, and IL-29 (as shown in Tables 1 and 2). Using methods such as “FASTA” analysis described previously, regions of high similarity are identified within a family of proteins and used to analyze amino acid sequence for conserved regions. An alternative approach to identifying a variant polynucleotide on the basis of structure is to determine whether a nucleic acid molecule encoding a potential variant IL-28 or IL-29 gene can hybridize to a nucleic acid molecule as discussed above.

Other methods of identifying essential amino acids in the polypeptides of the present invention are procedures known in the art, such as site-directed mutagenesis or alanine-scanning mutagenesis (Cunningham and Wells, Science 244:1081 (1989), Bass et al., Proc. Natl. Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant IL-28 and IL-29 molecules are tested for biological or biochemical activity as disclosed below to identify amino acid residues that are critical to the activity of the molecule. See also, Hilton et al., J. Biol. Chem. 271:4699 (1996).

The present invention also includes functional fragments of IL-28 or IL-29 polypeptides and nucleic acid molecules encoding such functional fragments. A “functional” IL-28 or IL-29 or fragment thereof as defined herein is characterized by its proliferative or differentiating activity, by its ability to induce or inhibit specialized cell functions, or by its ability to bind specifically to an anti-IL-28 or IL-29 antibody or IL-28 receptor (either soluble or immobilized). The specialized activities of IL-28 or IL-29 polypeptides and how to test for them are disclosed herein. As previously described herein, IL-28 and IL-29 polypeptides are characterized by a six-helical-bundle. Thus, the present invention further provides fusion proteins encompassing: (a) polypeptide molecules comprising one or more of the helices described above; and (b) functional fragments comprising one or more of these helices. The other polypeptide portion of the fusion protein may be contributed by another helical-bundle cytokine or interferon, such as IFN-α, or by a non-native and/or an unrelated secretory signal peptide that facilitates secretion of the fusion protein.

The IL-28 or IL-29 polypeptides of the present invention, including full-length polypeptides, biologically active fragments, and fusion polypeptides can be produced according to conventional techniques using cells into which have been introduced an expression vector encoding the polypeptide. As used herein, “cells into which have been introduced an expression vector” include both cells that have been directly manipulated by the introduction of exogenous DNA molecules and progeny thereof that contain the introduced DNA. Suitable host cells are those cell types that can be transformed or transfected with exogenous DNA and grown in culture, and include bacteria, fungal cells, and cultured higher eukaryotic cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding an IL-28 or IL-29 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct an IL-28 or IL-29 polypeptide into the secretory pathway of a host cell, a secretory signal sequence (also known as a leader sequence, prepro sequence or pre sequence) is provided in the expression vector. The secretory signal sequence may be that of IL-28 or IL-29, e.g., SEQ ID NO:119 or SEQ ID NO:121, or may be derived from another secreted protein (e.g., t-PA; see, U.S. Pat. No. 5,641,655) or synthesized de novo. The secretory signal sequence is operably linked to an IL-28 or IL-29 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

A wide variety of suitable recombinant host cells includes, but is not limited to, gram-negative prokaryotic host organisms. Suitable strains of E. coli include W3110 and mutants-strains thereof (e.g, an OmpT protease deficient W3110 strain, and an OmpT protease and fhuA deficient W3110 strain), K12-derived strains MM294, TG-1, JM-107, BL21, and UT5600. Other suitable strains include: BL21(DE3), BL21(DE3)pLysS, BL21(DE3)pLysE, DH1, DH4I, DH5, DH5I, DH51F′, DH5IMCR, DH10B, DH10B/p3, DH11S, C600, HB101, JM101, JM105, JM109, JM110, K38, RR1, Y1088, Y1089, CSH18, ER1451, ER1647, E. coli K12, E. coli K12 RV308, E. coli K12 C600, E. coli HB101, E. coli K12 C600 R.sub.k-M.sub.k-, E. coli K12 RR1 (see, for example, Brown (ed.), Molecular Biology Labfax (Academic Press 1991)). Other gram-negative prokaryotic hosts can include Serratia, Pseudomonas, Caulobacter. Prokaryotic hosts can include gram-positive organisms such as Bacillus, for example, B. subtilis and B. thuringienesis, and B. thuringienesis var. israelensis, as well as Streptomyces, for example, S. lividans, S. ambofaciens, S. fradiae, and S. griseofuscus. Suitable strains of Bacillus subtilus include BR151, YB886, MI119, MI120, and B170 (see, for example, Hardy, “Bacillus Cloning Methods,” in DNA Cloning: A Practical Approach, Glover (ed.) (IRL Press 1985)). Standard techniques for propagating vectors in prokaryotic hosts are well-known to those of skill in the art (see, for example, Ausubel et al. (eds.), Short Protocols in Molecular Biology, 3^{rd} Edition (John Wiley & Sons 1995); Wu et al., Methods in Gene Biotechnology (CRC Press, Inc. 1997)). In one embodiment, the methods of the present invention use Cysteine mutant IL-28 or IL-29 expressed in the W3110 strain, which has been deposited at the American Type Culture Collection (ATCC) as ATCC #27325.

When large scale production of an IL-28 or IL-29 polypeptide using the expression system of the present invention is required, batch fermentation can be used. Generally, batch fermentation comprises that a first stage seed flask is prepared by growing E. coli strains expressing an IL-28 or IL-29 polypeptide in a suitable medium in shake flask culture to allow for growth to an optical density (OD) of between 5 and 20 at 600 nm. A suitable medium would contain nitrogen from a source(s) such as ammonium sulfate, ammonium phosphate, ammonium chloride, yeast extract, hydrolyzed animal proteins, hydrolyzed plant proteins or hydrolyzed caseins. Phosphate will be supplied from potassium phosphate, ammonium phosphate, phosphoric acid or sodium phosphate. Other components would be magnesium chloride or magnesium sulfate, ferrous sulfate or ferrous chloride, and other trace elements. Growth medium can be supplemented with carbohydrates, such as fructose, glucose, galactose, lactose, and glycerol, to improve growth. Alternatively, a fed batch culture is used to generate a high yield of IL-28 or IL-29 polypeptide. The IL-28 or IL-29 polypeptide producing E. coli strains are grown under conditions similar to those described for the first stage vessel used to inoculate a batch fermentation.

Following fermentation the cells are harvested by centrifugation, re-suspended in homogenization buffer and homogenized, for example, in an APV-Gaulin homogenizer (Invensys APV, Tonawanda, N.Y.) or other type of cell disruption equipment, such as bead mills or sonicators. Alternatively, the cells are taken directly from the fermentor and homogenized in an APV-Gaulin homogenizer. The washed inclusion body prep can be solubilized using guanidine hydrochloride (5-8 M) or urea (7-8 M) containing a reducing agent such as beta mercaptoethanol (10-100 mM) or dithiothreitol (5-50 mM). The solutions can be prepared in Tris, phosphate, HEPES or other appropriate buffers. Inclusion bodies can also be solubilized with urea (2-4 M) containing sodium lauryl sulfate (0.1-2%). In the process for recovering purified IL-28 or IL-29 from transformed E. coli host strains in which the IL-28 or IL-29 is accumulates as retractile inclusion bodies, the cells are disrupted and the inclusion bodies are recovered by centrifugation. The inclusion bodies are then solubilized and denatured in 6 M guanidine hydrochloride containing a reducing agent. The reduced IL-28 or IL-29 is then oxidized in a controlled renaturation step. Refolded IL-28 or IL-29 can be passed through a filter for clarification and removal of insoluble protein. The solution is then passed through a filter for clarification and removal of insoluble protein. After the IL-28 or IL-29 protein is refolded and concentrated, the refolded IL-28 or IL-29 protein is captured in dilute buffer on a cation exchange column and purified using hydrophobic interaction chromatography.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Manassas, Va. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. The second method of making recombinant baculovirus utilizes a transposon-based system described by Luckow (Luckow, V. A, et al., J Virol 67:4566-79, 1993). This system is sold in the Bac-to-Bac kit (Life Technologies, Rockville, Md.). This system utilizes a transfer vector, pFastBac1™ (Life Technologies) containing a Tn7 transposon to move the DNA encoding the Cysteine mutant IL-28 or IL-29 polypeptide into a baculovirus genome maintained in E. coli as a large plasmid called a “bacmid.” The pFastBac1™ transfer vector utilizes the AcNPV polyhedrin promoter to drive the expression of the gene of interest, in this case IL-28 or IL-29. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol. Chem. 270:1543-9, 1995. In such transfer vector constructs, a short or long version of the basic protein promoter can be used. Moreover, transfer vectors can be constructed which replace the native IL-28 or IL-29 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native IL-28 or IL-29 secretory signal sequence. In addition, transfer vectors can include an in-frame fusion with DNA encoding an epitope tag at the C- or N-terminus of the expressed Cysteine mutant IL-28 or IL-29 polypeptide, for example, a Glu-Glu epitope tag (Grussenmeyer, T. et al., Proc. Natl. Acad. Sci. 82:7952-4, 1985). Using techniques known in the art, a transfer vector containing IL-28 or IL-29 is transformed into E. Coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses IL-28 or IL-29 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435).

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. The use of Pichia methanolica as host for the production of recombinant proteins is disclosed in U.S. Pat. Nos. 5,955,349, 5,888,768 and 6,001,597, U.S. Pat. No. 5,965,389, U.S. Pat. No. 5,736,383, and U.S. Pat. No. 5,854,039.

It is preferred to purify the polypeptides and proteins of the present invention to ≧80% purity, more preferably to ≧90% purity, even more preferably ≧95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide or protein is substantially free of other polypeptides or proteins, particularly those of animal origin.

Expressed recombinant IL-28 or IL-29 proteins (including chimeric polypeptides and multimeric proteins) are purified by conventional protein purification methods, typically by a combination of chromatographic techniques. See, in general, Affinity Chromatography: Principles & Methods, Pharmacia LKB Biotechnology, Uppsala, Sweden, 1988; and Scopes, Protein Purification: Principles and Practice, Springer-Verlag, New York, 1994. Proteins comprising a polyhistidine affinity tag (typically about 6 histidine residues) are purified by affinity chromatography on a nickel chelate resin. See, for example, Houchuli et al., Bio/Technol. 6: 1321-1325, 1988. Proteins comprising a glu-glu tag can be purified by immunoaffinity chromatography according to conventional procedures. See, for example, Grussenmeyer et al., supra. Maltose binding protein fusions are purified on an amylose column according to methods known in the art.

IL-28 or IL-29 polypeptides can also be prepared through chemical synthesis according to methods known in the art, including exclusive solid phase synthesis, partial solid phase methods, fragment condensation or classical solution synthesis. See, for example, Merrifield, J. Am. Chem. Soc. 85:2149, 1963; Stewart et al., Solid Phase Peptide Synthesis (2nd edition), Pierce Chemical Co., Rockford, Ill., 1984; Bayer and Rapp, Chem. Pept. Prot. 3:3, 1986; and Atherton et al., Solid Phase Peptide Synthesis: A Practical Approach, IRL Press, Oxford, 1989. In vitro synthesis is particularly advantageous for the preparation of smaller polypeptides.

Generally, the dosage of administered IL-28 or IL29 polypeptide of the present invention will vary depending upon such factors as the patient's age, weight, height, sex, general medical condition and previous medical history. Typically, it is desirable to provide the recipient with a dosage of IL-28 or IL29 polypeptide which is in the range of from about 1 pg/kg to 10 mg/kg (amount of agent/body weight of patient), although a lower or higher dosage also may be administered as circumstances dictate. One skilled in the art can readily determine such dosages, and adjustments thereto, using methods known in the art.

Administration of an IL-28 or IL29 polypeptide to a subject can be topical, inhalant, intravenous, intraarterial, intraperitoneal, intramuscular, subcutaneous, intrapleural, intrathecal, by perfusion through a regional catheter, or by direct intralesional injection. When administering therapeutic proteins by injection, the administration may be by continuous infusion or by single or multiple boluses.

Additional routes of administration include oral, mucosal-membrane, pulmonary, and transcutaneous. Oral delivery is suitable for polyester microspheres, zein microspheres, proteinoid microspheres, polycyanoacrylate microspheres, and lipid-based systems (see, for example, DiBase and Morrel, “Oral Delivery of Microencapsulated Proteins,” in Protein Delivery: Physical Systems, Sanders and Hendren (eds.), pages 255-288 (Plenum Press 1997)). The feasibility of an intranasal delivery is exemplified by such a mode of insulin administration (see, for example, Hinchcliffe and Illum, Adv. Drug Deliv. Rev. 35:199 (1999)). Dry or liquid particles comprising IL-28 or IL29 polypeptide can be prepared and inhaled with the aid of dry-powder dispersers, liquid aerosol generators, or nebulizers (e.g., Pettit and Gombotz, TIBTECH 16:343 (1998); Patton et al., Adv. Drug Deliv. Rev. 35:235 (1999)). This approach is illustrated by the AERX diabetes management system, which is a hand-held electronic inhaler that delivers aerosolized insulin into the lungs. Studies have shown that proteins as large as 48,000 kDa have been delivered across skin at therapeutic concentrations with the aid of low-frequency ultrasound, which illustrates the feasibility of trascutaneous administration (Mitragotri et al., Science 269:850 (1995)). Transdermal delivery using electroporation provides another means to administer a molecule having IL-28 or IL29 polypeptide activity (Potts et al., Pharm. Biotechnol. 10:213 (1997)).

A pharmaceutical composition comprising a protein, polypeptide, or peptide having IL-28 or IL29 polypeptide activity can be formulated according to known methods to prepare pharmaceutically useful compositions, whereby the therapeutic proteins are combined in a mixture with a pharmaceutically acceptable vehicle. A composition is said to be in a “pharmaceutically acceptable vehicle” if its administration can be tolerated by a recipient patient. Sterile phosphate-buffered saline is one example of a pharmaceutically acceptable vehicle. Other suitable vehicles are well-known to those in the art. See, for example, Gennaro (ed.), Remington's Pharmaceutical Sciences, 19th Edition (Mack Publishing Company 1995).

For purposes of therapy, molecules having IL-28 or IL29 polypeptide activity and a pharmaceutically acceptable vehicle are administered to a patient in a therapeutically effective amount. A combination of a protein, polypeptide, or peptide having IL-28 or IL29 polypeptide activity and a pharmaceutically acceptable vehicle is said to be administered in a “therapeutically effective amount” or “effective amount” if the amount administered is physiologically significant. An agent is physiologically significant if its presence results in a detectable change in the physiology of a recipient patient. For example, an agent used to treat inflammation is physiologically significant if its presence alleviates at least a portion of the inflammatory response.

A pharmaceutical composition comprising IL-28 or IL29 polypeptide of the present invention can be furnished in liquid form, in an aerosol, or in solid form. Liquid forms, are illustrated by injectable solutions, aerosols, droplets, topological solutions and oral suspensions. Exemplary solid forms include capsules, tablets, and controlled-release forms. The latter form is illustrated by miniosmotic pumps and implants (Bremer et al., Pharm. Biotechnol. 10:239 (1997); Ranade, “Implants in Drug Delivery,” in Drug Delivery Systems, Ranade and Hollinger (eds.), pages 95-123 (CRC Press 1995); Bremer et al., “Protein Delivery with Infusion Pumps,” in Protein Delivery: Physical Systems, Sanders and Hendren (eds.), pages 239-254 (Plenum Press 1997); Yewey et al., “Delivery of Proteins from a Controlled Release Injectable Implant,” in Protein Delivery: Physical Systems, Sanders and Hendren (eds.), pages 93-117 (Plenum Press 1997)). Other solid forms include creams, pastes, other topological applications, and the like.

Liposomes provide one means to deliver therapeutic polypeptides to a subject intravenously, intraperitoneally, intrathecally, intramuscularly, subcutaneously, or via oral administration, inhalation, or intranasal administration. Liposomes are microscopic vesicles that consist of one or more lipid bilayers surrounding aqueous compartments (see, generally, Bakker-Woudenberg et al., Eur. J. Clin. Microbiol. Infect. Dis. 12 (Suppl. 1):S61 (1993), Kim, Drugs 46:618 (1993), and Ranade, “Site-Specific Drug Delivery Using Liposomes as Carriers,” in Drug Delivery Systems, Ranade and Hollinger (eds.), pages 3-24 (CRC Press 1995)). Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 μm to greater than 10 μm. A variety of agents can be encapsulated in liposomes: hydrophobic agents partition in the bilayers and hydrophilic agents partition within the inner aqueous space(s) (see, for example, Machy et al., Liposomes In Cell Biology And Pharmacology (John Libbey 1987), and Ostro et al., American J. Hosp. Pharm. 46:1576 (1989)). Moreover, it is possible to control the therapeutic availability of the encapsulated agent by varying liposome size, the number of bilayers, lipid composition, as well as the charge and surface characteristics of the liposomes.

Liposomes can adsorb to virtually any type of cell and then slowly release the encapsulated agent. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446:368 (1985)). After intravenous administration, small liposomes (0.1 to 1.0 μm) are typically taken up by cells of the reticuloendothelial system, located principally in the liver and spleen, whereas liposomes larger than 3.0 μm are deposited in the lung. This preferential uptake of smaller liposomes by the cells of the reticuloendothelial system has been used to deliver chemotherapeutic agents to macrophages and to tumors of the liver.

The reticuloendothelial system can be circumvented by several methods including saturation with large doses of liposome particles, or selective macrophage inactivation by pharmacological means (Claassen et al., Biochim. Biophys. Acta 802:428 (1984)). In addition, incorporation of glycolipid- or polyethelene glycol-derivatized phospholipids into liposome membranes has been shown to result in a significantly reduced uptake by the reticuloendothelial system (Allen et al., Biochim. Biophys. Acta 1068:133 (1991); Allen et al., Biochim. Biophys. Acta 1150:9 (1993)).

Liposomes can also be prepared to target particular cells or organs by varying phospholipid composition or by inserting receptors or ligands into the liposomes. For example, liposomes, prepared with a high content of a nonionic surfactant, have been used to target the liver (Hayakawa et al., Japanese Patent 04-244,018; Kato et al., Biol. Pharm. Bull. 16:960 (1993)). These formulations were prepared by mixing soybean phospatidylcholine, α-tocopherol, and ethoxylated hydrogenated castor oil (HCO-60) in methanol, concentrating the mixture under vacuum, and then reconstituting the mixture with water. A liposomal formulation of dipalmitoylphosphatidylcholine (DPPC) with a soybean-derived sterylglucoside mixture (SG) and cholesterol (Ch) has also been shown to target the liver (Shimizu et al., Biol. Pharm. Bull. 20:881 (1997)).

Alternatively, various targeting ligands can be bound to the surface of the liposome, such as antibodies, antibody fragments, carbohydrates, vitamins, and transport proteins. For example, liposomes can be modified with branched type galactosyllipid derivatives to target asialoglycoprotein (galactose) receptors, which are exclusively expressed on the surface of liver cells (Kato and Sugiyama, Crit. Rev. Ther. Drug Carrier Syst. 14:287 (1997); Murahashi et al., Biol. Pharm. Bull. 20:259 (1997)). Similarly, Wu et al., Hepatology 27:772 (1998), have shown that labeling liposomes with asialofetuin led to a shortened liposome plasma half-life and greatly enhanced uptake of asialofetuin-labeled liposome by hepatocytes. On the other hand, hepatic accumulation of liposomes comprising branched type galactosyllipid derivatives can be inhibited by preinjection of asialofetuin (Murahashi et al., Biol. Pharm. Bull. 20:259 (1997)). Polyaconitylated human serum albumin liposomes provide another approach for targeting liposomes to liver cells (Kamps et al., Proc. Nat'l Acad. Sci. USA 94:11681 (1997)). Moreover, Geho, et al. U.S. Pat. No. 4,603,044, describe a hepatocyte-directed liposome vesicle delivery system, which has specificity for hepatobiliary receptors associated with the specialized metabolic cells of the liver.

In a more general approach to tissue targeting, target cells are prelabeled with biotinylated antibodies specific for a ligand expressed by the target cell (Harasym et al., Adv. Drug Deliv. Rev. 32:99 (1998)). After plasma elimination of free antibody, streptavidin-conjugated liposomes are administered. In another approach, targeting antibodies are directly attached to liposomes (Harasym et al., Adv. Drug Deliv. Rev. 32:99 (1998)).

Polypeptides having IL-28 or IL29 polypeptide activity can be encapsulated within liposomes using standard techniques of protein microencapsulation (see, for example, Anderson et al., Infect. Immun. 31:1099 (1981), Anderson et al., Cancer Res. 50:1853 (1990), and Cohen et al., Biochim. Biophys. Acta 1063:95 (1991), Alving et al. “Preparation and Use of Liposomes in Immunological Studies,” in Liposome Technology, 2nd Edition, Vol. III, Gregoriadis (ed.), page 317 (CRC Press 1993), Wassef et al., Meth. Enzymol. 149:124 (1987)). As noted above, therapeutically useful liposomes may contain a variety of components. For example, liposomes may comprise lipid derivatives of poly(ethylene glycol) (Allen et al., Biochim. Biophys. Acta 1150:9 (1993)).

Degradable polymer microspheres have been designed to maintain high systemic levels of therapeutic proteins. Microspheres are prepared from degradable polymers such as poly(lactide-co-glycolide) (PLG), polyanhydrides, poly (ortho esters), nonbiodegradable ethylvinyl acetate polymers, in which proteins are entrapped in the polymer (Gombotz and Pettit, Bioconjugate Chem. 6:332 (1995); Ranade, “Role of Polymers in Drug Delivery,” in Drug Delivery Systems, Ranade and Hollinger (eds.), pages 51-93 (CRC Press 1995); Roskos and Maskiewicz, “Degradable Controlled Release Systems Useful for Protein Delivery,” in Protein Delivery: Physical Systems, Sanders and Hendren (eds.), pages 45-92 (Plenum Press 1997); Bartus et al., Science 281:1161 (1998); Putney and Burke, Nature Biotechnology 16:153 (1998); Putney, Curr. Opin. Chem. Biol. 2:548 (1998)). Polyethylene glycol (PEG)-coated nanospheres can also provide vehicles for intravenous administration of therapeutic proteins (see, for example, Gref et al., Pharm. Biotechnol. 10:167 (1997)).

Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington Pharmaceutical Sciences, 19^{th} Edition (Mack Publishing Company 1995), and by Ranade and Hollinger, Drug Delivery Systems (CRC Press 1996).

As an illustration, pharmaceutical compositions may be supplied as a kit comprising a container that comprises an IL-28 or IL29 polypeptide of the present invention. Therapeutic polypeptides can be provided in the form of an injectable solution for single or multiple doses, or as a sterile powder that will be reconstituted before injection. Alternatively, such a kit can include a dry-powder disperser, liquid aerosol generator, or nebulizer for administration of a therapeutic polypeptide. Such a kit may further comprise written information on indications and usage of the pharmaceutical composition. Moreover, such information may include a statement that the IL-28 or IL29 polypeptide composition is contraindicated in patients with known hypersensitivity to IL-28 or IL29 polypeptide. The kit may further comprise at least one additional antiviral agent selected from the group of Interferon alpha, Interferon beta, Interferon gamma, Interferon omega, protease inhibitor, RNA or DNA polymerase inhibitor, nucleoside analog, antisense inhibitor, and combinations thereof. The additional antiviral agent included in the kit, for example, can be RIBAVIRIN™, PEG-INTRON®, PEGASYS®, or a combination thereof. It can also be advantageous for patients with a viral infection, such as hepatitis C, to take their medicine consistently and get the appropriate dose for their individualized therapy. Thus, a kit may optionally also include a small needle, with a self-priming feature and a large, easy-to-read dosing knob. This will help patients feel confident that they are getting an accurate dose and offers an easy-to-use alternative for people who may be intimidated by a traditional needle and syringe system. For example, the kit may include a disposable, one-time use precision dosing system that allows patients to administer an IL-28 or IL-29 molecule of the present invention in three easy steps: Mix, Dial and Deliver. (1) Mixing occurs by simply pushing down on the pen to combine the IL-28 or IL-29 molecule powder with sterile water, both of which are stored in the body of the pen; (2) Dialing allows patients to accurately select their predetermined individualized dose; and (3) Delivery allows patients to inject their individualized dose of the medication (See, for example, Schering Plough's PEG-INTRON REDIPEN).

IL-28 and IL-29 polypeptides of the present invention can be used in treating, ablating, curing, preventing, inhibiting, reducing, or delaying onset of liver specific diseases, in particular liver disease where viral infection is in part an etiologic agent. In particular IL-28 and IL-29 polypeptides will be used to treat a mammal with a viral infection selected from the group consisting of hepatitis A, hepatitis B, hepatitis C, and hepatitis D. When liver disease is inflammatory and continuing for at least six months, it is generally considered chronic hepatitis. Hepatitis C virus (HCV) patients actively infected will be positive for HCV-RNA in their blood, which is detectable by reverse transcriptase/polymerase chain reaction (RT-PCR) assays. The methods of the present invention will slow the progression of the liver disease. Clinically, diagnostic tests for HCV include serologic assays for antibodies and molecular tests for viral particles. Enzyme immunoassays are available (Vrielink et al., Transfusion 37:845-849, 1997), but may require confirmation using additional tests such as an immunoblot assay (Pawlotsky et al., Hepatology 27:1700-1702, 1998). Qualitative and quantitative assays generally use polymerase chain reaction techniques, and are preferred for assessing viremia and treatment response (Poynard et al., Lancet 352:1426-1432, 1998; McHutchinson et al., N. Engl. J. Med. 339:1485-1492, 1998). Several commercial tests are available, such as, quantitative RT-PCR (Amplicor HCV Monitor™, Roche Molecular Systems, Branchburg, N.J.) and a branched DNA (deoxyribonucleic acid) signal amplification assay (Quantiplex™ HCV RNA Assay [bDNA], Chiron Corp., Emeryville, Calif.). A non-specific laboratory test for liver inflammation or necrosis measures alanine aminotransferase level (ALT) and is inexpensive and readily available (National Institutes of Health Consensus Development Conference Panel, Hepatology 26 (Suppl. 1):2S-10S, 1997). Histologic evaluation of liver biopsy is generally considered the most accurate means for determining hepatitis progression (Yano et al., Hepatology 23:1334-1340, 1996.) For a review of clinical tests for HCV, see, Lauer et al., N. Engl. J. Med. 345:41-52, 2001.

There are several in vivo models for testing HBV and HCV that are known to those skilled in art. For example, the effects of IL-28 or IL-29 on mammals infected with HBV can be accessed using a woodchuck model. Briefly, woodchucks chronically infected with woodchuck hepatitis virus (WHV) develop hepatitis and hepatocellular carcinoma that is similar to disease in humans chronically infected with HBV. The model has been used for the preclinical assessment of antiviral activity. A chronically infected WHV strain has been established and neonates are inoculated with serum to provide animals for studying the effects of certain compounds using this model. (For a review, see, Tannant et al., ILAR J. 42 (2):89-102, 2001). Chimpanzees may also be used to evaluate the effect of IL-28 and IL-29 on HBV infected mammals. Using chimpanzees, characterization of HBV was made and these studies demonstrated that the chimpanzee disease was remarkably similar to the disease in humans (Barker et al., J. Infect. Dis. 132:451-458, 1975 and Tabor et al., J. Infect. Dis. 147:531-534, 1983.) The chimpanzee model has been used in evaluating vaccines (Prince et al., In: Vaccines 97 Cold Spring Harbor Laboratory Press, 1997.) Therapies for HIV are routinely tested using non-human primates infected with simian immunodeficiency viruses (for a review, see, Hirsch et al., Adv. Pharmcol. 49:437-477, 2000 and Nathanson et al., AIDS 13 (suppl. A):5113-5120, 1999.) For a review of use of non-human primates in HIV, hepatitis, malaria, respiratory syncytial virus, and other diseases, see, Sibal et al., ILAR J. 42 (2):74-84, 2001.

Other examples of the types of viral infections for which an IL-28 or IL-29 molecule of the present invention can be used in treating, ablating, curing, preventing, inhibiting, reducing, or delaying onset of viral symptoms include, but are not limited to: infections caused by DNA Viruses (e.g., Herpes Viruses such as Herpes Simplex viruses, Epstein-Barr virus, Cytomegalovirus; Pox viruses such as Variola (small pox) virus; Hepadnaviruses (e.g, Hepatitis B virus); Papilloma viruses; Adenoviruses); RNA Viruses (e.g., HIV I, II; HTLV I, II; Poliovirus; Hepatitis A; Orthomyxoviruses (e.g., Influenza viruses, e.g., avian influenza A virus, for instance the H5N1 virus); Paramyxoviruses (e.g., Measles virus); Rabies virus; Hepatitis C); Coronavirus (causes Severe Acute Respiratory Syndrome (SARS)); Rhinovirus, Respiratory Syncytial Virus, Norovirus, West Nile Virus, Yellow Fever, Rift Valley Virus, Lassa Fever Virus, Ebola Virus, Lymphocytic Choriomeningitis Virus, which replicates in tissues including liver, and the like. Moreover, examples of the types of diseases for which IL-28 and IL-29 could be used include, but are not limited to: Acquired immunodeficiency; Hepatitis; Gastroenteritis; Hemorrhagic diseases; Enteritis; Carditis; Encephalitis; Paralysis; Brochiolitis; Upper and lower respiratory disease; Respiratory Papillomatosis; Arthritis; Disseminated disease, hepatocellular carcinoma resulting from chronic Hepatitis C infection. In addition, viral disease in other tissues may be treated with IL-28A, IL-28B, and IL-29, for example viral meningitis, and HIV-related disease. For example, a transgenic model for testing the activity of a therapeutic sample is described in the following examples and described in Money, et al., Antiviral Ther., 3 (Suppl 3):59-68, 1998.

Animal models that are used to test for efficacy in specific viruses are known. For example, Dengue Virus can be tested using a model as such as described in Huang et al., J. Gen. Virol. September; 81(Pt 9):2177-82, 2000. West Nile Virus can be tested using the model as described in Xiao et al., Emerg. Infect. Dis. July-August; 7(4):714-21, 2001 or Mashimo et al., Proc. Natl. Acad. Sci. U S A. August 20; 99(17):11311-6, 2002. Venezuelan equine encephalitis virus model is described in Jackson et al., Veterinary Pathology, 28 (5): 410-418, 1991; Vogel et al., Arch. Pathol. Lab. Med. February; 120(2):164-72, 1996; Lukaszewski and Brooks, J. of Virology, 74(11):5006-5015, 2000. Rhinoviruses models are described in Yin and Lomax, J. Gen. Virol. 67 (Pt 11):2335-40, 1986. Models for respiratory syncytial virus are described in Byrd and Prince, Clin. Infect. Dis. 25(6):1363-8, 1997. Other models are known in the art and it is well within the skill of those ordinarily skilled in the art to know how to use such models.

Noroviruses (genus Norovirus, family Caliciviridae) are a group of related, single-stranded RNA, nonenveloped viruses that cause acute gastroenteritis in humans. Norovirus was recently approved as the official genus name for the group of viruses provisionally described as “Norwalk-like viruses” (NLV). Noroviruses are estimated to cause 23 million cases of acute gastroenteritis in the United States per year, and are the leading cause of gastroenteritis in the United States.

The symptoms of norovirus illness usually include nausea, vomiting, diarrhea, and some stomach cramping. Sometimes people additionally have a low-grade fever, chills, headache, muscle aches, and a general sense of tiredness. The illness often begins suddenly, and the infected person may feel very sick. The illness is usually brief, with symptoms lasting only about 1 or 2 days. In general, children experience more vomiting than adults. Most people with norovirus illness have both of these symptoms. Currently, there is no antiviral medication that works against norovirus and there is no vaccine to prevent infection.

Therapeutics to Noroviruses have been difficult to identify in part because of a lack of good cell culture systems and animal models of disease. The recent identification of a murine norovirus now allows testing of therapeutics such as IL-28 and IL-29 polypeptides of the present invention in a cell culture system (Wobus, Karst et al., “Replication of Norovirus in Cell Culture Reveals a Tropism for Dendritic Cells and Macrophages,” PLoS Biol, 2(12):e432, (2004)) and a mouse model of disease (Karst, Wobus et al., “STAT1-dependent innate immunity to a Norwalk-like virus,” Science, 299(5612):1575-8 (2003)).

Karst, S. M., C. E. Wobus, et al. (2003). “STAT1-dependent innate immunity to a Norwalk-like virus.” Science, 299(5612): 1575-8.

Norwalk-like caliciviruses (Noroviruses) cause over 90% of nonbacterial epidemic gastroenteritis worldwide, but the pathogenesis of norovirus infection is poorly understood because these viruses do not grow in cultured cells and there is no small animal model. Here, we report a previously unknown murine norovirus. Analysis of Murine Norovirus 1 infection revealed that signal transducer and activator of transcription 1-dependent innate immunity, but not T and B cell-dependent adaptive immunity, is essential for norovirus resistance. The identification of host molecules essential for murine norovirus resistance may provide targets for prevention or control of an important human disease.

Wobus, C. E., S. M. Karst, et al. (2004). “Replication of Norovirus in Cell Culture Reveals a Tropism for Dendritic Cells and Macrophages.” PLoS Biol, 2(12): e432.

Noroviruses are understudied because these important enteric pathogens have not been cultured to date. We found that the norovirus murine norovirus 1 (MNV-1) infects macrophage-like cells in vivo and replicates in cultured primary dendritic cells and macrophages. MNV-1 growth was inhibited by the interferon-alphabeta receptor and STAT-1, and was associated with extensive rearrangements of intracellular membranes. An amino acid substitution in the capsid protein of serially passaged MNV-1 was associated with virulence attenuation in vivo. This is the first report of replication of a norovirus in cell culture. The capacity of MNV-1 to replicate in a STAT-1-regulated fashion and the unexpected tropism of a norovirus for cells of the hematopoietic lineage provide important insights into norovirus biology.

IL-28 and IL-29 polypeptides of the present invention can be used in combination with antiviral agents, including those described above. Some of the more common treatments for viral infection include drugs that inhibit viral replication such as ACYCLOVIR™. In addition, the combined use of some of these agents form the basis for highly active antiretroviral therapy (HAART) used for the treatment of AIDS. Examples in which the combination of immunotherapy (i.e., cytokines) and antiviral drugs shows improved efficacy include the use of interferon plus RIBAVIRIN™ for the treatment of chronic hepatitis C infection (Maddrey, Semin. Liver. Dis. 19 Suppl 1:67-75, 1999) and the combined use of IL-2 and HAART (Ross, et al, ibid.) Thus, as IL-28 and IL-29 can stimulate the immune system against disease, it can similarly be used in HAART applications.

In particular, IL-28 and IL-29 polypeptides of the present invention may be useful in monotherapy or combination therapy with IFN-α, e.g., PEGASYS® or PEG-INTRON® (with or without a nucleoside analog, such as RIBAVIRIN™, lamivudine, entecavir, emtricitabine, telbivudine and tenofovir) or with a nucleoside analog, such as RIBAVIRIN™, lamivudine, entecavir, emtricitabine, telbivudine and tenofovir in patients who do not respond well to IFN therapy.

These patients may not respond to IFN therapy due to having less type I interferon receptor on the surface of their cells (Yatsuhashi H, et al., J Hepatol. Jun. 30(6):995-1003, 1999; Mathai et al., J Interferon Cytokine Res. Sep. 19(9):1011-8, 1999; Fukuda et al., J. Med. Virol. 63(3):220-7, 2001). IL-28A, IL-28B, and IL-29 may also be useful in monotherapy or combination therapy with IFN-α (with or without a nucleoside analog, such as RIBAVIRIN™, lamivudine, entecavir, emtricitabine, and telbivudine and tenofovir) or with a nucleoside analog, such as RIBAVIRIN™ in patients who have less type I interferon receptor on the surface of their cells due to down-regulation of the type I interferon receptor after type I interferon treatment (Dupont et al., J. Interferon Cytokine Res. 22 (4): 491-501, 2002).

IL-28 or IL-29 polypeptide may be used in combination with other immunotherapies including cytokines, immunoglobulin transfer, and various co-stimulatory molecules. In addition to antiviral drugs, IL-28 and IL-29 polypeptides of the present invention can be used in combination with any other immunotherapy that is intended to stimulate the immune system. Thus, IL-28 and IL-29 polypeptides could be used with other cytokines such as Interferon, IL-21, or IL-2. IL-28 and IL-29 can also be added to methods of passive immunization that involve immunoglobulin transfer, one example bring the use of antibodies to treat RSV infection in high risk patients (Meissner H C, ibid.). In addition, IL-28 and IL-29 polypeptides can be used with additional co-stimulatory molecules such as 4-1BB ligand that recognize various cell surface molecules like CD137 (Tan, J T et al., J Immunol 163:4859-68, 1999).

IL-28 and IL-29 can be used as a monotherapy for acute and chronic viral infections and for immunocompromised patients. Methods that enhance immunity can accelerate the recovery time in patients with unresolved infections. Immunotherapies can have an even greater impact on subsets of immunocompromised patients such as the very young or elderly as well as patients that suffer immunodeficiencies acquired through infection, or induced following medical interventions such as chemotherapy or bone marrow ablation. Examples of the types of indications being treated via immune-modulation include; the use of IFN-α for chronic hepatitis (Perry C M, and Jarvis B, Drugs 61:2263-88, 2001), the use of IL-2 following HIV infection (Mitsuyasu R., J. Infect. Dis. 185 Suppl 2:S115-22, 2002; and Ross R W et al., Expert Opin. Biol. Ther. 1:413-24, 2001), and the use of IFN-α (Faro A, Springer Semin. Immunopathol. 20:425-36, 1998) for treating Epstein Barr Virus infections following transplantation. Experiments performed in animal models indicate that IL-2 and GM-CSF may also be efficacious for treating EBV related diseases (Baiocchi R A et al., J Clin. Invest. 108:887-94, 2001).

IL-28 and IL-29 molecules of the present invention can be used as a monotherapy for acute and chronic viral infections and for immunocompromised patients. Methods that enhance immunity can accelerate the recovery time in patients with unresolved infections. In addition, IL-28 and IL-29 molecules of the present invention can be administered to a mammal in combination with other antiviral agents such as ACYCLOVIR™, RIBAVIRIN™, Interferons (e.g., PEGINTRON® and PEGASYS®), Serine Protease Inhibitors, Polymerase Inhibitors, Nucleoside Analogs, Antisense Inhibitors, and combinations thereof, to treat, ablate, cure, prevent, inhibit, reduce, or delay the onset of a viral infection selected from the group of hepatitis A, hepatitis B, hepatitis C, hepatitis D, respiratory syncytial virus, herpes virus, Epstein-Barr virus, influenza virus (e.g., avian influenza A virus, for instance the H5N1 virus), adenovirus, parainfluenza virus, Severe Acute Respiratory Syndrome, rhino virus, coxsackie virus, vaccinia virus, west nile virus, dengue virus, venezuelan equine encephalitis virus, pichinde virus, and polio virus. IL-28 and IL-29 polypeptides of the present invention can also be used in combination with other immunotherapies including cytokines, immunoglobulin transfer, and various co-stimulatory molecules. In addition, IL-28 and IL-29 molecules of the present invention can be used to treat a mammal with a chronic or acute viral infection that has resulted liver inflammation, thereby reducing the viral infection and/or liver inflammation. In particular IL-28 and IL-29 will be used to treat a mammal with a viral infection selected from the group of hepatitis A, hepatitis B, hepatitis C, and/or hepatitis D. IL-28 and IL-29 molecules of the present invention can also be used as an antiviral agent in viral infections selected from the group consisting of respiratory syncytial virus, herpes virus, Epstein-Barr virus, influenza virus (e.g., avian influenza A virus, for instance the H5N1 virus), adenovirus, parainfluenza virus, Severe Acute Respiratory Syndrome, rhino virus, coxsackie virus, vaccinia virus, west nile virus, dengue virus, venezuelan equine encephalitis virus, pichinde virus and polio virus.

The present invention is further illustrated by the following non-limiting examples.

### EXAMPLES

### Example 1

### Induction of IL-28A, IL-29 and IL-28B by Poly I:C and Viral Infection

Freshly isolated human peripheral blood mononuclear cells were grown in the presence of polyinosinic acid-polycytidylic acid (poly I:C; 100 mg/ml) (SIGMA; St. Louis, Mo.), encephalomyocarditis virus (EMCV) with an MOI of 0.1, or in medium alone. After a 15 h incubation, total RNA was isolated from cells and treated with RNase-free DNase. 100 ng total RNA was used as template for one-step RT-PCR using the Superscript One-Step RT-PCR with Platinum Taxi kit and gene-specific primers as suggested by the manufacturer (Invitrogen).

Low to undetectable amounts of human IL-28A, IL-28B, and IL-29, IFN-α and IFN-β RNA were seen in untreated cells. In contrast, the amount of IL-28A, IL-29, IL-28B RNA was increased by both poly I:C treatment and viral infection, as was also seen for the type I interferons. These experiments indicate that IL-28A, IL-29, IL-28B, like type I interferons, can be induced by double-stranded RNA or viral infection.

### Example 2

### IL-28 and IL-29 Signaling Activity Compared to IFNα in HepG2 Cells

### A. Cell Transfections

HepG2 cells were transfected as follows: 700,000 HepG2 cells/well (6 well plates) were plated approximately 18 h prior to transfection in 2 milliliters DMEM+10% fetal bovine serum. Per well, 1 microgram pISRE-Luciferase DNA (Stratagene) and 1 microgram pIRES2-EGFP DNA (Clontech,) were added to 6 microliters Fugene 6 reagent (Roche Biochemicals) in a total of 100 microliters DMEM. This transfection mix was added 30 minutes later to the pre-plated HepG2 cells. Twenty-four hours later the transfected cells were removed from the plate using trypsin-EDTA and replated at approximately 25,000 cells/well in 96 well microtiter plates. Approximately 18 h prior to ligand stimulation, media was changed to DMEM+0.5% FBS.

### B. Signal Transduction Reporter Assays

The signal transduction reporter assays were done as follows: Following an 18 h incubation at 37° C. in DMEM+0.5% FBS, transfected cells were stimulated with 100 ng/ml IL-28A, IL-29, IL-28B, zcyto24, zcyto25 and huIFN-α2a ligands. Following a 4-hour incubation at 37° degrees, the cells were lysed, and the relative light units (RLU) were measured on a luminometer after addition of a luciferase substrate. The results obtained are shown as the fold induction of the RLU of the experimental samples over the medium alone control (RLU of experimental samples/RLU of medium alone=fold induction). Table 5 shows that IL-28A, IL-29, IL-28B, zcyto24 and zcyto25 induce ISRE signaling in human HepG2 liver cells transfected with ISRE-luciferase.

TABLE 5
Fold Induction of Cytokine-dependent ISRE Signaling in HepG2 Cells

| Cytokine | Fold Induction |
|---|---|
| IL-28A | 5.6 |
| IL-29 | 4 |
| IL-28B | 5.8 |
| Zcyto24 | 4.7 |
| Zcyto25 | 3 |
| HuIFN-a2a | 5.8 |

### Example 3

### IL-29 Antiviral Activity Compared to IFNα in HepG2 Cells

An antiviral assay was adapted for EMCV (American Type Culture Collection # VR-129B, Manassas, Va.) with human cells (Familletti, P., et al., Methods Enzym. 78: 387-394, 1981). Cells were plated with cytokines and incubated 24 hours prior to challenge by EMCV at a multiplicity of infection of 0.1 to 1. The cells were analyzed for viability with a dye-uptake bioassay 24 hours after infection (Berg, K., et al., Apmis 98: 156-162, 1990). Target cells were given MTT and incubated at 37° C. for 2 hours. A solubiliser solution was added, incubated overnight at 37° C. and the optical density at 570 nm was determined OD570 is directly proportional to antiviral activity.

The results show the antiviral activity when IL-29 and IFN on were tested with HepG2 cells: IL-29, IFN-β and IFN α-2a were added at varying concentration to HepG2 cells prior to EMCV infection and dye-uptake assay. The mean and standard deviation of the OD570 from triplicate wells is plotted. OD570 is directly proportional to antiviral activity. For IL-29, the EC50 was 0.60 ng/ml; for IFN-α2a, the EC50 was 0.57 ng/ml; and for IFN-β, the EC50 was 0.46 ng/ml.

### Example 4

### IL-28RA mRNA Expression in Liver and Lymphocyte Subsets

In order to further examine the mRNA distribution for IL-28RA, semi-quantitative RT-PCR was performed using the SDS 7900HT system (Applied Biosystems, CA). One-step RT-PCR was performed using 100 ng total RNA for each sample and gene-specific primers. A standard curve was generated for each primer set using Bjab RNA and all sample values were normalized to HPRT. The normalized results are summarized in Tables 6-8. The normalized values for IFNAR2 and CRF2-4 are also shown.

Table 6: B and T cells express significant levels of IL-28RA mRNA. Low levels are seen in dendritic cells and most monocytes.

TABLE 6

| Cell/Tissue | IL-28RA | IFNAR2 | CRF2-4 |
|---|---|---|---|
| Dendritic Cells unstim | .04 | 5.9 | 9.8 |
| Dendritic Cells + IFNg | .07 | 3.6 | 4.3 |
| Dendritic Cells | .16 | 7.85 | 3.9 |
| CD14+ stim'd with LPS/IFNg | .13 | 12 | 27 |
| CD14+ monocytes resting | .12 | 11 | 15.4 |
| Hu CD14+ Unact. | 4.2 | TBD | TBD |
| Hu CD14+ 1 ug/ml LPS act. | 2.3 | TBD | TBD |
| H. Inflamed tonsil | 3 | 12.4 | 9.5 |
| H. B-cells + PMA/Iono 4 & 24 hrs | 3.6 | 1.3 | 1.4 |
| Hu CD19+ resting | 6.2 | TBD | TBD |
| Hu CD19+ 4 hr. PMA/Iono | 10.6 | TBD | TBD |
| Hu CD19+ 24 hr Act. PMA/Iono | 3.7 | TBD | TBD |
| IgD+ B-cells | 6.47 | 13.15 | 6.42 |
| IgM+ B-cells | 9.06 | 15.4 | 2.18 |
| IgD− B-cells | 5.66 | 2.86 | 6.76 |
| NKCells + PMA/Iono | 0 | 6.7 | 2.9 |
| Hu CD3+ Unactivated | 2.1 | TBD | TBD |
| CD4+ resting | .9 | 8.5 | 29.1 |
| CD4+ Unstim 18 hrs | 1.6 | 8.4 | 13.2 |
| CD4+ + Poly I/C | 2.2 | 4.5 | 5.1 |
| CD4+ + PMA/Iono | .3 | 1.8 | .9 |
| CD3 neg resting | 1.6 | 7.3 | 46 |
| CD3 neg unstim 18 hrs | 2.4 | 13.2 | 16.8 |
| CD3 neg + Poly I/C 18 hrs | 5.7 | 7 | 30.2 |
| CD3 neg + LPS 18 hrs | 3.1 | 11.9 | 28.2 |
| CD8+ unstim 18 hrs | 1.8 | 4.9 | 13.1 |
| CD8+ stim'd with PMA/Ion 18 hrs | .3 | .6 | 1.1 |

As shown in Table 7, normal liver tissue and liver derived cell lines display substantial levels of IL-28RA and CRF2-4 mRNA.

TABLE 7

| Cell/Tissue | IL-28RA | IFNAR2 | CRF2-4 |
|---|---|---|---|
| HepG2 | 1.6 | 3.56 | 2.1 |
| HepG2 UGAR 5/10/02 | 1.1 | 1.2 | 2.7 |
| HepG2, CGAT HKES081501C | 4.3 | 2.1 | 6 |
| HuH7 5/10/02 | 1.63 | 16 | 2 |
| HuH7 hepatoma - CGAT | 4.2 | 7.2 | 3.1 |
| Liver, normal - CGAT | 11.7 | 3.2 | 8.4 |
| #HXYZ020801K | | | |
| Liver, NAT - Normal adjacent tissue | 4.5 | 4.9 | 7.7 |
| Liver, NAT - Normal adjacent tissue | 2.2 | 6.3 | 10.4 |
| Hep SMVC hep vein | 0 | 1.4 | 6.5 |
| Hep SMCA hep. Artery | 0 | 2.1 | 7.5 |
| Hep. Fibro | 0 | 2.9 | 6.2 |
| Hep. Ca. | 3.8 | 2.9 | 5.8 |
| Adenoca liver | 8.3 | 4.2 | 10.5 |
| SK-Hep-1 adenoca. Liver | .1 | 1.3 | 2.5 |
| AsPC-1 Hu. Pancreatic adenocarc. | .7 | .8 | 1.3 |
| Hu. Hep. Stellate cells | .025 | 4.4 | 9.7 |

As shown in Table 8, primary airway epithelial cells contain abundant levels of IL-28RA and CRF2-4.

TABLE 8

| Cell/Tissue | IL-28RA | IFNAR2 | CRF2-4 |
|---|---|---|---|
| U87MG - glioma | 0 | .66 | .99 |
| NHBE unstim | 1.9 | 1.7 | 8.8 |
| NHBE + TNF-alpha | 2.2 | 5.7 | 4.6 |
| NHBE + poly I/C | 1.8 | nd | nd |
| Small Airway Epithelial Cells | 3.9 | 3.3 | 27.8 |
| NHLF - Normal human lung fibroblasts | 0 | nd | nd |

As shown in Table 8, IL-28RA is present in normal and diseased liver specimens, with increased expression in tissue from Hepatitis C and Hepatitis B infected specimens.

TABLE 8

| Cell/Tissue | IL-28RA | CRF2-4 | IFNAR2 |
|---|---|---|---|
| Liver with Coagulation Necrosis | 8.87 | 15.12 | 1.72 |
| Liver with Autoimmune Hepatitis | 6.46 | 8.90 | 3.07 |
| Neonatal Hepatitis | 6.29 | 12.46 | 6.16 |
| Endstage Liver disease | 4.79 | 17.05 | 10.58 |
| Fulminant Liver Failure | 1.90 | 14.20 | 7.69 |
| Fulminant Liver failure | 2.52 | 11.25 | 8.84 |
| Cirrhosis, primary biliary | 4.64 | 12.03 | 3.62 |
| Cirrhosis Alcoholic (Laennec's) | 4.17 | 8.30 | 4.14 |
| Cirrhosis, Cryptogenic | 4.84 | 7.13 | 5.06 |
| Hepatitis C+, with cirrhosis | 3.64 | 7.99 | 6.62 |
| Hepatitis C+ | 6.32 | 11.29 | 7.43 |
| Fulminant hepatitis secondary to Hep A | 8.94 | 21.63 | 8.48 |
| Hepatitis C+ | 7.69 | 15.88 | 8.05 |
| Hepatitis B+ | 1.61 | 12.79 | 6.93 |
| Normal Liver | 8.76 | 5.42 | 3.78 |
| Normal Liver | 1.46 | 4.13 | 4.83 |
| Liver NAT | 3.61 | 5.43 | 6.42 |
| Liver NAT | 1.97 | 10.37 | 6.31 |
| Hu Fetal Liver | 1.07 | 4.87 | 3.98 |
| Hepatocellular Carcinoma | 3.58 | 3.80 | 3.22 |
| Adenocarcinoma Liver | 8.30 | 10.48 | 4.17 |
| hep. SMVC, hep. Vein | 0.00 | 6.46 | 1.45 |
| Hep SMCA hep. Artery | 0.00 | 7.55 | 2.10 |
| Hep. Fibroblast | 0.00 | 6.20 | 2.94 |
| HuH7 hepatoma | 4.20 | 3.05 | 7.24 |
| HepG2 Hepatocellular carcinoma | 3.40 | 5.98 | 2.11 |
| SK-Hep-1 adenocar. Liver | 0.03 | 2.53 | 1.30 |
| HepG2 Unstim | 2.06 | 2.98 | 2.28 |
| HepG2 + zcyto21 | 2.28 | 3.01 | 2.53 |
| HepG2 + IFNα | 2.61 | 3.05 | 3.00 |
| Normal Female Liver - degraded | 1.38 | 6.45 | 4.57 |
| Normal Liver - degraded | 1.93 | 4.99 | 6.25 |
| Normal Liver - degraded | 2.41 | 2.32 | 2.75 |
| Disease Liver - degraded | 2.33 | 3.00 | 6.04 |
| Primary Hepatocytes from Clonetics | 9.13 | 7.97 | 13.30 |

As shown in Tables 9-13, IL-28RA is detectable in normal B cells, B lymphoma cell lines, T cells, T lymphoma cell lines (Jurkat), normal and transformed lymphocytes (B cells and T cells) and normal human monocytes.

TABLE 9

| | HPRT | IL-28RA | IL-28RA | | IFNR2 | | CRF2-4 |
|---|---|---|---|---|---|---|---|
| | Mean | Mean | norm | IFNAR2 | norm | CRF2-4 | Norm |
| CD14+ 24 hr unstim #A38 | 13.1 | 68.9 | 5.2 | 92.3 | 7.0 | 199.8 | 15.2 |
| CD14+ 24 hr stim #A38 | 6.9 | 7.6 | 1.1 | 219.5 | 31.8 | 276.6 | 40.1 |
| CD14+ 24 hr unstim #A112 | 17.5 | 40.6 | 2.3 | 163.8 | 9.4 | 239.7 | 13.7 |
| CD14+ 24 hr stim #A112 | 11.8 | 6.4 | 0.5 | 264.6 | 22.4 | 266.9 | 22.6 |
| CD14+ rest #X | 32.0 | 164.2 | 5.1 | 1279.7 | 39.9 | 699.9 | 21.8 |
| CD14+ + LPS #X | 21.4 | 40.8 | 1.9 | 338.2 | 15.8 | 518.0 | 24.2 |
| CD14+ 24 hr unstim #A39 | 26.3 | 86.8 | 3.3 | 297.4 | 11.3 | 480.6 | 18.3 |
| CD14+ 24 hr stim #A39 | 16.6 | 12.5 | 0.8 | 210.0 | 12.7 | 406.4 | 24.5 |
| HL60 Resting | 161.2 | 0.2 | 0.0 | 214.2 | 1.3 | 264.0 | 1.6 |
| HL60 + PMA | 23.6 | 2.8 | 0.1 | 372.5 | 15.8 | 397.5 | 16.8 |
| U937 Resting | 246.7 | 0.0 | 0.0 | 449.4 | 1.8 | 362.5 | 1.5 |
| U937 + PMA | 222.7 | 0.0 | 0.0 | 379.2 | 1.7 | 475.9 | 2.1 |
| Jurkat Resting | 241.7 | 103.0 | 0.4 | 327.7 | 1.4 | 36.1 | 0.1 |
| Jurkat Activated | 130.7 | 143.2 | 1.1 | | | | |
| Colo205 | 88.8 | 43.5 | 0.5 | | | | |
| HT-29 | 26.5 | 30.5 | 1.2 | | | | |

TABLE 10

| | HPRT SD | IL-28RA SD |
|---|---|---|
| Mono 24 hr unstim #A38 | 0.6 | 2.4 |
| Mono 24 hr stim #A38 | 0.7 | 0.2 |
| Mono 24 hr unstim | 2.0 | 0.7 |
| #A112 | | |
| Mono 24 hr stim #A112 | 0.3 | 0.1 |
| Mono rest #X | 5.7 | 2.2 |
| Mono + LPS #X | 0.5 | 1.0 |
| Mono 24 hr unstim #A39 | 0.7 | 0.8 |
| Mono 24 hr stim #A39 | 0.1 | 0.7 |
| HL60 Resting | 19.7 | 0.1 |
| HL60 + PMA | 0.7 | 0.4 |
| U937 Resting | 7.4 | 0.0 |
| U937 + PMA | 7.1 | 0.0 |
| Jurkat Resting | 3.7 | 1.1 |
| Jurkat Activated | 2.4 | 1.8 |
| Colo205 | 1.9 | 0.7 |
| HT-29 | 2.3 | 1.7 |

TABLE 11

| | Mean | Mean | Mean IL- | Mean |
|---|---|---|---|---|
| | Hprt | IFNAR2 | 28RA | CRF |
| CD3+/CD4+ 0 | 10.1 | 85.9 | 9.0 | 294.6 |
| CD4/CD3+ Unstim 18 hrs | 12.9 | 108.7 | 20.3 | 170.4 |
| CD4+/CD3+ + Poly I/C 18 hrs | 24.1 | 108.5 | 52.1 | 121.8 |
| CD4+/CD3+ + PMA/Iono 18 hrs | 47.8 | 83.7 | 16.5 | 40.8 |
| CD3 neg 0 | 15.4 | 111.7 | 24.8 | 706.1 |
| CD3 neg unstim 18 hrs | 15.7 | 206.6 | 37.5 | 263.0 |
| CD3 neg + Poly I/C 18 hrs | 9.6 | 67.0 | 54.7 | 289.5 |
| CD3 neg + LPS 18 hrs | 14.5 | 173.2 | 44.6 | 409.3 |
| CD8+ Unstim. 18 hrs | 6.1 | 29.7 | 11.1 | 79.9 |
| CD8+ + PMA/Iono 18 hrs | 78.4 | 47.6 | 26.1 | 85.5 |
| 12.8.1 - NHBE Unstim | 47.4 | 81.1 | 76.5 | 415.6 |
| 12.8.2 - NHBE + TNF-alpha | 42.3 | 238.8 | 127.7 | 193.9 |
| SAEC | 15.3 | 49.9 | 63.6 | 426.0 |

TABLE 12

| | IL-28RA | CRF | IFNAR2 | IL-28RA | CRF | IFNAR2 |
|---|---|---|---|---|---|---|
| | Norm | Norm | Norm | SD | SD | SD |
| CD3+/CD4+ 0 | 0.9 | 29.1 | 8.5 | 0.1 | 1.6 | 0.4 |
| CD4/CD3+ Unstim 18 hrs | 1.6 | 13.2 | 8.4 | 0.2 | 1.6 | 1.4 |
| CD4+/CD3+ + Poly I/C 18 hrs | 2.2 | 5.1 | 4.5 | 0.1 | 0.3 | 0.5 |
| CD4+/CD3+ + PMA/Iono 18 hrs | 0.3 | 0.9 | 1.8 | 0.0 | 0.1 | 0.3 |
| CD3 neg 0 | 1.6 | 46.0 | 7.3 | 0.2 | 4.7 | 1.3 |
| CD3 neg unstim 18 hrs | 2.4 | 16.8 | 13.2 | 0.4 | 2.7 | 2.3 |
| CD3 neg + Poly I/C 18 hrs | 5.7 | 30.2 | 7.0 | 0.3 | 1.7 | 0.8 |
| CD3 neg + LPS 18 hrs | 3.1 | 28.2 | 11.9 | 0.4 | 5.4 | 2.9 |
| CD8+ Unstim. 18 hrs | 1.8 | 13.1 | 4.9 | 0.1 | 1.1 | 0.3 |
| CD8+ + PMA/Iono 18 hrs | 0.3 | 1.1 | 0.6 | 0.0 | 0.1 | 0.0 |
| 12.8.1 - NHBE Unstim | 1.6 | 8.8 | 1.7 | 0.1 | 0.4 | 0.1 |
| 12.8.2 - NHBE + TNF-alpha | 3.0 | 4.6 | 5.7 | 0.1 | 0.1 | 0.1 |
| SAEC | 4.1 | 27.8 | 3.3 | 0.2 | 1.1 | 0.3 |

TABLE 13

| | SD | SD | SD IL- | SD |
|---|---|---|---|---|
| | Hprt | IFNAR2 | 28RA | CRF |
| CD3+/CD4+ 0 | 0.3 | 3.5 | 0.6 | 12.8 |
| CD4/CD3+ Unstim 18 hrs | 1.4 | 13.7 | 1.1 | 8.5 |
| CD4+/CD3+ + Poly I/C 18 hrs | 1.3 | 9.8 | 1.6 | 3.4 |
| CD4+/CD3+ + PMA/Iono 18 hrs | 4.0 | 10.3 | 0.7 | 3.7 |
| CD3 neg 0 | 1.4 | 16.6 | 1.6 | 28.6 |
| CD3 neg unstim 18 hrs | 2.4 | 16.2 | 2.7 | 12.6 |
| CD3 neg + Poly I/C 18 hrs | 0.5 | 7.0 | 1.0 | 8.3 |
| CD3 neg + LPS 18 hrs | 1.0 | 39.8 | 5.6 | 73.6 |
| CD8+ Unstim. 18 hrs | 0.2 | 1.6 | 0.5 | 6.1 |
| CD8+ + PMA/Iono 18 hrs | 1.3 | 1.7 | 0.2 | 8.1 |
| 12.8.1 - NHBE Unstim | 2.4 | 5.6 | 2.7 | 2.8 |
| 12.8.2 - NHBE + TNF-alpha | 0.5 | 3.4 | 3.5 | 3.4 |
| SAEC | 0.5 | 4.8 | 1.8 | 9.9 |

### Example 5

### Mouse IL-28 does not Effect Daudi Cell Proliferation

Human Daudi cells were suspended in RPMI+10% FBS at 50,000 cells/milliliter and 5000 cells were plated per well in a 96 well plate. IL-29-CEE (IL-29 conjugated with glu tag), IFN-γ or IFN-α2a was added in 2-fold serial dilutions to each well. IL-29-CEE was used at a concentration range of from 1000 ng/ml to 0.5 ng/ml. IFN-γ was used at a concentration range from 125 ng/ml to 0.06 ng/ml. IFN-α2a was used at a concentration range of from 62 ng/ml to 0.03 ng/ml. Cells were incubated for 72 h at 37° C. After 72 hours Alamar Blue (Accumed, Chicago, Ill.) was added at 20 microliters/well. Plates were further incubated at 37° C., 5% CO, for 24 hours. Plates were read on the Fmax™ plate reader (Molecular Devices, Sunnyvale, Calif.) using the SoftMax™ Pro program, at wavelengths 544 (Excitation) and 590 (Emission). Alamar Blue gives a fluourometric readout based on the metabolic activity of cells, and is thus a direct measurement of cell proliferation in comparison to a negative control. The results indicate that IL-29-CEE, in contrast to IFN-α2a, has no significant effect on proliferation of Daudi cells.

### Example 6

### Mouse IL-28 does not have Antiproliferative Effect on Mouse B Cells

Mouse B cells were isolated from 2 Balb/C spleens (7 months old) by depleting CD43+ cells using MACS magnetic beads. Purified B cells were cultured in vitro with LPS, anti-IgM or anti-CD40 monoclonal antibodies. Mouse IL-28 or mouse IFNα was added to the cultures and ³H-thymidine was added at 48 hrs. and ³H-thymidine incorporation was measured after 72 hrs. culture.

IFNα at 10 ng/ml inhibited ³H-thymidine incorporation by mouse B cells stimulated with either LPS or anti-IgM. However mouse IL-28 did not inhibit ³H-thymidine incorporation at any concentration tested including 1000 ng/ml. In contrast, both mIFNα and mouse IL-28 increased ³H thymidine incorporation by mouse B cells stimulated with anti-CD40 MAb.

These data demonstrate that mouse IL-28 unlike IFNα displays no antiproliferative activity even at high concentrations. In addition, zcyto24 enhances proliferation in the presence of anti-CD40 MAbs. The results illustrate that mouse IL-28 differs from IFNα in that mouse IL-28 does not display antiproliferative activity on mouse B cells, even at high concentrations. In addition, mouse IL-28 enhances proliferation in the presence of anti-CD40 monoclonal antibodies.

### Example 7

### Bone Marrow Expansion Assay

Fresh human marrow mononuclear cells (Poietic Technologies, Gaithersburg, Md.) were adhered to plastic for 2 hrs in αMEM, 10% FBS, 50 micromolar β-mercaptoethanol, 2 ng/ml FLT3L at 37° C. Non adherent cells were then plated at 25,000 to 45,000 cells/well (96 well tissue culture plates) in αMEM, 10% FBS, 50 micromolar β-mercaptoethanol, 2 ng/ml FLT3L in the presence or absence of 1000 ng/ml IL-29-CEE, 100 ng/ml IL-29-CEE, 10 ng/ml IL-29-CEE, 100 ng/ml IFN-α2a, 10 ng/ml IFN-α2a or 1 ng/ml IFN-α2a. These cells were incubated with a variety of cytokines to test for expansion or differentiation of hematopoietic cells from the marrow (20 ng/ml IL-2, 2 ng/ml IL-3, 20 ng/ml IL-4, 20 ng/ml IL-5, 20 ng/ml IL-7, 20 ng/ml IL-10, 20 ng/ml IL-12, 20 ng/ml IL-15, 10 ng/ml IL-21 or no added cytokine). After 8 to 12 days Alamar Blue (Accumed, Chicago, Ill.) was added at 20 microliters/well. Plates were further incubated at 37° C., 5% CO, for 24 hours. Plates were read on the Fmax™ plate reader (Molecular Devices Sunnyvale, Calif.) using the SoftMax™ Pro program, at wavelengths 544 (Excitation) and 590 (Emission). Alamar Blue gives a fluourometric readout based on the metabolic activity of cells, and is thus a direct measurement of cell proliferation in comparison to a negative control.

IFN-α2a caused a significant inhibition of bone marrow expansion under all conditions tested. In contrast, IL-29 had no significant effect on expansion of bone marrow cells in the presence of IL-3, IL-4, IL-5, IL-7, IL-10, IL-12, IL-21 or no added cytokine. A small inhibition of bone marrow cell expansion was seen in the presence of IL-2 or IL-15.

### Example 8

### Inhibition of IL-28 and IL-29 Signaling with Soluble Receptor (zcytoR19/CRF2-4)

### A. Signal Transduction Reporter Assay

A signal transduction reporter assay can be used to show the inhibitor properties of zcytor19-Fc4 homodimeric and zcytor19-Fc/CRF2-4-Fc heterodimeric soluble receptors on zcyto20, zcyto21 and zcyto24 signaling. Human embryonal kidney (HEK) cells overexpressing the zcytor19 receptor are transfected with a reporter plasmid containing an interferon-stimulated response element (ISRE) driving transcription of a luciferase reporter gene. Luciferase activity following stimulation of transfected cells with ligands (including zcyto20 (SEQ ID NO:18), zcyto21 (SEQ ID NO:20), zcyto24 (SEQ ID NO:8)) reflects the interaction of the ligand with soluble receptor.

### B. Cell Transfections

293 HEK cells overexpressing zcytor19 were transfected as follows: 700,000 293 cells/well (6 well plates) were plated approximately 18 h prior to transfection in 2 milliliters DMEM+10% fetal bovine serum. Per well, 1 microgram pISRE-Luciferase DNA (Stratagene) and 1 microgram pIRES2-EGFP DNA (Clontech,) were added to 6 microliters Fugene 6 reagent (Roche Biochemicals) in a total of 100 microliters DMEM. This transfection mix was added 30 minutes later to the pre-plated 293 cells. Twenty-four hours later the transfected cells were removed from the plate using trypsin-EDTA and replated at approximately 25,000 cells/well in 96 well microtiter plates. Approximately 18 h prior to ligand stimulation, media was changed to DMEM+0.5% FBS.

### C. Signal Transduction Reporter Assays

The signal transduction reporter assays were done as follows: Following an 18 h incubation at 37° C. in DMEM+0.5% FBS, transfected cells were stimulated with 10 ng/ml zcyto20, zcyto21 or zcyto24 and 10 micrograms/ml of the following soluble receptors; human zcytor19-Fc homodimer, human zcytor19-Fc/human CRF2-4-Fc heterodimer, human CRF2-4-Fc homodimer, murine zcytor19-Ig homodimer. Following a 4-hour incubation at 37° C., the cells were lysed, and the relative light units (RLU) were measured on a luminometer after addition of a luciferase substrate. The results obtained are shown as the percent inhibition of ligand-induced signaling in the presence of soluble receptor relative to the signaling in the presence of PBS alone. Table 13 shows that the human zcytor19-Fc/human CRF2-4 heterodimeric soluble receptor is able to inhibit zcyto20, zcyto21 and zcyto24-induced signaling between 16 and 45% of control. The human zcytor19-Fc homodimeric soluble receptor is also able to inhibit zcyto21-induced signaling by 45%. No significant effects were seen with huCRF2-4-Fc or muzcytor19-Ig homodimeric soluble receptors.

TABLE 14
Percent Inhibition of Ligand-induced Interferon Stimulated Response
Element (ISRE) Signaling by Soluble Receptors

| | Huzcytor19- | Huzcytor19- | HuCRF2-4- | |
|---|---|---|---|---|
| Ligand | Fc/huCRF2-4-Fc | Fc | Fc | Muzcytor19-Ig |
| Zcyto20 | 16% | 92% | 80% | 91% |
| Zcyto21 | 16% | 45% | 79% | 103% |
| Zcyto24 | 47% | 90% | 82% | 89% |

### Example 9

### IL-28 and IL-29 Inhibit HIV Replication in Fresh Human PBMCs

Human immunodeficiency virus (HIV) is a pathogenic retrovirus that infects cells of the immune system. CD4 T cells and monocytes are the primary infected cell types. To test the ability of IL-28 and IL-29 to inhibit HIV replication in vitro, PBMCs from normal donors were infected with the HIV virus in the presence of IL-28, IL-29 and MetIL-29C172S-PEG.

Fresh human peripheral blood mononuclear cells (PBMCs) were isolated from whole blood obtained from screened donors who were seronegative for HIV and HBV. Peripheral blood cells were pelleted/washed 2-3 times by low speed centrifugation and resuspended in PBS to remove contaminating platelets. The washed blood cells were diluted 1:1 with Dulbecco's phosphate buffered saline (D-PBS) and layered over 14 mL of Lymphocyte Separation Medium ((LSM; Cellgro™ by Mediatech, Inc. Herndon, Va.); density 1.078+/−0.002 g/ml) in a 50 mL centrifuge tube and centrifuged for 30 minutes at 600×G. Banded PBMCs were gently aspirated from the resulting interface and subsequently washed 2× in PBS by low speed centrifugation. After the final wash, cells were counted by trypan blue exclusion and resuspended at 1×10⁷ cells/mL in RPMI 1640 supplemented with 15% Fetal Bovine Serum (FBS), 2 mM L-glutamine, 4 μg/mL PHA-P. The cells were allowed to incubate for 48-72 hours at 37° C. After incubation, PBMCs were centrifuged and resuspended in RPMI 1640 with 15% FBS, 2 mM L-glutamine, 100 U/mL penicillin, 100 μg/mL streptomycin, 10 μg/mL gentamycin, and 20 U/mL recombinant human IL-2. PBMCs were maintained in the medium at a concentration of 1-2×10⁶ cells/mL with biweekly medium changes until used in the assay protocol. Monocytes were depleted from the culture as the result of adherence to the tissue culture flask.

For the standard PBMC assay, PHA-P stimulated cells from at least two normal donors were pooled, diluted in fresh medium to a final concentration of 1×10⁶ cells/mL, and plated in the interior wells of a 96 well round bottom microplate at 50 μL/well (5×10⁴ cells/well). Test dilutions were prepared at a 2× concentration in microtiter tubes and 100 μL of each concentration was placed in appropriate wells in a standard format. IL-28, IL-29 and MetIL-29C172S-PEG were added at concentrations from 0-10 μg/ml, usually in 1/2 log dilutions. 50 μL of a predetermined dilution of virus stock was placed in each test well (final MOI of 0.1). Wells with only cells and virus added were used for virus control. Separate plates were prepared identically without virus for drug cytotoxicity studies using an MTS assay system. The PBMC cultures were maintained for seven days following infection, at which time cell-free supernatant samples were collected and assayed for reverse transcriptase activity and p24 antigen levels.

A decrease in reverse transcriptase activity or p24 antigen levels with IL-28, IL-29 and MetIL-29C172S-PEG would be indicators of antiviral activity. Result would demonstrate that IL-28 and IL-29 may have therapeutic value in treating HIV and AIDS.

### Example 10

### IL-28 and IL-29 Inhibit GBV-B Replication in Marmoset Liver Cells

HCV is a member of the Flaviviridae family of RNA viruses. HCV does not replicate well in either ex-vivo or in vitro cultures and therefore, there are no satisfactory systems to test the anti-HCV activity of molecules in vitro. GB virus B (GBV-B) is an attractive surrogate model for use in the development of anti-HCV antiviral agents since it has a relatively high level of sequence identity with HCV and is a hepatotropic virus. To date, the virus can only be grown in the primary hepatocytes of certain non-human primates. This is accomplished by either isolating hepatocytes in vitro and infecting them with GBV-B, or by isolating hepatocytes from GBV-B infected marmosets and directly using them with antiviral compounds.

The effects of IL-28, IL-29 and MetIL-29C172S-PEG are assayed on GBV-B extracellular RNA production by TaqMan RT-PCR and on cytotoxicity using CellTiter96® reagent (Promega, Madison, Wis.) at six half-log dilutions IL-28, IL-29 or MetIL-29C172S-PEG polypeptide in triplicate. Untreated cultures serve as the cell and virus controls. Both RIBAVIRIN° (200 μg/ml at the highest test concentration) and IFN-α (5000 IU/ml at the highest test) are included as positive control compounds. Primary hepatocyte cultures are isolated and plated out on collagen-coated plates. The next day the cultures are treated with the test samples (IL-28, IL-29, MetIL-29C172S-PEG, IFNα, or RIBAVIRIN®) for 24 hr before being exposed to GBV-B virions or treated directly with test samples when using in vivo infected hepatocytes. Test samples and media are added the next day, and replaced three days later. Three to four days later (at day 6-7 post test sample addition) the supernatant is collected and the cell numbers quantitated with CellTiter96®. Viral RNA is extracted from the supernatant and quantified with triplicate replicates in a quantitative TaqMan RT-PCR assay using an in vitro transcribed RNA containing the RT-PCR target as a standard. The average of replicate samples is computed Inhibition of virus production is assessed by plotting the average RNA and cell number values of the triplicate samples relative to the untreated virus and cell controls. The inhibitory concentration of drug resulting in 50% inhibition of GBV-B RNA production (IC50) and the toxic concentration resulting in destruction of 50% of cell numbers relative to control values (TC50) are calculated by interpolation from graphs created with the data.

Inhibition of the GBV-B RNA production by IL-28 and 29 is an indication of the antiviral properties of IL-28 and IL-29 on this Hepatitis C-like virus on hepatocytes, the primary organ of infection of Hepatitis C, and positive results suggest that IL-28 or IL-29 may be useful in treating HCV infections in humans.

### Example 11

### IL-28, IL-29 and MetIL-29C172S-PEG Inhibit HBV Replication in WT10 Cells

Chronic hepatitis B (HBV) is one of the most common and severe viral infections of humans belonging to the Hepadnaviridae family of viruses. To test the antiviral activities of IL-28 and IL-29 against HBV, IL-28, IL-29 and MetIL-29C172S-PEG were tested against HBV in an in vitro infection system using a variant of the human liver line HepG2. IL-28, IL-29 and MetIL-29C172S-PEG inhibited viral replication in this system, suggesting therapeutic value in treating HBV in humans.

WT10 cells are a derivative of the human liver cell line HepG2 2.2.15. WT10 cells are stably transfected with the HBV genome, enabling stable expression of HBV transcripts in the cell line (Fu and Cheng, Antimicrobial Agents Chemother. 44(12):3402-3407, 2000). In the WT10 assay the drug in question and a 3TC control will be assayed at five concentrations each, diluted in a half-log series. The endpoints are TaqMan PCR for extracellular HBV DNA (IC50) and cell numbers using CellTiter96 reagent (TC50). The assay is similar to that described by Korba et al. Antiviral Res. 15(3):217-228, 1991 and Korba et al., Antiviral Res. 19(1):55-70, 1992. Briefly, WT10 cells are plated in 96-well microtiter plates. After 16-24 hours the confluent monolayer of HepG2-2.2.15 cells is washed and the medium is replaced with complete medium containing varying concentrations of a test samples in triplicate. 3TC is used as the positive control, while media alone is added to cells as a negative control (virus control, VC). Three days later the culture medium is replaced with fresh medium containing the appropriately diluted test samples. Six days following the initial addition of the test compound, the cell culture supernatant is collected, treated with pronase and DNAse, and used in a real-time quantitative TaqMan PCR assay. The PCR-amplified HBV DNA is detected in real-time by monitoring increases in fluorescence signals that result from the exonucleolytic degradation of a quenched fluorescent probe molecule that hybridizes to the amplified HBV DNA. For each PCR amplification, a standard curve is simultaneously generated using dilutions of purified HBV DNA. Antiviral activity is calculated from the reduction in HBV DNA levels (IC₅₀). A dye uptake assay is then employed to measure cell viability which is used to calculate toxicity (TC₅₀). The therapeutic index (TI) is calculated as TC₅₀/IC₅₀.

IL-28, IL-29 and MetIL-29C172S-PEG inhibited HepB viral replication in WT10 cells with an IC50 <0.032 ug/ml. This demonstrates antiviral activity of IL-28 and IL-29 against HBV grown in liver cell lines, providing evidence of therapeutic value for treating HBV in human patients.

### Example 12

### IL-28, IL-29 and MetIL-29C172S-PEG Inhibit BVDV Replication in Bovine Kidney Cells

HCV is a member of the Flaviviridae family of RNA viruses. Other viruses belonging to this family are the bovine viral diarrhea virus (BVDV) and yellow fever virus (YFV). HCV does not replicate well in either ex vivo or in vitro cultures and therefore there are no systems to test anti-HCV activity in vitro. The BVDV and YFV assays are used as surrogate viruses for HCV to test the antiviral activities against the Flavivirida family of viruses.

The antiviral effects of IL-28, IL-29 and MetIL-29C172S-PEG were assessed in inhibition of cytopathic effect assays (CPE). The assay measured cell death using Madin-Darby bovine kidney cells (MDBK) after infection with cytopathic BVDV virus and the inhibition of cell death by addition of IL-28, IL-29 and MetIL-29C172S-PEG. The MDBK cells were propagated in Dulbecco's modified essential medium (DMEM) containing phenol red with 10% horse serum, 1% glutamine and 1% penicillin-streptomycin. CPE inhibition assays were performed in DMEM without phenol red with 2% FBS, 1% glutamine and 1% Pen-Strep. On the day preceding the assays, cells were trypsinized (1% trypsin-EDTA), washed, counted and plated out at 10⁴ cells/well in a 96-well flat-bottom BioCoat® plates (Fisher Scientific, Pittsburgh, Pa.) in a volume of 100 μl/well. The next day, the medium was removed and a pre-titered aliquot of virus was added to the cells. The amount of virus was the maximum dilution that would yield complete cell killing (>80%) at the time of maximal CPE development (day 7 for BVDV). Cell viability was determined using a CellTiter96® reagent (Promega) according to the manufacturer's protocol, using a Vmax plate reader (Molecular Devices, Sunnyvale, Calif.). Test samples were tested at six concentrations each, diluted in assay medium in a half-log series. IFNα and RIBAVIRIN® were used as positive controls. Test sample were added at the time of viral infection. The average background and sample color-corrected data for percent CPE reduction and percent cell viability at each concentration were determined relative to controls and the IC₅₀ calculated relative to the TC₅₀.

IL-28, IL-29 and MetIL-29C172S-PEG inhibited cell death induced by BVDV in MDBK bovine kidney cells. IL-28 inhibited cell death with an IC₅₀ of 0.02 μg/ml, IL-29 inhibited cell death with an IC₅₀ of 0.19 μg/ml, and MetIL-29C172S-PEG inhibited cell death with an IC₅₀ of 0.45 μg/ml. This demonstrated that IL-28 and IL-29 have antiviral activity against the Flavivirida family of viruses.

### Example 13

### Induction of Interferon Stimulated Genes by IL-28 and IL-29

### A. Human Peripheral Blood Mononuclear Cells

Freshly isolated human peripheral blood mononuclear cells were grown in the presence of IL-29 (20 ng/mL), IFNα2a (2 ng/ml) (PBL Biomedical Labs, Piscataway, N.J.), or in medium alone. Cells were incubated for 6, 24, 48, or 72 hours, and then total RNA was isolated and treated with RNase-free DNase. 100 ng total RNA was used as a template for One-Step Semi-Quantitative RT-PCR® using Taqman One-Step RT-PCR Master Mix® Reagents and gene specific primers as suggested by the manufacturer. (Applied Biosystems, Branchburg, N.J.) Results were normalized to HPRT and are shown as the fold induction over the medium alone control for each time-point. Table 15 shows that IL-29 induces Interferon Stimulated Gene Expression in human peripheral blood mononuclear cells at all time-points tested.

TABLE 15

| | MxA Fold | Pkr Fold | OAS Fold |
|---|---|---|---|
| | induction | Induction | Induction |
| 6 hr IL29 | 3.1 | 2.1 | 2.5 |
| 6 hr IFNα2a | 17.2 | 9.6 | 16.2 |
| 24 hr IL29 | 19.2 | 5.0 | 8.8 |
| 24 hr IFNα2a | 57.2 | 9.4 | 22.3 |
| 48 hr IL29 | 7.9 | 3.5 | 3.3 |
| 48 hr IFNα2a | 18.1 | 5.0 | 17.3 |
| 72 hr IL29 | 9.4 | 3.7 | 9.6 |
| 72 hr IFNα2a | 29.9 | 6.4 | 47.3 |

### B. Activated Human T Cells

Human T cells were isolated by negative selection from freshly harvested peripheral blood mononuclear cells using the Pan T-cell Isolation® kit according to manufacturer's instructions (Miltenyi, Auburn, Calif.). T cells were then activated and expanded for 5 days with plate-bound anti-CD3, soluble anti-CD28 (0.5 ug/ml), (Pharmingen, San Diego, Calif.) and Interleukin 2 (IL-2; 100 U/ml) (R&D Systems, Minneapolis, Minn.), washed and then expanded for a further 5 days with IL-2. Following activation and expansion, cells were stimulated with IL-28A (20 ng/ml), IL-29 (20 ng/ml), or medium alone for 3, 6, or 18 hours. Total RNA was isolated and treated with RNase-Free DNase. One-Step Semi-Quantitative RT-PCR® was performed as described in the example above. Results were normalized to HPRT and are shown as the fold induction over the medium alone control for each time-point. Table 16 shows that IL-28 and IL-29 induce Interferon Stimulated Gene expression in activated human T cells at all time-points tested.

TABLE 16

| | MxA Fold | Pkr Fold | OAS Fold |
|---|---|---|---|
| | Induction | Induction | Induction |
| Donor #1 3 hr IL28 | 5.2 | 2.8 | 4.8 |
| Donor #1 3 hr IL29 | 5.0 | 3.5 | 6.0 |
| Donor #1 6 hr IL28 | 5.5 | 2.2 | 3.0 |
| Donor #1 6 hr IL29 | 6.4 | 2.2 | 3.7 |
| Donor #1 18 hr IL28 | 4.6 | 4.8 | 4.0 |
| Donor #1 18 hr IL29 | 5.0 | 3.8 | 4.1 |
| Donor #2 3 hr IL28 | 5.7 | 2.2 | 3.5 |
| Donor #2 3 hr IL29 | 6.2 | 2.8 | 4.7 |
| Donor #2 6 hr IL28 | 7.3 | 1.9 | 4.4 |
| Donor #2 6 hr IL29 | 8.7 | 2.6 | 4.9 |
| Donor #2 18 hr IL28 | 4.7 | 2.3 | 3.6 |
| Donor #2 18 hr IL29 | 4.9 | 2.1 | 3.8 |

### C. Primary Human Hepatocytes

Freshly isolated human hepatocytes from two separate donors (Cambrex, Baltimore, Md. and CellzDirect, Tucson, Ariz.) were stimulated with IL-28A (50 ng/ml), IL-29 (50 ng/ml), IFNα2a (50 ng/ml), or medium alone for 24 hours. Following stimulation, total RNA was isolated and treated with RNase-Free DNase. One-step semi-quantitative RT-PCR was performed as described previously in the example above. Results were normalized to HPRT and are shown as the fold induction over the medium alone control for each time-point. Table 17 shows that IL-28 and IL-29 induce Interferon Stimulated Gene expression in primary human hepatocytes following 24-hour stimulation.

TABLE 17

| | MxA Fold | Pkr Fold | OAS Fold |
|---|---|---|---|
| | Induction | Induction | Induction |
| Donor #1 IL28 | 31.4 | 6.4 | 30.4 |
| Donor #1 IL29 | 31.8 | 5.2 | 27.8 |
| Donor #1 IFN- | 63.4 | 8.2 | 66.7 |
| α2a | | | |
| Donor #2 IL28 | 41.7 | 4.2 | 24.3 |
| Donor #2 IL29 | 44.8 | 5.2 | 25.2 |
| Donor #2 IFN- | 53.2 | 4.8 | 38.3 |
| α2a | | | |

### D. HepG2 and HuH7: Human Liver Hepatoma Cell Lines

HepG2 and HuH7 cells (ATCC NOS. 8065, Manassas, Va.) were stimulated with IL-28A (10 ng/ml), IL-29 (10 ng/ml), IFNα2a (10 ng/ml), IFNB (1 ng/ml) (PBL Biomedical, Piscataway, N.J.), or medium alone for 24 or 48 hours. In a separate culture, HepG2 cells were stimulated as described above with 20 ng/ml of MetIL-29C172S-PEG or MetIL-29-PEG. Total RNA was isolated and treated with RNase-Free DNase. 100 ng Total RNA was used as a template for one-step semi-quantitative RT-PCR as described previously. Results were normalized to HPRT and are shown as the fold induction over the medium alone control for each time-point. Table 18 shows that IL-28 and IL-29 induce ISG expression in HepG2 and HuH7 liver hepatoma cell lines after 24 and 48 hours.

TABLE 18

| | MxA Fold | Pkr Fold | |
|---|---|---|---|
| | Induction | Induction | OAS Fold Induction |
| HepG2 24 hr IL28 | 12.4 | 0.7 | 3.3 |
| HepG2 24 hr IL29 | 36.6 | 2.2 | 6.4 |
| HepG2 24 hr IFNα2a | 12.2 | 1.9 | 3.2 |
| HepG2 24 hr IFNβ | 93.6 | 3.9 | 19.0 |
| HepG2 48 hr IL28 | 2.7 | 0.9 | 1.1 |
| HepG2 48 hr IL29 | 27.2 | 2.1 | 5.3 |
| HepG2 48 hr IFNα2a | 2.5 | 0.9 | 1.2 |
| HepG2 48 hr IFNβ | 15.9 | 1.8 | 3.3 |
| HuH7 24 hr IL28 | 132.5 | 5.4 | 52.6 |
| HuH7 24 hr IL29 | 220.2 | 7.0 | 116.6 |
| HuH7 24 hr IFNα2a | 157.0 | 5.7 | 67.0 |
| HuH7 24 hr IFNβ | 279.8 | 5.6 | 151.8 |
| HuH7 48 hr IL28 | 25.6 | 3.4 | 10.3 |
| HuH7 48 hr IL29 | 143.5 | 7.4 | 60.3 |
| HuH7 48 hr IFNα2a | 91.3 | 5.8 | 32.3 |
| HuH7 48 hr IFNβ | 65.0 | 4.2 | 35.7 |

TABLE 19

| | MxA Fold | OAS Fold | Pkr Fold |
|---|---|---|---|
| | Induction | Induction | Induction |
| MetIL-29-PEG | 36.7 | 6.9 | 2.2 |
| MetIL-29C172S-PEG | 46.1 | 8.9 | 2.8 |

Data shown is for 20 ng/ml metIL-29-PEG and metIL-29C172S-PEG versions of IL-29 after culture for 24 hours.

Data shown is normalized to HPRT and shown as fold induction over unstimulated cells.

### Example 14

### Antiviral Activity of IL-28 and IL-29 in HCV Replicon System

The ability of antiviral drugs to inhibit HCV replication can be tested in vitro with the HCV replicon system. The replicon system consists of the Huh7 human hepatoma cell line that has been transfected with subgenomic RNA replicons that direct constitutive replication of HCV genomic RNAs (Blight, K. J. et al. Science 290:1972-1974, 2000). Treatment of replicon clones with IFNα at 10 IU/ml reduces the amount of HCV RNA by 85% compared to untreated control cell lines. The ability of IL-28A and IL-29 to reduce the amount of HCV RNA produced by the replicon clones in 72 hours indicates the antiviral state conferred upon Huh7 cells by IL-28A/IL-29 treatment is effective in inhibiting HCV replicon replication, and thereby, very likely effective in inhibiting HCV replication.

The ability of IL-28A and IL-29 to inhibit HCV replication as determined by Bayer Branched chain DNA kit, is be done under the following conditions:

IL28 alone at increasing concentrations (6)* up to 1.0 μg/ml

IL29 alone at increasing concentrations (6)* up to 1.0 μg/ml

PEGIL29 alone at increasing concentrations (6)* up to 1.0 μg/ml

IFNα2A alone at 0.3, 1.0, and 3.0 IU/ml

Ribavirin alone.

The positive control is IFNα and the negative control is ribavirin.

The cells are stained after 72 hours with Alomar Blue to assess viability.

*The concentrations for conditions 1-3 are:

μg/ml, 0.32 μg/ml, 0.10 μg/ml, 0.032 μg/ml, 0.010 μg/ml, 0.0032 μg/ml.

The replicon clone (BB7) is treated 1× per day for 3 consecutive days with the doses listed above. Total HCV RNA is measured after 72 hours.

### Example 15

### IL-28 and IL-29 have Antiviral Activity Against Pathogenic Viruses

Two methods are used to assay in vitro antiviral activity of IL-28 and IL-29 against a panel of pathogenic viruses including, among others, adenovirus, parainfluenza virus. respiratory syncytial virus, rhino virus, coxsackie virus, influenza virus, vaccinia virus, west nile virus, dengue virus, venezuelan equine encephalitis virus, pichinde virus and polio virus. These two methods are inhibition of virus-induced cytopathic effect (CPE) determined by visual (microscopic) examination of the cells and increase in neutral red (NR) dye uptake into cells. In the CPE inhibition method, seven concentrations of test drug (log 10 dilutions, such as 1000, 100, 10, 1, 0.1, 0.01, 0.001 ng/ml) are evaluated against each virus in 96-well flat-bottomed microplates containing host cells. The compounds are added 24 hours prior to virus, which is used at a concentration of approximately 5 to 100 cell culture infectious doses per well, depending upon the virus, which equates to a multiplicity of infection (MOI) of 0.01 to 0.0001 infectious particles per cell. The tests are read after incubation at 37° C. for a specified time sufficient to allow adequate viral cytopathic effect to develop. In the NR uptake assay, dye (0.34% concentration in medium) is added to the same set of plates used to obtain the visual scores. After 2 h, the color intensity of the dye absorbed by and subsequently eluted from the cells is determined using a microplate autoreader. Antiviral activity is expressed as the 50% effective (virus-inhibitory) concentration (EC50) determined by plotting compound concentration versus percent inhibition on semilogarithmic graph paper. The EC50/IC50 data in some cases may be determined by appropriate regression analysis software. In general, the EC50s determined by NR assay are two- to fourfold higher than those obtained by the CPE method.

TABLE 20
Visual Assay

| | | | | | | SI |
|---|---|---|---|---|---|---|
| | | | | | | Visual |
| | | | | | | (IC50/ |
| Virus | Cell line | Drug | EC50 Visual | | IC50 Visual | EC50) |
| Adenovirus | A549 | IL-28A | >10 | μg/ml | >10 μg/ml | 0 |
| Adenovirus | A549 | IL-29 | >10 | μg/ml | >10 μg/ml | 0 |
| Adenovirus | A549 | MetIL-29C172S- | >10 | μg/ml | >10 μg/ml | 0 |
| | | PEG | | | | |
| Parainfluenza | MA-104 | IL-28A | >10 | μg/ml | >10 μg/ml | 0 |
| virus | | | | | | |
| Parainfluenza | MA-104 | IL-29 | >10 | μg/ml | >10 μg/ml | 0 |
| virus | | | | | | |
| Parainfluenza | MA-104 | MetIL-29C172S- | >10 | μg/ml | >10 μg/ml | 0 |
| virus | | PEG | | | | |
| Respiratory | MA-104 | IL-28A | >10 | μg/ml | >10 μg/ml | 0 |
| syncytial | | | | | | |
| virus | | | | | | |
| Respiratory | MA-104 | IL-29 | >10 | μg/ml | >10 μg/ml | 0 |
| syncytial | | | | | | |
| virus | | | | | | |
| Respiratory | MA-104 | MetIL-29C172S- | >10 | μg/ml | >10 μg/ml | 0 |
| syncytial | | PEG | | | | |
| virus | | | | | | |
| Rhino 2 | KB | IL-28A | >10 | μg/ml | >10 μg/ml | 0 |
| Rhino 2 | KB | IL-29 | >10 | μg/ml | >10 μg/ml | 0 |
| Rhino 2 | KB | MetIL-29C172S- | >10 | μg/ml | >10 μg/ml | 0 |
| | | PEG | | | | |
| Rhino 9 | HeLa | IL-28A | >10 | μg/ml | >10 μg/ml | 0 |
| Rhino 9 | HeLa | IL-29 | >10 | μg/ml | >10 μg/ml | 0 |
| Rhino 9 | HeLa | MetIL-29C172S- | >10 | μg/ml | >10 μg/ml | 0 |
| | | PEG | | | | |
| Coxsackie | KB | IL-28A | >10 | μg/ml | >10 μg/ml | 0 |
| B4 virus | | | | | | |
| Coxsackie | KB | IL-29 | >10 | μg/ml | >10 μg/ml | 0 |
| B4 virus | | | | | | |
| Coxsackie | KB | MetIL-29C172S- | >10 | μg/ml | >10 μg/ml | 0 |
| B4 virus | | PEG | | | | |
| Influenza | Maden- | IL-28A | >10 | μg/ml | >10 μg/ml | 0 |
| (type A | Darby | | | | | |
| [H3N2]) | Canine | | | | | |
| | Kidney | | | | | |
| Influenza | Maden- | IL-29 | >10 | μg/ml | >10 μg/ml | 0 |
| (type A | Darby | | | | | |
| [H3N2]) | Canine | | | | | |
| | Kidney | | | | | |
| Influenza | Maden- | MetIL-29C172S- | >10 | μg/ml | >10 μg/ml | 0 |
| (type A | Darby | PEG | | | | |
| [H3N2]) | Canine | | | | | |
| | Kidney | | | | | |
| Influenza | Vero | IL-28A | 0.1 | μg/ml | >10 μg/ml | >100 |
| (type A | | | | | | |
| [H3N2]) | | | | | | |
| Influenza | Vero | IL-29 | >10 | μg/ml | >10 μg/ml | 0 |
| (type A | | | | | | |
| [H3N2]) | | | | | | |
| Influenza | Vero | MetIL-29C172S- | 0.045 | μg/ml | >10 μg/ml | >222 |
| (type A | | PEG | | | | |
| [H3N2]) | | | | | | |
| Vaccinia | Vero | IL-28A | >10 | μg/ml | >10 μg/ml | 0 |
| virus | | | | | | |
| Vaccinia | Vero | IL-29 | >10 | μg/ml | >10 μg/ml | 0 |
| virus | | | | | | |
| Vaccinia | Vero | MetIL-29C172S- | >10 | μg/ml | >10 μg/ml | 0 |
| virus | | PEG | | | | |
| West Nile | Vero | IL-28A | 0.00001 | μg/ml | >10 μg/ml | >1,000,000 |
| virus | | | | | | |
| West Nile | Vero | IL-29 | 0.000032 | μg/ml | >10 μg/ml | >300,000 |
| virus | | | | | | |
| West Nile | Vero | MetIL-29C172S- | 0.001 | μg/ml | >10 μg/ml | >10,000 |
| virus | | PEG | | | | |
| Dengue virus | Vero | IL-28A | 0.01 | μg/ml | >10 μg/ml | >1000 |
| Dengue virus | Vero | IL-29 | 0.032 | μg/ml | >10 μg/ml | >312 |
| Dengue virus | Vero | MetIL-29C172S- | 0.0075 | μg/ml | >10 μg/ml | >1330 |
| | | PEG | | | | |
| Venezuelan | Vero | IL-28A | 0.01 | μg/ml | >10 μg/ml | >1000 |
| equine | | | | | | |
| encephalitis | | | | | | |
| virus | | | | | | |
| Venezuelan | Vero | IL-29 | 0.012 | μg/ml | >10 μg/ml | >833 |
| equine | | | | | | |
| encephalitis | | | | | | |
| virus | | | | | | |
| Venezuelan | Vero | MetIL-29C172S- | 0.0065 | μg/ml | >10 μg/ml | >1538 |
| equine | | PEG | | | | |
| encephalitis | | | | | | |
| virus | | | | | | |
| Pichinde | BSC-1 | IL-28A | >10 | μg/ml | >10 μg/ml | 0 |
| virus | | | | | | |
| Pichinde | BSC-1 | IL-29 | >10 | μg/ml | >10 μg/ml | 0 |
| virus | | | | | | |
| Pichinde | BSC-1 | MetIL-29C172S- | >10 | μg/ml | >10 μg/ml | 0 |
| virus | | PEG | | | | |
| Polio virus | Vero | IL-28A | >10 | μg/ml | >10 μg/ml | 0 |
| Polio virus | Vero | IL-29 | >10 | μg/ml | >10 μg/ml | 0 |
| Polio virus | Vero | MetIL-29C172S- | >10 | μg/ml | >10 μg/ml | 0 |
| | | PEG | | | | |

TABLE 21
Neutral Red Assay

| | | | | | | SI NR |
|---|---|---|---|---|---|---|
| | | | | | | (IC50/ |
| Virus | Cell line | Drug | EC50 NR | | IC50 NR | EC50) |
| Adenovirus | A549 | IL-28A | >10 | μg/ml | >10 μg/ml | 0 |
| Adenovirus | A549 | IL-29 | >10 | μg/ml | >10 μg/ml | 0 |
| Adenovirus | A549 | MetIL-29C172S- | >10 | μg/ml | >10 μg/ml | 0 |
| | | PEG | | | | |
| Parainfluenza | MA-104 | IL-28A | >10 | μg/ml | >10 μg/ml | 0 |
| virus | | | | | | |
| Parainfluenza | MA-104 | IL-29 | >10 | μg/ml | >10 μg/ml | 0 |
| virus | | | | | | |
| Parainfluenza | MA-104 | MetIL-29C172S- | >10 | μg/ml | >10 μg/ml | 0 |
| virus | | PEG | | | | |
| Respiratory | MA-104 | IL-28A | >10 | μg/ml | >10 μg/ml | 0 |
| syncytial virus | | | | | | |
| Respiratory | MA-104 | IL-29 | >10 | μg/ml | >10 μg/ml | 0 |
| syncytial virus | | | | | | |
| Respiratory | MA-104 | MetIL-29C172S- | 5.47 | μg/ml | >10 μg/ml | >2 |
| syncytial virus | | PEG | | | | |
| Rhino 2 | KB | IL-28A | >10 | μg/ml | >10 μg/ml | 0 |
| Rhino 2 | KB | IL-29 | >10 | μg/ml | >10 μg/ml | 0 |
| Rhino 2 | KB | MetIL-29C172S- | >10 | μg/ml | >10 μg/ml | 0 |
| | | PEG | | | | |
| Rhino 9 | HeLa | IL-28A | 1.726 | μg/ml | >10 μg/ml | >6 |
| Rhino 9 | HeLa | IL-29 | 0.982 | μg/ml | >10 μg/ml | >10 |
| Rhino 9 | HeLa | MetIL-29C172S- | 2.051 | μg/ml | >10 μg/ml | >5 |
| | | PEG | | | | |
| Coxsackie B4 | KB | IL-28A | >10 | μg/ml | >10 μg/ml | 0 |
| virus | | | | | | |
| Coxsackie B4 | KB | IL-29 | >10 | μg/ml | >10 μg/ml | 0 |
| virus | | | | | | |
| Coxsackie B4 | KB | MetIL-29C172S- | >10 | μg/ml | >10 μg/ml | 0 |
| virus | | PEG | | | | |
| Influenza (type | Maden- | IL-28A | >10 | μg/ml | >10 μg/ml | 0 |
| A [H3N2]) | Darby | | | | | |
| | Canine | | | | | |
| | Kidney | | | | | |
| Influenza (type | Maden- | IL-29 | >10 | μg/ml | >10 μg/ml | 0 |
| A [H3N2]) | Darby | | | | | |
| | Canine | | | | | |
| | Kidney | | | | | |
| Influenza (type | Maden- | MetIL-29C172S- | >10 | μg/ml | >10 μg/ml | 0 |
| A [H3N2]) | Darby | PEG | | | | |
| | Canine | | | | | |
| | Kidney | | | | | |
| Influenza (type | Vero | IL-28A | 0.25 | μg/ml | >10 μg/ml | >40 |
| A [H3N2]) | | | | | | |
| Influenza (type | Vero | IL-29 | 2 | μg/ml | >10 μg/ml | >5 |
| A [H3N2]) | | | | | | |
| Influenza (type | Vero | MetIL-29C172S- | 1.4 | μg/ml | >10 μg/ml | >7 |
| A [H3N2]) | | PEG | | | | |
| Vaccinia virus | Vero | IL-28A | >10 | μg/ml | >10 μg/ml | 0 |
| Vaccinia virus | Vero | IL-29 | >10 | μg/ml | >10 μg/ml | 0 |
| Vaccinia virus | Vero | MetIL-29C172S- | >10 | μg/ml | >10 μg/ml | 0 |
| | | PEG | | | | |
| West Nile virus | Vero | IL-28A | 0.0001 | μg/ml | >10 μg/ml | >100,000 |
| West Nile virus | Vero | IL-29 | 0.00025 | μg/ml | >10 μg/ml | >40,000 |
| West Nile virus | Vero | MetIL-29C172S- | 0.00037 | μg/ml | >10 μg/ml | >27,000 |
| | | PEG | | | | |
| Dengue virus | Vero | IL-28A | 0.1 | μg/ml | >10 μg/ml | >100 |
| Dengue virus | Vero | IL-29 | 0.05 | μg/ml | >10 μg/ml | >200 |
| Dengue virus | Vero | MetIL-29C172S- | 0.06 | μg/ml | >10 μg/ml | >166 |
| | | PEG | | | | |
| Venezuelan | Vero | IL-28A | 0.035 | μg/ml | >10 μg/ml | >286 |
| equine | | | | | | |
| encephalitis | | | | | | |
| virus | | | | | | |
| Venezuelan | Vero | IL-29 | 0.05 | μg/ml | >10 μg/ml | >200 |
| equine | | | | | | |
| encephalitis | | | | | | |
| virus | | | | | | |
| Venezuelan | Vero | MetIL-29C172S- | 0.02 | μg/ml | >10 μg/ml | >500 |
| equine | | PEG | | | | |
| encephalitis | | | | | | |
| virus | | | | | | |
| Pichinde virus | BSC-1 | IL-28A | >10 | μg/ml | >10 μg/ml | 0 |
| Pichinde virus | BSC-1 | IL-29 | >10 | μg/ml | >10 μg/ml | 0 |
| Pichinde virus | BSC-1 | MetIL-29C172S- | >10 | μg/ml | >10 μg/ml | 0 |
| | | PEG | | | | |
| Polio virus | Vero | IL-28A | >1.672 | μg/ml | >10 μg/ml | >6 |
| Polio virus | Vero | IL-29 | >10 | μg/ml | >10 μg/ml | 0 |
| Polio virus | Vero | MetIL-29C172S- | >10 | μg/ml | >10 μg/ml | 0 |
| | | PEG | | | | |

### Example 16

### IL-28, IL-29, metIL-29-PEG and metIL-29C172S-PEG Stimulate ISG Induction in the Mouse Liver Cell Line AML-12

Interferon stimulated genes (ISGs) are genes that are induced by type I interferons (IFNs) and also by the IL-28 and IL-29 family molecules, suggesting that IFN and IL-28 and IL-29 induce similar pathways leading to antiviral activity. Human type I IFNs (IFNα1-4 and IFNβ) have little or no activity on mouse cells, which is thought to be caused by lack of species cross-reactivity. To test if human IL-28 and IL-29 have effects on mouse cells, ISG induction by human IL-28 and IL-29 was evaluated by real-time PCR on the mouse liver derived cell line AML-12.

AML-12 cells were plated in 6-well plates in complete DMEM media at a concentration of 2×10⁶ cells/well. Twenty-four hours after plating cells, human IL-28 and IL-29 were added to the culture at a concentration of 20 ng/ml. As a control, cells were either stimulated with mouse IFNα (positive control) or unstimulated (negative). Cells were harvested at 8, 24, 48 and 72 hours after addition of CHO-derived human IL-28A (SEQ ID NO:18) or IL-29 (SEQ ID NO:20). RNA was isolated from cell pellets using RNAEasy-kit® (Qiagen, Valencia, Calif.). RNA was treated with DNase (Millipore, Billerica, Mass.) to clean RNA of any contaminating DNA. cDNA was generated using Perkin-Elmer RT mix. ISG gene induction was evaluated by real-time PCR using primers and probes specific for mouse OAS, Pkr and Mx1. To obtain quantitative data, HPRT real-time PCR was duplexed with ISG PCR. A standard curve was obtained using known amounts of RNA from IFN-stimulated mouse PBLs. All data are shown as expression relative to internal HPRT expression.

Human IL-28A and IL-29 stimulated ISG induction in the mouse hepatocyte cell line AML-12 and demonstrated that unlike type I IFNs, the IL-28/29 family proteins showed cross-species reactivity.

TABLE 22

| Stimulation | OAS | PkR | Mx1 |
|---|---|---|---|
| None | 0.001 | 0.001 | 0.001 |
| Human IL-28 | 0.04 | 0.02 | 0.06 |
| Human IL-29 | 0.04 | 0.02 | 0.07 |
| Mouse IL-28 | 0.04 | 0.02 | 0.08 |
| Mouse IFNα | 0.02 | 0.02 | 0.01 |

All data shown were expressed as fold relative to HPRT gene expression ng of OAS mRNA=normalized value of OAS mRNA amount relative to internal ng of HPRT mRNA housekeeping gene, HPRT

As an example, the data for the 48 hour time point is shown.

TABLE 23
AML12's

| | Mx1 Fold | OAS Fold | Pkr Fold |
|---|---|---|---|
| | Induction | Induction | Induction |
| MetIL-29-PEG | 728 | 614 | 8 |
| MetIL-29C172S-PEG | 761 | 657 | 8 |

Cells were stimulated with 20 ng/ml metIL-29-PEG or metIL-29C172S-PEG for 24 hours.

Data shown is normalized to HPRT and shown as fold induction over unstimulated cells.

### Example 17

### ISGs are Efficiently Induced in Spleens of Transgenic Mice Expressing Human IL-29

Transgenic (Tg) mice were generated expressing human IL-29 under the control of the Eu-lck promoter. To study if human IL-29 has biological activity in vivo in mice, expression of ISGs was analyzed by real-time PCR in the spleens of Eu-lck IL-29 transgenic mice.

Transgenic mice (C3H/C57BL/6) were generated using a construct that expressed the human IL-29 gene under the control of the Eu-lck promoter. This promoter is active in T cells and B cells. Transgenic mice and their non-transgenic littermates (n=2/gp) were sacrificed at about 10 weeks of age. Spleens of mice were isolated. RNA was isolated from cell pellets using RNAEasy-kit®(Qiagen). RNA was treated with DNase to clean RNA of any contaminating DNA. cDNA was generated using Perkin-Elmer RT® mix. ISG gene induction was evaluated by real-time PCR using primers and probes (5′ FAM, 3′ NFQ) specific for mouse OAS, Pkr and Mx1. To obtain quantitative data, HPRT real-time PCR was duplexed with ISG PCR. Furthermore, a standard curve was obtained using known amounts of IFN stimulated mouse PBLs. All data are shown as expression relative to internal HPRT expression.

Spleens isolated from IL-29 Tg mice showed high induction of ISGs OAS, Pkr and Mx1 compared to their non-Tg littermate controls suggesting that human IL-29 is biologically active in vivo in mice.

TABLE 24

| Mice | OAS | PkR | Mx1 |
|---|---|---|---|
| Non-Tg | 4.5 | 4.5 | 3.5 |
| IL-29 Tg | 12 | 8 | 21 |

All data shown are fold expression relative to HPRT gene expression. The average expression in two mice is shown

### Example 18

### Human IL-28 and IL-29 Protein Induce ISG Gene Expression in Liver, Spleen and Blood of Mice

To determine whether human IL-28 and IL-29 induce interferon stimulated genes in vivo, CHO-derived human IL-28A and IL-29 protein were injected into mice. In addition, E. coli derived IL-29 was also tested in in vivo assays as described above using MetIL-29C172S-PEG and MetIL-29-PEG. At various time points and at different doses, ISG gene induction was measured in the blood, spleen and livers of the mice.

C57BL/6 mice were injected i.p or i.v with a range of doses (10 μg-250 μg) of CHO-derived human IL-28A and IL-29 or MetIL-29C172S-PEG and MetIL-29C16-C113-PEG. Mice were sacrificed at various time points (1 hr-48 hr). Spleens and livers were isolated from mice, and RNA was isolated. RNA was also isolated from the blood cells. The cells were pelleted and RNA isolated from pellets using RNAEasy®-kit (Qiagen). RNA was treated with DNase (Amicon) to rid RNA of any contaminating DNA. cDNA was generated using Perkin-Elmer RT mix (Perkin-Elmer). ISG gene induction was measured by real-time PCR using primers and probes specific for mouse OAS, Pkr and Mx1. To obtain quantitative data, HPRT real-time PCR was duplexed with ISG PCR. A standard curve was calculated using known amounts of IFN-stimulated mouse PBLs. All data are shown as expression relative to internal HPRT expression.

Human IL-29 induced ISG gene expression (OAS, Pkr, Mx1) in the livers, spleen and blood of mice in a dose dependent manner. Expression of ISGs peaked between 1-6 hours after injection and showed sustained expression above control mice up to 48 hours. In this experiment, human IL-28A did not induce ISG gene expression.

TABLE 25

| Injection | OAS-1 hr | OAS-6 hr | OAS-24 hr | OAS-48 hr |
|---|---|---|---|---|
| None - liver | 1.6 | 1.6 | 1.6 | 1.6 |
| IL-29 liver | 2.5 | 4 | 2.5 | 2.8 |
| None - spleen | 1.8 | 1.8 | 1.8 | 1.8 |
| IL-29 - spleen | 4 | 6 | 3.2 | 3.2 |
| None - blood | 5 | 5 | 5 | 5 |
| IL-29 blood | 12 | 18 | 11 | 10 |

Results shown are fold expression relative to HPRT gene expression. A sample data set for IL-29 induced OAS in liver at a single injection of 250 μg i.v. is shown. The data shown is the average expression from 5 different animals/group.

TABLE 26

| Injection | OAS (24 hr) |
|---|---|
| None | 1.8 |
| IL-29 10 μg | 3.7 |
| IL-29 50 μg | 4.2 |
| IL-29 250 μg | 6 |

TABLE 27

| | MetIL-29-PEG | | | | MetIL-29C172S-PEG | | | | Naive |
|---|---|---|---|---|---|---|---|---|---|
| | 3 hr | 6 hr | 12 hr | 24 hr | 3 hr | 6 hr | 12 hr | 24 hr | 24 hr |
| PKR | 18.24 | 13.93 | 4.99 | 3.77 | 5.29 | 5.65 | 3.79 | 3.55 | 3.70 |
| OAS | 91.29 | 65.93 | 54.04 | 20.81 | 13.42 | 13.02 | 10.54 | 8.72 | 6.60 |
| Mx1 | 537.51 | 124.99 | 33.58 | 35.82 | 27.89 | 29.34 | 16.61 | 0.00 | 10.98 |

Mice were injected with 100 μg of proteins i.v. Data shown is fold expression over HPRT expression from livers of mice. Similar data was obtained from blood and spleens of mice.

### Example 19

### IL-28 and IL-29 Induce ISG Protein in Mice

To analyze of the effect of human IL-28 and IL-29 on induction of ISG protein (OAS), serum and plasma from IL-28 and IL-29 treated mice were tested for OAS activity.

C57BL/6 mice were injected i.v with PBS or a range of concentrations (10 μg-250 μg) of human IL-28 or IL-29. Serum and plasma were isolated from mice at varying time points, and OAS activity was measured using the OAS radioimmunoassay (RIA) kit from Eiken Chemicals (Tokyo, Japan).

IL-28 and IL-29 induced OAS activity in the serum and plasma of mice showing that these proteins are biologically active in vivo.

TABLE 28

| Injection | OAS-1 hr | OAS-6 hr | OAS-24 hr | OAS-48 hr |
|---|---|---|---|---|
| None | 80 | 80 | 80 | 80 |
| IL-29 | 80 | 80 | 180 | 200 |

OAS activity is shown at pmol/dL of plasma for a single concentration (250 μg) of human IL-29.

### Example 20

### IL-28 and IL-29 Inhibit Adenoviral Pathology in Mice

To test the antiviral activities of IL-28 and IL-29 against viruses that infect the liver, the test samples were tested in mice against infectious adenoviral vectors expressing an internal green fluorescent protein (GFP) gene. When injected intravenously, these viruses primarily target the liver for gene expression. The adenoviruses are replication deficient, but cause liver damage due to inflammatory cell infiltrate that can be monitored by measurement of serum levels of liver enzymes like AST and ALT, or by direct examination of liver pathology.

C57Bl/6 mice were given once daily intraperitoneal injections of 50 μg mouse IL-28 (zcyto24) or metIL-29C172S-PEG for 3 days. Control animals were injected with PBS. One hour following the 3^{rd} dose, mice were given a single bolus intravenous tail vein injection of the adenoviral vector, AdGFP (1×10⁹ plaque-forming units (pfu)). Following this, every other day mice were given an additional dose of PBS, mouse IL-28 or metIL-29C172S-PEG for 4 more doses (total of 7 doses). One hour following the final dose of PBS, mouse IL-28 or metIL-29C172S-PEG mice were terminally bleed and sacrificed. The serum and liver tissue were analyzed. Serum was analyzed for AST and ALT liver enzymes. Liver was isolated and analyzed for GFP expression and histology. For histology, liver specimens were fixed in formalin and then embedded in paraffin followed by H&E staining Sections of liver that had been blinded to treat were examined with a light microscope. Changes were noted and scored on a scale designed to measure liver pathology and inflammation.

Mouse IL-28 and IL-29 inhibited adenoviral infection and gene expression as measured by liver fluorescence. PBS-treated mice (n=8) had an average relative liver fluorescence of 52.4 (arbitrary units). In contrast, IL-28-treated mice (n=8) had a relative liver fluorescence of 34.5, and IL-29-treated mice (n=8) had a relative liver fluorescence of 38.9. A reduction in adenoviral infection and gene expression led to a reduced liver pathology as measured by serum ALT and AST levels and histology. PBS-treated mice (n=8) had an average serum AST of 234 U/L (units/liter) and serum ALT of 250 U/L. In contrast, IL-28-treated mice (n=8) had an average serum AST of 193 U/L and serum ALT of 216 U/L, and IL-29-treated mice (n=8) had an average serum AST of 162 U/L and serum ALT of 184 U/L. In addition, the liver histology indicated that mice given either mouse IL-28 or IL-29 had lower liver and inflammation scores than the PBS-treated group. The livers from the IL-29 group also had less proliferation of sinusoidal cells, fewer mitotic figures and fewer changes in the hepatocytes (e.g. vacuolation, presence of multiple nuclei, hepatocyte enlargement) than in the PBS treatment group. These data demonstrate that mouse IL-28 and IL-29 have antiviral properties against a liver-trophic virus.

### Example 21

### LCMV Models

Lymphocytic choriomeningitis virus (LCMV) infections in mice are an excellent model of acture and chronic infection. These models are used to evaluate the effect of cytokines on the antiviral immune response and the effects IL-28 and IL-29 have viral load and the antiviral immune response. The two models used are: LCMV Armstrong (acute) infection and LCMV Clone 13 (chronic) infection. (See, e.g., Wherry et al., J. Virol. 77:4911-4927, 2003; Blattman et al., Nature Med. 9(5):540-547, 2003; Hoffman et al., J. Immunol. 170:1339-1353, 2003.) There are three stages of CD8 T cell development in response to virus: 1) expansion, 2) contraction, and 3) memory (acute model). IL-28 or IL-29 is injected during each stage for both acute and chronic models. In the chronic model, IL-28 or IL-29 is injected 60 days after infection to assess the effect of IL-28 or IL-29 on persistent viral load. For both acute and chronic models, IL-28 or IL-29 is injected, and the viral load in blood, spleen and liver is examined. Other parameter that can be examined include: tetramer staining by flow to count the number of LCMV-specific CD8+ T cells; the ability of tetramer+ cells to produce cytokines when stimulated with their cognate LCMV antigen; and the ability of LCMV-specific CD8+ T cells to proliferate in response to their cognate LCMV antigen. LCMV-specific T cells are phenotyped by flow cytometry to assess the cells activation and differentiation state. Also, the ability of LCMV-specific CTL to lyse target cells bearing their cognate LCMV antigen is examined. The number and function of LCMV-specific CD4+ T cells is also assessed.

A reduction in viral load after treatment with IL-28 or IL-29 is determined. A 50% reduction in viral load in any organ, especially liver, would be significant. For IL-28 or IL-29 treated mice, a 20% increase in the percentage of tetramer positive T cells that proliferate, make cytokine, or display a mature phenotype relative to untreated mice would also be considered significant.

IL-28 or IL-29 injection leading to a reduction in viral load is due to more effective control of viral infection especially in the chronic model where untreated the viral titers remain elevated for an extended period of time. A two fold reduction in viral titer relative to untreated mice is considered significant.

### Example 22

### Influenza Model of Acute Viral Infection

### A. Preliminary Experiment to Test Antiviral Activity

To determine the antiviral activity of IL-28 or IL-29 on acute infection by Influenza virus, an in vivo study using influenza infected c57B1/6 mice is performed using the following protocol:

Animals: 6 weeks-old female BALB/c mice (Charles River) with 148 mice, 30 per group.

Groups:

Absolute control (not infected) to run in parallel for antibody titre and histopathology (2 animals per group)

Vehicle (i.p.) saline

Amantadine (positive control) 10 mg/day during 5 days (per os) starting 2 hours before infection

IL-28 or IL-29 treated (5 μg, i.p. starting 2 hours after infection)

IL-28 or IL-29 (25 μg, i.p. starting 2 hours after infection)

IL-28 or IL-29 (125 μg, i.p. starting 2 hours after infection)

Day 0—Except for the absolute controls, all animals infected with Influenza virus

For viral load (10 at LD50)

For immunology workout (LD30)

Day 0-9—daily injections of IL-28 or IL-29 (i.p.)

Body weight and general appearance recorded (3 times/week)

Day 3—sacrifice of 8 animals per group

Viral load in right lung (TCID50)

Histopathology in left lung

Blood sample for antibody titration

Day 10—sacrifice of all surviving animals collecting blood samples for antibody titration, isolating lung lymphocytes (4 pools of 3) for direct CTL assay (in all 5 groups), and quantitative immunophenotyping for the following markers: CD3/CD4, CD3/CD8, CD3/CD8/CD11b, CD8/CD44/CD62L, CD3/DX5, GR-1/F480, and CD19.

Study No. 2

Efficacy study of IL-28 or IL-29 in C57Bl/6 mice infected with mouse-adapted virus is done using 8 weeks-old female C57Bl/6 mice (Charles River).

Group 1: Vehicle (i.p.)

Group 2: Positive control: Anti-influenza neutralizing antibody (goat anti-influenza A/USSR(H1N1) (Chemicon International, Temecula, Calif.); 40 μg/mouse at 2 h and 4 h post infection (10 μl intranasal)

Group 3: IL-28 or IL-29 (5 μg, i.p.)

Group 4: IL-28 or IL-29 (25 μg, i.p.)

Group 5: IL-28 or IL-29 (125 μg, i.p.)

Following-life observations and immunological workouts are prepared:

Day 0—all animals infected with Influenza virus (dose determined in experiment 2)

Day 0-9—daily injections of IL-28 or IL-29 (i.p.)

Body weight and general appearance recorded every other day

Day 10—sacrifice of surviving animals and perform viral assay to determine viral load in lung.

Isolation of lung lymphocytes (for direct CTL assay in the lungs using EL-4 as targets and different E:T ratio (based on best results from experiments 1 and 2).

Tetramer staining: The number of CD8+ T cells binding MHC Class I tetramers containing influenza A nucleoprotein (NP) epitope are assessed using complexes of MHC class I with viral peptides: FLU-NP₃₆₆₋₃₇₄/D^{b} (ASNENMETM), (LMCV peptide/D^{b}).

Quantitative immunophenotyping of the following: CD8, tetramer, intracellular IFNγ, NK1.1, CD8, tetramer, CD62L, CD44, CD3(+ or −), NK1.1(+), intracellular IFNγ, CD4, CD8, NK1.1, DX5, CD3 (+ or −), NK1.1, DX5, tetramer, Single colour samples for cytometer adjustment.

Survival/Re-Challenge Study

Day 30: Survival study with mice are treated for 9 days with different doses of IL-28 or IL-29 or with positive anti-influenza antibody control. Body weight and antibody production in individual serum samples (Total, IgG1, IgG2a, IgG2b) are measured.

Re-Challenge Study:

Day 0: Both groups will be infected with A/PR virus (1LD30).

Group 6 will not be treated.

Group 7 will be treated for 9 days with 125 μg of IL-28 or IL-29.

Day 30: Survival study

Body weight and antibody production in individual serum samples (Total, IgG1, IgG2a, IgG2b) are measured.

Day 60: Re-challenge study

Survivors in each group will be divided into 2 subgroups

Group 6A and 7A will be re-challenge with A/PR virus (1 LD30)

Group 6B and 7B will be re-challenge with A/PR virus (1 LD30).

Both groups will be followed up and day of sacrifice will be determined. Body weight and antibody production in individual serum samples (Total, IgG1, IgG2a, IgG2b) are measured.

### Example 23

### IL-28 and IL-29 have Antiviral Activity Against Hepatitis B Virus (HBV) In Vivo

A transgenic mouse model (Guidotti et al., J. Virology 69:6158-6169, 1995) supports the replication of high levels of infectious HBV and has been used as a chemotherapeutic model for HBV infection. Transgenic mice are treated with antiviral drugs and the levels of HBV DNA and RNA are measured in the transgenic mouse liver and serum following treatment. HBV protein levels can also be measured in the transgenic mouse serum following treatment. This model has been used to evaluate the effectiveness of lamivudine and IFN-α in reducing HBV viral titers.

HBV TG mice (male) are given intraperitoneal injections of 2.5, 25 or 250 micrograms IL-28 or IL-29 every other day for 14 days (total of 8 doses). Mice are bled for serum collection on day of treatment (day 0) and day 7. One hour following the final dose of IL-29 mice undergo a terminal bleed and are sacrificed. Serum and liver are analyzed for liver HBV DNA, liver HBV RNA, serum HBV DNA, liver HBc, serum Hbe and serum HBs.

Reduction in liver HBV DNA, liver HBV RNA, serum HBV DNA, liver HBc, serum Hbe or serum HBs in response to IL-28 or IL-29 reflects antiviral activity of these compounds against HBV.

### Example 24

### IL-28 and IL-29 Inhibit Human Herpesvirus-8 (HHV-8) Replication in BCBL-1 Cells

The antiviral activities of IL-28 and IL-29 were tested against HHV-8 in an in vitro infection system using a B-lymphoid cell line, BCBL-1.

In the HHV-8 assay the test compound and a ganciclovir control were assayed at five concentrations each, diluted in a half-log series. The endpoints were TaqMan PCR for extracellular HHV-8 DNA (IC50) and cell numbers using CellTiter96® reagent (TC50; Promega, Madison, Wis.). Briefly, BCBL-1 cells were plated in 96-well microtiter plates. After 16-24 hours the cells were washed and the medium was replaced with complete medium containing various concentrations of the test compound in triplicate. Ganciclovir was the positive control, while media alone was a negative control (virus control, VC). Three days later the culture medium was replaced with fresh medium containing the appropriately diluted test compound. Six days following the initial administration of the test compound, the cell culture supernatant was collected, treated with pronase and DNAse and then used in a real-time quantitative TaqMan PCR assay. The PCR-amplified HHV-8 DNA was detected in real-time by monitoring increases in fluorescence signals that result from the exonucleolytic degradation of a quenched fluorescent probe molecule that hybridizes to the amplified HHV-8 DNA. For each PCR amplification, a standard curve was simultaneously generated using dilutions of purified HHV-8 DNA. Antiviral activity was calculated from the reduction in HHV-8 DNA levels (IC₅₀). A novel dye uptake assay was then employed to measure cell viability which was used to calculate toxicity (TC₅₀). The therapeutic index (TI) is calculated as TC₅₀/IC₅₀.

IL-28 and IL-29 inhibit HHV-8 viral replication in BCBL-1 cells. IL-28A had an IC₅₀ of 1 μg/ml and a TC₅₀ of >10 μg/ml (TI>10). IL-29 had an IC₅₀ of 6.5 μg/ml and a TC₅₀ of >10 μg/ml (TI>1.85). MetIL-29C172S-PEG had an IC₅₀ of 0.14 μg/ml and a TC₅₀ of >10 μg/ml (TI >100).

### Example 25

### IL-28 and IL-29 Antiviral Activity Against Epstein Barr Virus (EBV)

The antiviral activities of IL-28 and IL-29 are tested against EBV in an in vitro infection system in a B-lymphoid cell line, P3HR-1. In the EBV assay the test compound and a control are assayed at five concentrations each, diluted in a half-log series. The endpoints are TaqMan PCR for extracellular EBV DNA (IC50) and cell numbers using CellTiter96® reagent (TC50; Promega). Briefly, P3HR-1 cells are plated in 96-well microtiter plates. After 16-24 hours the cells are washed and the medium is replaced with complete medium containing various concentrations of the test compound in triplicate. In addition to a positive control, media alone is added to cells as a negative control (virus control, VC). Three days later the culture medium is replaced with fresh medium containing the appropriately diluted test compound. Six days following the initial administration of the test compound, the cell culture supernatant is collected, treated with pronase and DNAse and then used in a real-time quantitative TaqMan PCR assay. The PCR-amplified EBV DNA is detected in real-time by monitoring increases in fluorescence signals that result from the exonucleolytic degradation of a quenched fluorescent probe molecule that hybridizes to the amplified EBV DNA. For each PCR amplification, a standard curve was simultaneously generated using dilutions of purified EBV DNA. Antiviral activity is calculated from the reduction in EBV DNA levels (IC₅₀). A novel dye uptake assay was then employed to measure cell viability which was used to calculate toxicity (TC₅₀). The therapeutic index (TI) is calculated as TC₅₀/IC₅₀.

### Example 26

### IL-28 and IL-29 Antiviral Activity Against Herpes Simplex Virus-2 (HSV-2)

The antiviral activities of IL-28 and IL-29 were tested against HSV-2 in an in vitro infection system in Vero cells. The antiviral effects of IL-28 and IL-29 were assessed in inhibition of cytopathic effect assays (CPE). The assay involves the killing of Vero cells by the cytopathic HSV-2 virus and the inhibition of cell killing by IL-28 and IL-29. The Vero cells are propagated in Dulbecco's modified essential medium (DMEM) containing phenol red with 10% horse serum, 1% glutamine and 1% penicillin-streptomycin, while the CPE inhibition assays are performed in DMEM without phenol red with 2% FBS, 1% glutamine and 1% Pen-Strep. On the day preceding the assays, cells were trypsinized (1% trypsin-EDTA), washed, counted and plated out at 10⁴ cells/well in a 96-well flat-bottom BioCoat® plates (Fisher Scientific, Pittsburgh, Pa.) in a volume of 100 μl/well. The next morning, the medium was removed and a pre-titered aliquot of virus was added to the cells. The amount of virus used is the maximum dilution that would yield complete cell killing (>80%) at the time of maximal CPE development. Cell viability is determined using a CellTiter 96® reagent (Promega) according to the manufacturer's protocol, using a Vmax plate reader (Molecular Devices, Sunnyvale, Calif.). Compounds are tested at six concentrations each, diluted in assay medium in a half-log series. Acyclovir was used as a positive control. Compounds are added at the time of viral infection. The average background and drug color-corrected data for percent CPE reduction and percent cell viability at each concentration are determined relative to controls and the IC₅₀ calculated relative to the TC₅₀.

IL-28A, IL-29 and MetIL-29C172S-PEG did not inhibit cell death (IC₅₀ of >10 ug/ml) in this assay. There was also no antiviral activity of IFNα in the assay.

### Example 27

### PEG-rIL-29-C172S and PEG-rIL-29-d2-7 Antiviral Activity Against Hepatitis C Virus in the Hepatitis C Replicon Model

In order to determine the effectiveness of PEG-rIL-29 in preventing viral replication of human hepatitis C virus two forms of PEG-rIL-29 (IL-29 C172S polypeptide N-terminally conjugated to a 20 kD methoxy-polyethylene glycol propionaldehyde (“PEG-rIL-29-C1725”) (SEQ ID NO:34) and IL-29 C172S d2-7 polypeptide N-terminally conjugated to a 20 kD methoxy-polyethylene glycol propionaldehyde (“PEG-rIL-29-d2-7”) (SEQ ID NO:134) were tested in the HCV Replicon model.

In this model AVA5 cells (Huh7 cells containing the subgenomic HCV replicon, BB7) (Blight et al., Science, 290:1972-1974 (2000)) were used. Cultures were maintained in a sub-confluent state in DMEM with glutamine, non-essential amino acids, and 10% heat-inactivated fetal bovine serum (Biofluids, Inc.) as previously described (Blight et al., Science, 290:1972-1974 (2000)). Stock cultures were maintained in a sub-confluent state in this culture medium with 1 mg/ml G418 (Invitrogen, Inc.) (Blight et al., Science, 290:1972-1974 (2000)). Cells for antiviral analysis were seeded into 24-well or 48-well tissue culture plates (Nunc, Inc.) and grown for three days in the presence of G418. G418 was then removed for the duration of the antiviral treatments to eliminate potential loss of cells due to the reduction of HCV replicon (and G418-resistance) copy number. Cultures (rapidly dividing, 3-4 cultures per concentration, per experiment) were treated for three consecutive days with the test compounds. Medium was replaced daily with fresh test compounds. Analysis of HCV RNA was performed 24 hours following the last addition of test compounds. Toxicity analyses using neutral red dye uptake were performed as previously described in Korba et al., Antiviral Res., 19(1):55-70 (1992). Cultures for the toxicity analyses were seeded from the same stock cultures and maintained on separate plates under conditions identical to those used for the corresponding antiviral assays.

Daily aliquots of test compounds (PEG-rIL-29-C172S and PEG-rIL-29-d2-7, PEGASYS® (Roche) and PEG®-Intron (Schering-Plough)) were made from stock solutions in individual tubes. On each day of treatment, daily aliquots of the diluted test compounds were suspended into culture medium at room temperature, and immediately added to the cell cultures, thereby subjecting each aliquot of test compound to the same, limited, number of freeze-thaw cycles.

HCV RNA levels were quantitatively measured using one of two methods. The first method used the application of commercial bDNA technology (Versant HCV™, Bayer Diagnostics, Inc., Oakland Calif.) for the detection of intracellular HCV. For this assay, no RNA extraction is required. Cells are lysed in the culture wells, and the resulting solution is then directly quantitatively assayed for RNA. The bDNA assay uses the certified HCV international reference standards and has internal extraction controls included in each sample. The EC50 value for each test drug is calculated using linear regression analysis (MS Excel™).

The second method for HCV RNA quantitation is a modification of a previously described dot blot hybridization assay (Korba et al., Antiviral Res., 19(1):55-70 (1992)). Whole cell RNA was extracted from cells using either RNeasy™ mini-columns (Qiagen, Inc.), or Purescript RNA Purification kits (Gentra Systems, Inc.). RNA samples were denatured in 10×SSC/18% deionized formaldehyde for 20 min. at 80° C., applied to nitrocellulose under vacuum, washed once with 20×SSC, baked for 15 min at 80° C. under vacuum, and hybridized against ³²P-labelled DNA probes. Following the denaturation step, each RNA sample was split onto two nitrocellulose membranes for hybridization with either HCV-specific or human β-actin-specific ³²P-labelled DNA probes (95% of the sample for HCV, 5% for β-actin). The HCV hybridization probe used was a gel-purified, 6600 bp Hind III fragment isolated from the HCV replicon source plasmid, BB7 (Blight et al., Science, 290:1972-1974 (2000)). The β-actin probe was a gel-purified, 550 bp PCR product generated from AVA5 cell RNA using a commercial PCR kit (Invitrogen, Inc.). Both probes were labeled with ³²P-dCTP using a commercial random priming procedure (Clonetech-BD Biosciences, Inc.). Hybridization was performed overnight at either 47° C. (HCV), or 40° C. (β-actin), and washing was performed at either 65° C. (HCV), or 60° C. (β-actin), as previously described (Korba et al., Antiviral Res., 19(1):55-70 (1992)). Quantitation against independently determined standards present on each hybridization membrane was achieved using a beta scanner (Packard Instruments, Inc.). The mean levels of β-actin RNA present in 6-8 untreated cultures contained in each experiment were used as the basis for determining the relative level of β-actin RNA in each individual sample. Levels of HCV RNA were normalized to the levels of β-actin RNA present in each individual sample. HCV RNA levels in treated cultures were then compared to the normalized mean levels of HCV RNA present in the 6-8 untreated cultures contained in each experiment. The EC50 value for each test drug is calculated using linear regression analysis (MS Excel™).

Initial experiments tested PEG-rIL-29-C1725 (SEQ ID NO:34), PEGASYS® and PEG®-Intron and used the Versant HCV™ method to measure HCV viral load. In these experiments the calculated EC50s were PEG-rIL-29-C1725, 0.117 ng/mL; PEGASYS®, 0.004 ng/mL; PEG®-Intron, 0.002 ng/mL. All cytokines tested reduced HCV viral load by greater than 99% at the maximum concentration tested (PEG-rIL-29-C172S, 99.9% HCV RNA reduction at 1000 ng/mL; PEGASYS®, 99.78% HCV RNA reduction at 1000 ng/mL; PEG®-Intron, 99.85% HCV RNA reduction at 1000 ng/mL).

Additional experiments tested PEG-rIL-29-C172S, PEG-rIL-29-d2-7, PEGASYS® and PEG®-Intron and used the dot blot hybridization assay described above. In these experiments the calculated EC50s were PEG-rIL-29-C1725, 0.356 ng/mL; PEG-rIL-29-d2-7, 0.516 ng/mL; PEGASYS®, 0.004 ng/mL; PEG®-Intron, 0.002 ng/mL. All cytokines tested reduced HCV viral load by greater than 90% at 100 ng/mL, the maximum concentration tested.

In conclusion, PEG-rIL-29-C172S and PEG-rIL-29-d2-7 are able to reduce HCV viral load in a dose-dependent manner in the HCV Replicon model similar to that of the marketed pegylated interferon alphas, PEGASYS® and PEG®-Intron.

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating a hepatitis C infection in a mammal, the method comprising:
administering to the mammal a therapeutically effective amount of an isolated polypeptide comprising amino acid residues 1-176 of SEQ ID NO:138, which is encoded by the polynucleotide of SEQ ID NO:137, wherein after administration of the encoded polypeptide the hepatitis C load is reduced.

2. The method of claim 1 wherein the polypeptide is conjugated to a polyalkyl oxide moiety.

3. The method of claim 2 wherein the polyalkyl oxide moiety is polyethylene glycol.

4. The method of claim 3 wherein the polyethylene glycol is monomethoxy-PEG propionaldehyde.

5. The method of claim 4 wherein the monomethoxy-PEG propionaldehyde has a molecular weight of about 20 Kd or 30 Kd.

6. The method of claim 4 wherein the monomethoxy-PEG propionaldehyde is linear or branched.

7. The method of claim 4 wherein the monomethoxy-PEG propionaldehyde is conjugated N-terminally to the polypeptide.

8. A method of treating a hepatitis C infection in a mammal, the method comprising:
administering to the mammal a therapeutically effective amount of a composition comprising an isolated polypeptide comprising amino acid residues 1-176 of SEQ ID NO:138, which is encoded by the polynucleotide of SEQ ID NO:137, wherein after administration of the composition the hepatitis C load is reduced.

9. The method of claim 8 wherein the polypeptide is conjugated to a polyalkyl oxide moiety.

10. The method of claim 9 wherein the polyalkyl oxide moiety is polyethylene glycol.

11. The method of claim 10 wherein the polyethylene glycol is monomethoxy-PEG propionaldehyde.

12. The method of claim 11 wherein the monomethoxy-PEG propionaldehyde has a molecular weight of about 20 Kd or 30 Kd.

13. The method of claim 11 wherein the monomethoxy-PEG propionaldehyde is linear or branched.

14. The method of claim 11 wherein the monomethoxy-PEG propionaldehyde is conjugated N-terminally to the polypeptide.
